(19) United States
(12) Patent Application Publication
(10) Pub. No.: US 2026/0146155 A1
(43) Pub. Date: May 28, 2026

(54) GELLING SOLUTIONS FOR ADMINISTRATION OF COMPOUNDS TO THE INNER EAR

(71) Applicant: Spiral Therapeutics Inc., South San Francisco, CA (US)

(72) Inventors: Eugene de Juan, JR., South San Francisco, CA (US); Signe Erickson, South San Francisco, CA (US); Andrew Ayoob, San Francisco, CA (US); Kathleen Cogan Farinas, Los Altos, CA (US)

(21) Appl. No.: 19/459,988

(22) Filed: Jan. 26, 2026

Related U.S. Application Data
(62) Division of application No. 17/923,945.
(60) Provisional application No. 63/024,232, filed on May 13, 2020.

(51) Int. Cl. C08L 71/00; A61K 9/00; A61K 9/06; A61K 31/573; A61P 27/16; C08L 77/04

(52) U.S. Cl. CPC C08L 71/00; A61K 9/0046; A61K 9/06; A61K 31/573; A61P 27/16; C08L 77/04

(57) ABSTRACT

Provided herein are polymer compositions and extended release otic agents. In one aspect, provided herein is a polymer composition including about 5% to about 15% by weight of the polymer composition of a functional polymer, wherein the functional polymer includes a first functional group, about 0.05% to about 0.6% by weight of the polymer composition of a crosslinker, wherein the crosslinker includes a second functional group, and water, wherein a crosslinking reaction can occur between the first functional group and the second functional group to form a gel, and wherein the polymer composition has a gelation time of about 45 seconds to about 60 minutes at a temperature of about 20° C.

### CROSS-REFERENCE TO RELATED APPLICATIONS

This application is a division of U.S. patent application Ser. No. 17/923,945 filed on Nov. 8, 2022, which is a National Stage Application under 35 U.S.C. § 371 and claims the benefit of International Application No. PCT/US2021/031977 filed on May 12, 2021, which claims priority to U.S. Provisional Application Ser. No. 63/024,232 filed on May 13, 2020, and each of these related applications is incorporated herein by reference in its entirety.

### DESCRIPTION OF SEQUENCE LISTING SUBMITTED ELECTRONICALLY

This application contains a Sequence Listing that has been submitted electronically as an XML file named 50051-0011002_ST_ST26.xml. The XML file was created on Jan. 26, 2026 based upon the material from the original TXT file previously submitted in the aforementioned related applications (50051_0011WO1.txt, recorded on May 12, 2021), and the XML file is 13,341 bytes in size. The material in the XML file is hereby incorporated by reference in its entirety.

### FIELD OF THE INVENTION

The disclosure relates to formulations for treatment of inner ear conditions or disease, particularly solutions which form a stable hydrogel at body temperature to provide controlled delivery over a period of time of therapeutic, prophylactic and/or diagnostic agents.

### BACKGROUND OF THE INVENTION

The inner ear can be difficult to treat effectively. For example, the inner ear accounts for only 0.004% of the average circulating blood volume and is encapsulated in one of the densest bones in the body. These, combined with the presence of the blood-labyrinth barrier (BLB), limit access of most therapeutic compounds to the inner ear. Oral, intravenous, and intramuscular routes of administration can be inefficient and can require high doses, risking systemic side effects.

### SUMMARY OF THE INVENTION

This document is based, at least in part, on compositions that can be used to deliver an active agent to the middle and/or inner ear of a subject.

Provided herein can be a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein a crosslinking reaction can occur between the first functional group and the second functional group to form a gel, and wherein the polymer composition can have a gelation time of about 45 seconds to about 60 minutes at a temperature of about 20° C.

Also provided herein is a polymer composition including about 5% to about 15% by weight of the polymer composition of a functional polymer, wherein the functional polymer includes a first functional group, about 0.2% to about 0.6% by weight of the polymer composition of a crosslinker, wherein crosslinker includes a second functional group, and water, wherein a crosslinking reaction can occur between the first functional group and the second functional group to form a gel, and wherein the gel, when formed in the middle ear, can have a residence time of at least 5 days.

Also provided herein is a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein a crosslinking reaction can occur between the first functional group and the second functional group to form a gel, and wherein gel has a gel duration of at least 5 days at 37° C.

Additionally provided herein is a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein the polymer composition can have a pH of about 5.5 to about 8.5, and wherein a crosslinking reaction can occur between the first functional group and the second functional group to form a gel.

Provided herein is also a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein a crosslinking reaction can occur between the first functional group and the second functional group to form a gel, and wherein the gel, following equilibration in phosphate-buffered saline (PBS) for 2 days, swells less than 100%.

Also provided herein a polymer composition including about 5% to about 15% by weight of the polymer composition amount of a functional polymer, wherein the functional polymer includes a first functional group, about 0.2% to about 0.6% by weight of the polymer composition of a crosslinker, wherein crosslinker includes a second functional group, and water, wherein a crosslinking reaction can occur between the first functional group and the second functional group to form a gel, and wherein the gel is elastic.

Also provided herein is a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein a crosslinking reaction can occur between the first functional group and the second functional group to form a gel, and wherein gel can be mucoadhesive.

Additionally provided herein is a polymer composition including about 5% to about 15% by weight of the polymer composition of a functional polymer, wherein the functional polymer includes a first functional group, about 0.2% to about 0.6% by weight of the polymer composition of a crosslinker, wherein the crosslinker includes a second functional group, and water, wherein the polymer composition can have a viscosity of about 1 mPa·s to about 1000 mPa·s, and wherein a crosslinking reaction can occur between the first functional group and the second functional group to form a gel.

In some embodiments of any of the polymer compositions herein, the polymer composition can have a gelation time of about 45 seconds to about 60 minutes at a temperature of about 20° C. In some embodiments of any of the polymer compositions herein, the gel, when formed in the middle ear, can have a residence time of at least 5 days. In some embodiments of any of the polymer compositions herein, the gel can have a gel duration of at least 5 days at 37° C. In some embodiments of any of the polymer compositions herein, the polymer composition can have a pH of about 5.5 to about 8.5. In some embodiments of any of the polymer compositions herein, the gel, following equilibration in phosphate-buffered saline (PBS) for 2 days, swells less than 100%. In some embodiments of any of the polymer compositions herein, the gel can be elastic. In some embodiments of any of the polymer compositions herein, the gel can be mucoadhesive. In some embodiments of any of the polymer compositions herein, the polymer composition can have a viscosity of about 1 mPa·s to about 1000 mPa·s.

In some embodiments of any of the polymer compositions herein, the polymer composition can include about 8% to about 12% by weight of the polymer composition of the functional polymer. In some embodiments of any of the polymer compositions herein, the polymer composition can include about 10% by weight of the polymer composition of the functional polymer. In some embodiments of any of the polymer compositions herein, polymer composition can include about 0.3% to about 0.5% by weight of the polymer composition of the crosslinker. In some embodiments of any of the polymer compositions herein, the polymer composition can include about 0.4% to about 0.6% by weight of the polymer composition of the crosslinker.

In some embodiments of any of the polymer compositions herein, the polymer composition can have a gelation time of about 5 minutes to about 20 minutes at a temperature of about 20° C. In some embodiments of any of the polymer compositions herein, the polymer composition can have a gelation time of about 8 minutes to about 12 minutes at a temperature of about 20° C. In some embodiments of any of the polymer compositions herein, the polymer composition can have a gelation time of about 10 seconds to about 30 minutes at a temperature of about 37° C. In some embodiments of any of the polymer compositions herein, the polymer composition can have a gelation time of about 2 minutes to about 8 minutes at a temperature of about 37° C. In some embodiments of any of the polymer compositions herein, the gel, when formed in the middle ear, can have a residence time of at least 1 week. In some embodiments of any of the polymer compositions herein, the gel, when formed in the middle ear, can have a residence time of at least 2 weeks. In some embodiments of any of the polymer compositions herein, the gel, when formed in the middle ear, can have a residence time of at least 1 month. In some embodiments of any of the polymer compositions herein, the gel, when formed in the middle ear, can have a residence time of at least 2 months. In some embodiments of any of the polymer compositions herein, the polymer composition can have a pH of about 6.4 to about 7.4. In some embodiments of any of the polymer compositions herein, the polymer composition can have a pH of about 6.0 and 7.0. In some embodiments of any of the polymer compositions herein, the gel, following equilibration in phosphate-buffered saline (PBS) for 2 days, swells less than 80%. In some embodiments of any of the polymer compositions herein, the gel, following equilibration in phosphate-buffered saline (PBS) for 2 days, swells less than 60%. In some embodiments of any of the polymer compositions herein, the polymer composition can have a viscosity of about 1 mPa·s to about 100 mPa·s. In some embodiments of any of the polymer compositions herein, the polymer composition can have a viscosity of about 1 mPa·s to about 50 mPa·s. In some embodiments of any of the polymer compositions herein, the gel can be hypotonic to the endolymph or perilymph. In some embodiments of any of the polymer compositions herein, the gel can be isotonic to the endolymph or perilymph. In some embodiments of any of the polymer compositions herein, the gel can be hypertonic to the endolymph or perilymph. In some embodiments of any of the polymer compositions herein, the gel can have a pH of about 6.0 to about 7.7. In some embodiments of any of the polymer compositions herein, the gel can have a pH of about 6.6 to about 6.8.

In some embodiments of any of the polymer compositions herein, the ratio of the first functional group to the second functional group can be about 0.9:1 to about 1:0.9. In some embodiments of any of the polymer compositions herein, the ratio of the first functional group to the second functional group can be about 1:1. In some embodiments of any of the polymer compositions herein, the functional polymer can be a modified PEG.

In some embodiments of any of the polymer compositions herein, the first functional group includes an electrophile and the second functional group includes a nucleophile. In some embodiments of any of the polymer compositions herein, the first functional group includes a succinimidyl ester. In some embodiments of any of the polymer compositions herein, the second functional group includes a primary amine. In some embodiments of any of the polymer compositions herein, the functional polymer can be pentaerythritol poly(ethylene glycol) ether tetrasuccinimidyl glutarate. In some embodiments of any of the polymer compositions herein, the crosslinker includes polylysine. In some embodiments of any of the polymer compositions herein, the crosslinker includes trilysine.

In some embodiments of any of the polymer compositions herein, first functional group includes a nucleophile and the second functional group includes an electrophile. In some embodiments of any of the polymer compositions herein, the first functional group includes a primary amine. In some embodiments of any of the polymer compositions herein, the second functional group includes a succinimidyl ester.

Also provided herein is an extended release otic composition including any one or more of the polymer compositions described herein, and an active agent.

In some embodiments, the active agent can be selected from the group consisting of a therapeutic agent, a prophylactic agent, a diagnostic or visualization agent, and combinations thereof. In some embodiments, the therapeutic agent or prophylactic agent can be selected from the group consisting of a protein, a carbohydrate, a nucleic acid, a small molecule, and combinations thereof. In some embodiments, the protein can be selected from the group consisting of an enzyme, a growth factor, an antibody or an antigen-binding fragment thereof, and combinations thereof. In some embodiments, the carbohydrate can be a glycosaminoglycan. In some embodiments, the nucleic acid can be selected from the group consisting of an antisense oligonucleotide, an aptamer, a micro RNA, a short interfering RNA, a ribozyme, and combinations thereof. In some embodiments, the small molecule can be selected from the group consisting of an antibiotic, an antineoplastic agent, a local anesthetic, a steroid, a hormone, an anti-apoptotic agent, an angiogenic agent, an anti-angiogenic agent, a neurotransmitter, a psychoactive drug, an anti-inflammatory, and combinations thereof. In some embodiments, the small molecule can be an inhibitor of Apaf-1.

In some embodiments, the active agent can be a tyrosine kinase inhibitor. In some embodiments, the active agent can be a VEGF inhibitor. In some embodiments, the VEGF inhibitor can be selected from the group consisting of agerafenib, altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof. In some embodiments, the VEGF inhibitor includes an antibody or antigen-binding fragment thereof. In some embodiments, the antibody or antigen-binding fragment thereof can be selected from the group consisting of alacizumab, bevacizumab, icrucumab, ramucirumab, ranibizumab, and combinations thereof. In some embodiments, the VEGF inhibitor includes a decoy receptor. In some embodiments, the decoy receptor can be aflibercept. In some embodiments, the VEGF inhibitor includes an allosteric modulator of a VEGFR. In some embodiments, the allosteric modulator can be cyclotraxin B. In some embodiments, the VEGF inhibitor can be at least 10-fold selective for VEGFR2 over another VEGFR. In some embodiments, the VEGF inhibitor can be at least 20-fold selective for VEGFR2 over another VEGFR. In some embodiments, the VEGF inhibitor can be at least 50-fold selective for VEGFR2 over another VEGFR.

In some embodiments, the tyrosine kinase inhibitor or VEGF inhibitor can be present in an amount sufficient to reduce edema and lymphatic dysfunction in an affected ear. In some embodiments, the active agent includes an anti-inflammatory. In some embodiments, the active agent includes a steroid. In some embodiments, the active agent does not comprise a steroid.

In some embodiments, the active agent includes a diagnostic or visualization agent. In some embodiments, the diagnostic or visualization agent can be selected from the group consisting of a dye, a fluorophore, an MRI contrast agent, and combinations thereof.

In some embodiments, the active agent can be present in the extended release otic composition in the form of microparticles. In some embodiments, the active agent can be present in the extended release otic composition in the form of nanoparticles.

In some embodiments, the active agent can be present in an amount of about 0.01% to about 40% by weight of the polymer composition. In some embodiments, the active agent can be present in an amount of about 0.1% to about 20% by weight of the polymer composition. In some embodiments, the active agent can be present in an amount of about 1% to about 10% by weight of the polymer composition.

In some embodiments, an extended release otic composition can further include an excipient. In some embodiments, the excipient can be selected from the group consisting of a buffer, a tonicity agent, a mucoadhesive agent, a stabilizing agent, a preservative, a carriers, a penetration enhancer, a diluent, a dispersing agent, a viscosity modifying agent, a solubilizer, an osmolarity modifying agent, and combinations thereof.

Also provided herein is a gel formed by any one or more of the polymer compositions described herein. Also provided herein is a gel formed by any one or more of the extended release otic compositions described herein.

Also provided herein is manufacture of a medicament including any one or more of the extended release otic compositions described herein for the treatment of an otic disease or disorder.

Provided herein is a method of preparing an extended release otic composition, the method including combining a solution or suspension of a functional polymer, wherein the functional polymer comprises a first functional group, a solution or suspension of a crosslinker, wherein the crosslinker comprises a second functional group, and an active agent to form an extended release otic composition, such that the functional polymer can be present in an amount of about 5% to about 15% by weight of the extended release otic composition and the crosslinker can be present in the extended release otic composition in an amount of about 0.2% to about 0.6% by weight of the extended release otic composition, wherein a crosslinking reaction can occur between the first functional group and the second functional group to form a gel.

Provided herein is a method of preparing an extended release otic composition, the method including (a) making a solution or suspension of a functional polymer, wherein the functional polymer comprises a first functional group, (b) making a solution or suspension of a crosslinker, wherein the crosslinker comprises a second functional group, and (c) combining the solution or suspension of the functional polymer and the solution or suspension of the crosslinker to form an extended release otic composition, such that the functional polymer can be present in an amount of about 5% to about 15% by weight of the extended release otic composition and the crosslinker can be present in the extended release otic composition in an amount of about 0.2% to about 0.6% by weight of the extended release otic composition, wherein a crosslinking reaction can occur between the first functional group and the second functional group to form a gel.

Provided herein is a method preparing an extended release otic composition, the method including (a) making a solution or suspension of a functional polymer, wherein the functional polymer comprises a first functional group, and (b) combining the solution or suspension of the functional polymer with a solution or suspension of a crosslinker, wherein the crosslinker comprises a second functional group, such that the functional polymer can be present in an amount of about 5% to about 15% by weight of the extended release otic composition and the crosslinker can be present in the extended release otic composition in an amount of about 0.2% to about 0.6% by weight of the extended release otic composition, wherein a crosslinking reaction can occur between the first functional group and the second functional group to form a gel.

Also provided herein is a method of preparing an extended release otic composition, the method including (a) making a solution or suspension of a functional polymer, wherein the functional polymer comprises a first functional group, (b) altering the pH of a solution or suspension of a crosslinker, wherein the crosslinker comprises a second functional group, and (c) combining the solution or suspension of the functional polymer with the solution or suspension of the crosslinker, wherein a crosslinking reaction can occur between the first functional group and the second functional group to form a gel.

Additionally provided herein is a method of preparing an extended release otic composition, the method including (a) making a solution or suspension of a crosslinker, wherein the crosslinker comprises a second functional group, and (b) combining the solution or suspension of the crosslinker with a solution or suspension of a functional polymer, wherein the functional polymer comprises a first functional group, such that the functional polymer can be present in an amount of about 5% to about 15% by weight of the extended release otic composition and the crosslinker can be present in the extended release otic composition in an amount of about 0.2% to about 0.6% by weight of the extended release otic composition, wherein a crosslinking reaction can occur between the first functional group and the second functional group to form a gel.

Provided herein is a method of preparing an extended release otic composition, the method including (a) making a solution or suspension of a crosslinker, wherein the crosslinker comprises a second functional group, (b) altering the pH of a solution or suspension of a functional polymer, wherein the functional polymer comprises a first functional group, and (c) combining the solution or suspension of the functional polymer with the solution or suspension of the crosslinker, wherein a crosslinking reaction can occur between the first functional group and the second functional group to form a gel.

In some embodiments of any of the methods of preparing provided herein, the active agent can be present in the solution or suspension of the functional polymer. In some embodiments of any of the methods of preparing provided herein, the active agent can be combined with the functional polymer prior to making the solution or suspension of the functional polymer. In some embodiments of any of the methods of preparing provided herein, the active agent can be combined with the solution or suspension of the functional polymer. In some embodiments of any of the methods of preparing provided herein, combining the solution or suspension of the functional polymer with the solution or suspension of the crosslinker includes combining the solution or suspension of the functional polymer, the solution or suspension of the crosslinker, and an active agent. In some embodiments of any of the methods of preparing provided herein, the active agent can be provided as a solid. In some embodiments of any of the methods of preparing provided herein, the active agent can be provided as a solution or suspension. In some embodiments of any of the methods of preparing provided herein, the extended release otic composition can be any one or more of the extended release otic compositions provided herein.

Provided herein is a method of treating an otic disease or disorder in a subject, the method including identifying a subject as having an otic disease or disorder, and administering a therapeutically effective amount of any one or more of the extended release otic compositions provided herein an affected ear of the subject.

Also provided herein is a method of treating an otic disease or disorder in a subject, the method including administering a therapeutically effective amount of any one or more of the extended release otic compositions provided herein to an ear of a subject in need thereof.

Also provided herein is a method of treating an otic disease or disorder in a subject, the method including (i) preparing an extended release otic composition by any one or more of the methods provided herein, and (ii) administering a therapeutically effective amount of the extended release otic composition to an ear of a subject in need thereof.

Additionally provided herein is a method of treating an otic disease or disorder in a subject, the method including (i) identifying a subject as having an otic disease or disorder, (ii) preparing an extended release otic composition by any one or more of the methods described herein, and (iii) administering a therapeutically effective amount of the extended release otic composition to an affected ear of the subject.

In some embodiments of any of the methods of treating provided herein, the otic disease or disorder can be otic disease or disorder can be selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof. In some embodiments of any of the methods of treating provided herein, the sensorineural hearing loss can be sudden sensorineural hearing loss. In some embodiments of any of the methods of treating provided herein, the sensorineural hearing loss can be associated with diabetes.

Also provided herein is a method of treating Ménière's Disease in a subject, the method including administering a therapeutically effective amount of any one or more of the extended release otic compositions described herein to an ear of a subject in need thereof.

Provided herein is a method of treating Ménière's Disease in a subject, the method including (i) identifying a subject as having Ménière's Disease, and (ii) administering a therapeutically effective amount of any one or more of the extended release otic compositions described herein to an affected ear of the subject.

Additionally provided herein is a method of treating Ménière's Disease in a subject, the method including (i) preparing an extended release otic composition by any one or more of the methods described herein, and (ii) administering a therapeutically effective amount of the extended release otic composition to an ear of a subject in need thereof.

Also provided herein is a method of treating Ménière's Disease in a subject, the method including (i) identifying a subject as having Ménière's Disease, (ii) preparing an extended release otic composition by any one or more of the methods described herein, and (iii) administering a therapeutically effective amount of the extended release otic composition to an affected ear of the subject.

In any one or more of the methods of treating provided herein, the administering occurs less than 10 minutes after combining the solution or suspension of the functional polymer with the solution or suspension of the crosslinker. In any one or more of the methods of treating provided herein, the administering occurs less than 5 minutes after combining the solution or suspension of the functional polymer with the solution or suspension of the crosslinker. In any one or more of the methods of treating provided herein, the administering can include administering between about 5 μL and about 500 μL of the extended release otic composition. In any one or more of the methods of treating provided herein, the administering can include administering between about 50 μL and about 200 μL of the extended release otic composition. In any one or more of the methods of treating provided herein, the administering can include injecting through the tympanic membrane.

Provided herein is a method of treating an otic disease or disorder in a subject, the method including identifying a subject as having an otic disease or disorder, and administering a therapeutically effective amount of tyrosine kinase inhibitor to the subject.

In some embodiments, the otic disease or disorder can be selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof. In some embodiments, the sensorineural hearing loss can be sudden sensorineural hearing loss. In some embodiments, the sensorineural hearing loss can be associated with diabetes.

Also provided herein is a method of treating Ménière's Disease in a subject, the method including (i) identifying a subject as having Ménière's Disease, and (ii) administering a therapeutically effective amount of tyrosine kinase inhibitor to the subject.

Provided herein is a method of treating Ménière's Disease in a subject, the method including administering a therapeutically effective amount of tyrosine kinase inhibitor to a subject in need thereof.

In some embodiments, the administering can include systemic administration. In some embodiments, the administering can include administering to an affected ear of the subject. In some embodiments, the tyrosine kinase inhibitor can include a VEGF inhibitor. In some embodiments, the VEGF inhibitor can be selected from the group consisting of agerafenib, altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof. In some embodiments, the VEGF inhibitor includes an antibody or antigen-binding fragment thereof. In some embodiments, the antibody or antigen-binding fragment thereof can be selected from the group consisting of alacizumab, bevacizumab, icrucumab, ramucirumab, ranibizumab, and combinations thereof. In some embodiments, the VEGF inhibitor includes a decoy receptor. In some embodiments, decoy receptor can be aflibercept. In some embodiments, the VEGF inhibitor includes an allosteric modulator of a VEGFR. In some embodiments, the allosteric modulator of a VEGFR can be cyclotraxin B. In some embodiments, the VEGF inhibitor can be at least 10-fold selective for VEGFR2 over another VEGFR. In some embodiments, the VEGF inhibitor can be at least 20-fold selective for VEGFR2 over another VEGFR. In some embodiments, the VEGF inhibitor can be at least 50-fold selective for VEGFR2 over another VEGFR. In some embodiments, the tyrosine kinase inhibitor can be present in an amount sufficient to reduce edema and lymphatic dysfunction in an affected ear. In some embodiments, the tyrosine kinase inhibitor can be provided in the form of any one or more of the extended release otic compositions described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims. The word “comprising” in the claims may be replaced by “consisting essentially of” or with “consisting of,” according to standard practice in patent law.

### BRIEF DESCRIPTION OF THE DRAWINGS

FIG. 1A is a structure of pentaerythritol poly(ethylene glycol) ether tetrasuccinimidyl glutarate.

FIG. 1B is a structure of trilysine.

FIG. 2A is a graph of the Swelling profiles (percent) over time (days) for pH adjusted PEG-trilysine polymer samples containing 0% (solid squares), 1% (open squares), 3% (diamonds), and 6% (triangles) dexamethasone (Dex) (n=3).

FIG. 2B is a graph of cumulative Drug release (μg) for pH adjusted PEG-trilysine polymer samples containing 6% (triangle), 3% (diamonds) or 1% (squares) dexamethasone (n=3).

FIG. 3 is a graph of the Swelling profiles (percent swelling) for pH adjusted PEG-trilysine polymer samples, pot life 67 minutes (circles), 26 minutes (squares), or 16 minutes (triangles) containing 6 wt % dexamethasone (n=6) over time (days).

FIG. 4 is a graph of Drug release (μg) over time (days) for pH adjusted PEG-trilysine polymer samples containing 6 wt % dexamethasone (n=6), pot life 67 minutes (circles), 26 minutes (squares), or 16 minutes (triangles) containing 6 wt % dexamethasone (n=6)

FIG. 5A is a graph of Drug release (μg) over time (days) for 6 wt % dexamethasone gels in pH adjusted PEG-trilysine polymer (circles) vs Poloxamer 407 (triangles) (n=5) in a PBS solution.

FIG. 5B is a graph of Permeation (Drug release (μg)) over time (days) of 6 wt % dexamethasone gels in pH adjusted PEG-trilysine polymer (circles) vs Poloxamer 407 (triangle) (n=3) through a membrane.

FIG. 6A is a graph of dexamethasone concentration in plasma versus time in an in vivo study. Open symbols are from subjects administered a PEG-trilysine polymer formulation and filled symbols are from subjects administered a P407 formulation.

FIG. 6B is a graph of dexamethasone concentration in perilymph versus time in an in vivo study. Points are individual subjects, for left ears (L, circles, squares, hexagons) and right ears (R, triangles and diamonds). Open symbols are from subjects administered a PEG-trilysine polymer formulation, and filled symbols are from animals administered a P407 formulation. Arrow symbol represents subject A843, administered a P407 formulation, whose dexamethasone level is above the level of detection (limit 9500 ng/mL).

FIG. 6C is the same data as FIG. 6B, plotted with the right and left ears grouped together.

FIG. 7 is a graph of gel time (min) vs pH of various pH-adjusted PEG-trilysine polymer compositions.

FIG. 8 is a graph of gel time (min) vs years since expiration of the PEG-trilysine kit for otic compositions prepared with 0.23 M 50:50 diluent (circles) and 0.25 M 50:50 diluent (triangles).

FIG. 9 is a graph of cumulative release of dexamethasone (mg) vs time (days) for extended release otic compositions D1 (circles), D2 (triangles), D3 (squares), and D4 (diamonds).

FIG. 10A is a graph of threshold shift from baseline (dB) versus days after treatment in an in vivo study.

FIG. 10B is a graph of threshold shift from baseline (dB) versus ABR stimulus frequency in an in vivo study.

FIG. 10C is a graph of threshold shift from baseline (dB) versus ABR stimulus frequency in an in vivo study at 8 weeks post-treatment.

FIG. 11A is a graph of dexamethasone concentration (ng/mL) versus time in plasma for a population of subjects in an in vivo study.

FIG. 11B is a graph of dexamethasone concentration (ng/mL) versus time in plasma for individual subjects in an in vivo study.

### DETAILED DESCRIPTION OF THE INVENTION

Potential side effects of systemic treatment and complications from a long lasting, higher dose therapy can be avoided through topical application therapy. Inner ear therapeutics (e.g., drugs formulated as biocompatible gels) can be delivered via intra-tympanic injections into the middle ear across the tympanic membrane (TM). Passive diffusion of agents from the middle ear to the inner ear following intra-tympanic injection into the inner ear has variable efficacy due to anatomical variations, such as the presence of pseudomembranes covering the round window membrane, failure of the injected formulation to contact the round window membrane and limited permeability of the round window and oval window membranes. This can lead to poor patient outcomes. Additionally, the risk of surgical complications is high. Further, rapid clearance of agents from the perilymph of the inner ear can result in the need for repeated intra-tympanic injections, which are also undesirable for subjects and are associated with cumulative risk of infection, inflammation, and long term damage to the tympanic membrane, in addition to the risk of lower compliance.

Local delivery of therapeutics into the inner ear usually results in higher concentrations in the inner ear fluids than would be the case with systemic application. Substances applied locally (e.g., at a lower dose than would be used for systemic administration) can be administered where there are major restrictions or even contraindications associated with systemic application; see, e.g. Salt, et al. Drug Discov Today. 2005 Oct. 1; 10(19): 1299-1306. Substances are applied intra-tympanically, e.g., injected through the tympanic membrane into the middle ear cavity. Without being bound by any theory, this procedure is based on the premise that the drug will contact the round window membrane (RWM) of the cochlea, enter the scala tympani (ST) and spread throughout the ear. The target tissues of such treatments can include the sensory hair cells, the afferent nerve fibers and supporting cells of the cochlea (hearing) or vestibular (balance) portions of the inner ear.

Anesthetics, glucocorticoids and aminoglycosides have been used to treat inner ear disorders. Currently, the most widely used form of intratympanic therapy is the injection of glucocorticoids into the middle ear in subjects with Ménière's Disease or sudden sensorineural hearing loss. There are also clinical reports related to the local application of gentamicin for the treatment of Ménière's Disease. Gentamicin is toxic to the sensory cells of the balance system and thereby suppresses vertigo in some subjects by partially ablating their vestibular system. Other substances that have been tested in humans include local anesthetics, neurotransmitters and neurotransmitter antagonists. There is also interest in the administration of growth factors, antioxidants, apoptosis inhibitors and antisense-oligonucleotides. Animal experiments have shown promising results using locally applied drugs to provide otoprotection from noise and drug toxicity. One extension of such studies is local viral and non-viral gene transfer for the sustained treatment of inner ear disorders.

Ménière's Disease (MD) is a chronic disorder of the inner ear typically characterized by recurrent episodes of spontaneous dizziness, fluctuating hearing loss, ringing in the ears and a feeling of fullness or congestion in the ears. These clinical symptoms can have a significant negative impact on an individual's quality of life (QOL). There is no cure for MD and there are currently no approved pharmacological treatments available that are indicated for MD.

The current treatment of MD is primarily focused on decreasing the frequency and severity of vertigo attacks, reducing tinnitus and aural fullness as well as preserving or improving hearing and QOL. There are various treatments that are prescribed that include a low-salt diet, diuretics, betahistine, oral steroids, antivirals, benzodiazepines and intratympanic (IT) injections of gentamicin or corticosteroids. And in some cases, destructive surgical ablation of the cochlea or the auditory nerve can be performed.

The use of corticosteroids as a promising treatment option for MD patients stems from established clinical benefit of using corticosteroids to treat other auditory disorders, such as autoimmune inner ear disease and sudden sensorineural hearing loss (SNHL) combined with the role of inflammatory and immune mechanisms in the pathophysiology of MD. In addition to their anti-inflammatory and immunosuppressive effects in the cochlea, the mechanism of action of corticosteroids in MD has also shown to increase labyrinthine circulation and improve inner ear function through ion or water transport mechanisms influencing cochlear fluid homeostasis. The corticosteroid dexamethasone has been shown to suppress inflammation by inhibiting multiple inflammatory cytokines resulting in decreased edema, fibrin deposition, capillary leakage and migration of inflammatory cells.

There may be significant advantages to direct middle ear drug injection for managing the symptoms of MD. The tight blood-labyrinth barrier can allow therapeutic drug levels to be achieved in the inner ear following IT administration while minimizing systemic exposure and the adverse effects that are often associated with systemic administration.

The clinical use of corticosteroids to treat MD and tinnitus patients via the IT route of administration began over 30 years ago. Clinical benefit has been reported in both patient groups with no adverse reactions to the treatment. Local administration of dexamethasone, which is thought to enter the inner ear via diffusion through the round and oval window membranes, has been shown to play a role in improving hearing outcomes in patients with MD.

The AAO 2020 Clinical Practice Guideline for MD describes IT steroid therapy as a treatment option for patients with active MD not responsive to noninvasive treatment (e.g., diet and lifestyle modifications), and as an alternative to IT gentamicin therapy. Local treatment with dexamethasone sodium phosphate has been established as an option in clinical practice as a result of a systematic review and a randomized controlled trial. The conclusion is that IT steroid therapy results in more benefit than harm.

Ménière's Disease is a chronic disorder of the inner ear characterized by recurrent episodes of spontaneous dizziness (vertigo), fluctuating hearing loss, ringing in the ears (tinnitus) and a feeling of fullness or congestion in the ears (aural fullness). These symptoms can be debilitating and have a significant impact on QOL. Ménière's Disease typically presents as unilateral (affecting only one ear) with no observed difference in the ratio of right to left ear, but eventually affects the contralateral ear in 25-40% of cases (bilateral MD). Bilateral MD is associated with increased vestibular symptoms as well as an increased negative impact on health-related QOL (Espinosa-Sanchez J M and J A Lopez-Escamez. Meniere's disease. Handbook of Clinical Neurology 2016; 137: 257-77, nidcd.nih.gov/health/menieres-disease).

The prevalence of MD is approximately 50 to 200 per 100,000 adults in the United States (US) (Basura et al. Clinical Practice Guideline: Meniere's Disease. Otolaryngology—Head and Neck Surgery 2020, Vol. 162(2S) S1-S55) with a small female predominance (Lopez-Escamez J A, Carey J, Chung W H et al. (2015). Diagnostic criteria for Meniere's disease. J Vestib Res 25: 1-7). Although MD can occur at any age, it is more likely to affect adults between the 4th and 6th decade of life. As a result, children are rarely affected (Espinosa-Sanchez J M and J A Lopez-Escamez. Meniere's disease. Handbook of Clinical Neurology 2016; 137: 257-77).

There is no cure for MD and there are currently no approved pharmacological treatments available that are indicated for the treatment of MD. The natural course of MD is typically fluctuating auditory and vestibular acuity accompanied by periodic severe vertiginous spells and, long-term, progressive decline of both auditory and vestibular function (Basura et al., 2020). Some MD patients suffer from other disorders and comorbidities including allergic and autoimmune disorders (Espinosa-Sanchez J M and J A Lopez-Escamez. Meniere's disease. Handbook of Clinical Neurology 2016; 137: 257-77), though a causal relationship between MD and these comorbidities has not been well-established (Gurkov R, Pyyko I, Zou J, Kentala E. What is Meniere's disease? A contemporary re-evaluation of endolymphatic hydrops. J Neurol. 2016; 263(Suppl 1):S71-81).

In 1861, Prosper Meniere noted that vertigo, balance and hearing loss symptoms associated with MD were the result of a lesion of the inner ear (Basura et al., 2020).

Although the underlying etiology of MD is not completely clear, it has been associated with inner ear fluid (endolymph) volume increases (hydrops), culminating in episodic ear symptoms (vertigo, fluctuating hearing loss, tinnitus and aural fullness) (Basura et al., 2020).

The diagnostic criteria for MD have been jointly formulated by the Classification Committee of the Barany Society, the Japan Society for Equilibrium Research, the European Academy of Otology and Neurotology (EAONO), the Equilibrium Committee of the American Academy of Otolaryngology-Head and Neck Surgery (AAO-HNS) and the Korean Balance Society.

The classification includes two categories: definite MD and probable MD (see Table B1).

TABLE Bl
Diagnostic Criteria for Ménière's

| Disease Definite Ménière's Disease | Probable Ménière's Disease |
|---|---|
| Two or more spontaneous episodes of | Two or more spontaneous episodes of |
| vertigo, each lasting 20 minutes to 12 hours | vertigo, each lasting 20 minutes to 24 hours |
| Audiometrically documented low to | Fluctuating aural symptoms (hearing, |
| medium frequency SNHL in the affected ear | tinnitus, or fullness) in the affected ear |
| on at least one occasion before, during, or | |
| after one of the episodes of vertigo | |
| Fluctuating aural symptoms (hearing, | Not better accounted for any other |
| tinnitus, or fullness) in the affected ear | vestibular diagnosis |
| Not better accounted for any other | |
| vestibular diagnosis | |

The management of MD is generally aimed at decreasing the frequency and severity of vertigo attacks, reducing tinnitus and aural fullness, preserving or even improving hearing and improving QOL. There are various treatments that are prescribed that include a low-salt diet, diuretics, betahistine, oral steroids, antivirals, benzodiazepines and IT injections of gentamicin or corticosteroids. In the intractable cases, destructive surgical ablation of the cochlea or the auditory nerve can be performed (Albu S, Chirtes F, Trombitas V et al. (2015). Intratympanic dexamethasone versus high dosage of betahistine in the treatment of intractable unilateral Meniere disease. Am J Otolaryngol 36: 205-209; Coelho D H, Lalwani A K. Medical management of Meniere's disease. Laryngoscope 2008; 118:1099-108; Alarcón A V, Hidalgo L O, Arevalo R J, Diaz M P. Labyrinthectomy and Vestibular Neurectomy for Intractable Vertiginous Symptoms. Int Arch Otorhinolaryngol. 2017 April; 21(2) 184-190. doi:10.1055/s-0037-1599242. PMID: 28382129; PMCID: PMC5375706).

The established clinical benefit of using corticosteroids to treat other auditory disorders, such as autoimmune inner ear disease and sudden SNHL (Li H, Feng G, Wang H, Feng Y (2015). Intratympanic steroid therapy as a salvage treatment for sudden sensorineural hearing loss after failure of conventional therapy: a meta-analysis of randomized, controlled trials. Clin Ther 37: 178-187), taken together with the role of inflammatory and immune mechanisms in the pathophysiology of MD, have resulted in corticosteroids being considered as a promising treatment option in MD (Espinosa-Sanchez J M and J A Lopez-Escamez. Meniere's disease. Handbook of Clinical Neurology 2016; 137: 257-77; Lopez-Escamez J A, Vilchez J R, Soto-Varela A et al. (2007). HLA-DRB1*1101 allele may be associated with bilateral Meniere's disease in southern European population. Otol Neurotol 28: 891-895; Lopez-Escamez J A, Saenz-Lopez P, Acosta L et al. (2010). Association of a functional polymorphism of PTPN22 encoding a lymphoid protein phosphatase in bilateral Meniere's disease. Laryngoscope 120: 103-107; Hamid M, Trune D (2008). Issues, indications, and controversies regarding intratympanic steroid perfusion. Curr Opin Otolaryngol Head Neck Surg 16: 434-440; Hu A, Parnes L S. Intratympanic steroids for inner ear disorders: a review. Audiol Neurootol. 2009; 14(6):373-82).

Dexamethasone, a corticosteroid, has been shown to suppress inflammation by inhibiting multiple inflammatory cytokines resulting in decreased edema, fibrin deposition, capillary leakage and migration of inflammatory cells. In addition to their anti-inflammatory and immunosuppressive effects in the cochlea, the mechanism of action of corticosteroids in MD has also shown to increase labyrinthine circulation and improve inner ear function through ion or water transport mechanisms influencing cochlear fluid homeostasis (Espinosa-Sanchez, 2016, Nevoux J, Viengchareun S, Lema I et al. (2015). Glucocorticoids stimulate endolymphatic water reabsorption in inner ear through aquaporin 3 regulation. Pflugers Arch 467: 1931-1943).

There are significant advantages to direct middle ear drug injection for managing the symptoms of MD. The tight blood-labyrinth barrier allows therapeutic drug levels to be achieved in the inner ear following IT administration while minimizing systemic exposure.

Animal studies demonstrate that IT delivery of corticosteroids leads to significantly higher levels of steroids in the inner ear compared with systemic administration. In addition, many adverse effects associated with systemic administration can be avoided such as osteoporosis, diabetes mellitus, hypertension, peptic ulcer, cataracts, and endocrine disorders (Espinosa-Sanchez, 2016).

From a historical point of view, clinical use of corticosteroids via the IT route of administration began when Sakata et al. (1986) and ten years later when Shea et al. (Shea J J Jr, Ge X. Dexamethasone perfusion of the labyrinth plus intravenous Dexamethasone for Meniere's disease. Otolaryngol Clin North Am 1996; 29:353-358) used them to treat MD and tinnitus patients. Clinical benefit was reported in both patient groups with no adverse reactions to the treatment. Local administration of dexamethasone, which is thought to enter the inner ear via diffusion through the round and oval window membranes, has been shown to play a role in improving the hearing in patients with MD. It is thought that the dexamethasone targets the endolymphatic sac and acts on the stria vascularis and spiral ligament, which are the known targets of immune response in the inner ear. As a result, a reduction in endolymphatic hydrops (EH) is observed and fluid dynamics are restored to the endolymph (Shea et al., 1996). Following IT injection, the concentration of steroid within the perilymph has been estimated to be 260 times greater than oral administration (Devantier L, Djurhuus B D, Hougaard D D, et al. Intratympanic Steroid for Ménière's Disease: A Systematic Review. Otol Neurotol. 2019; 40(6):806-812; Bird P A, Murray D P, Zhang M, Begg E J. Intratympanic versus intravenous delivery of dexamethasone and dexamethasone sodium phosphate to cochlear perilymph. Otol Neurotol 2011; 32:933-6).

Steroid therapy via IT delivery appears to have less risk of treatment-associated hearing loss than IT gentamicin therapy, 0% to 8% versus 12.5% to 15.4%, respectively (Basura et al., 2020; Casani A P, Piaggi P, Cerchiai N, Seccia V, Franceschini S S, Dallan I. Intratympanic treatment of intractable unilateral Meniere disease: gentamicin or dexamethasone? A randomized controlled trial. Otolaryngol Head Neck Surg. 2012; 146(3):430-437; ElBeltagy Y, Shafik A, Mahmoud A, Hazaa N. Intratympanic injection in Meniere's disease; symptomatic and audiovestibular; comparative, prospective randomized 1-year control study. Egypt J Otolaryngol. 2012; 28(3):171-183; Sarafraz M, Saki N, Nikakhlagh S, Mashali L, Arad A. Comparison the efficacy of intratympanic injections of methylprednisolone and gentamicin to control vertigo in unilateral Meniere's disease. Biomed Pharmacol J. 2015; 8:705-709; Syed M I, Ilan O, Nassar J, Rutka J A. Intratympanic therapy in Meniere's syndrome or disease: up to date evidence for clinical practice. Clin Otolaryngol. 2015; 40(6):682-690). One study found a similar improvement in aural fullness with both IT steroid (38%) and IT gentamicin therapy (31%).

As in sudden hearing loss, 2 systematic reviews suggest that IT steroid therapy may have a role in salvaging hearing secondary to a MD flare (Basura et al., 2020; Lavigne P, Lavigne F, Saliba I. Intratympanic corticosteroids injections: a systematic review of literature. Eur Arch Otorhinolaryngol. 2016; 273(9):2271-2278; Patel M. Intratympanic corticosteroids in Meniere's disease: a mini-review. J Otol. 2017; 12(3):117-124), although 1 randomized controlled trial found no benefit regarding hearing salvage (Basura et al., 2020; Silverstein H, Isaacson J E, Olds M J, Rowan P T, Rosenberg S. Dexamethasone inner ear perfusion for the treatment of Meniere's disease: a prospective, randomized, double-blind, crossover trial. Am J Otol. 1998; 19(2):196-201).

When compared with placebo or with conventional medical therapy in 1 randomized controlled trial (Basura et al., 2020; Paragache G, Panda N K, Ragunathan M, Sridhara. Intratympanic dexamethasone application in Meniere's disease—is it superior to conventional therapy? Indian J Otolaryngol Head Neck Surg. 2005; 57(1):21-23) and in 3 systematic reviews (Lavigne et al., 2016; Patel, 2017; Phillips J S, Westerberg B. Intratympanic steroids for Meniere's disease or syndrome. Cochrane Database Syst Rev. 2011; (7):CD008514), IT steroid therapy generally has shown to yield greater improvement in vertigo symptoms (85%-90% vs 57%-80%). Variable benefit has been found with the associated symptoms of tinnitus and aural fullness, with 1 randomized controlled trial comparing IT steroids against placebo (Garduno-Anaya M A, Couthino De Toledo H, Hinojosa-Gonzalez R, Pane-Pianese C, Rios-Castaneda L C. Dexamethasone inner ear perfusion by intratympanic injection in unilateral Meniere's disease: a two-year prospective, placebo-controlled, double-blind, randomized trial. Otolaryngol Head Neck Surg 2005; 33:285-94) showing improvement in tinnitus (48% vs 20%), hearing loss (35% vs 10%), and fullness (48% vs 20%).

Initial work with a sustained-release form of dexamethasone has documented a reduction in vertigo frequency with 3 mg and 12 mg doses (56% and 73%, respectively) when compared with placebo (42%), with similar reductions in tinnitus (Basura et al., 2020; Lambert P R, Nguyen S, Maxwell K S, et al. A randomized, double-blind, placebo-controlled clinical study to assess safety and clinical activity of OTO-104 given as a single intratympanic injection in patients with unilateral Meniere's disease. Otol Neurotol. 2012; 33(7):1257-1265). A subsequent study reported reduced vertigo severity that was not statistically significant as compared with placebo and no difference in tinnitus perception (Basura et al., 2020; Lambert et al., 2016). Statistically significant reduction in average number of daily vertigo attacks and number of vertigo days per month was noted (Basura et al., 2020; Lambert et al., 2016). Overall, IT steroid therapy is well tolerated with low side effects and/or complications. The most frequently cited complications are post-procedure otitis media (7%) (Basura et al., 2020; Patel et al., 2016) and persistent tympanic perforation (3%-38%) (Basura et al., 2020; Lambert et al., 2012; Lambert P R, Carey J, Mikulec A A, LeBel C. Intratympanic sustained-exposure dexamethasone thermosensitive gel for symptoms of Meniere's disease: randomized phase 2b safety and efficacy trial. Otol Neurotol. 2016; 37(10):1669-1676).

As noted by Basura et al. (2020), the effectiveness of IT steroid therapy has been challenging to assess due to the variability in treatment protocols. Number of doses, time between doses, length of follow-up, and the effects on vertigo control, tinnitus, and aural fullness vary considerably (Syed M I, Ilan O, Nassar J, Rutka J A. Intratympanic therapy in Meniere's syndrome or disease: up to date evidence for clinical practice. Clin Otolaryngol. 2015; 40(6):682-690).

Steroid therapy via IT delivery may be considered an alternative for oral steroid therapy (Basura et al., 2020; Morales-Luckie E, Cornejo-Suarez A, Zaragoza-Contreras M A, Gonzalez-Perez O. Oral administration of prednisone to control refractory vertigo in Meniere's disease: a pilot study. Otol Neurotol. 2005; 26(5):1022-1026; Phillips et al., 2011; Doyle K J, Bauch C, Battista R, et al. Intratympanic steroid treatment: a review. Otol Neurotol. 2004; 25(6):1034-1039; Morgan A E, Ismail E L, Ashraf B. Intratympanic injections of dexamethasone in delayed endolymphatic hydrops: a prospective clinical study. ORL J Otorhinolaryngol Relat Spec. 2018; 80(1):19-27) and IT gentamicin therapy (Basura et al., 2020; Casani A P, Piaggi P, Cerchiai N, Seccia V, Franceschini S S, Dallan I. Intratympanic treatment of intractable unilateral Meniere disease: gentamicin or dexamethasone?A randomized controlled trial. Otolaryngol Head Neck Surg. 2012; 146(3):430-437; ElBeltagy et al., 2012; Patel et al., 2016; Sarafraz et al., 2015).

Oral steroids have significant risk of side effects (Basura et al., 2020; Stachler R J, Chandrasekhar S S, Archer S M, et al. Clinical practice guideline: sudden hearing loss. Otolaryngol Head Neck Surg. 2012; 146(3):S1-S35; Doyle et al., 2004) and patients with usable hearing may be hesitant to undergo an ablative inner ear therapy, such as IT gentamicin, with a known potential for hearing loss. Therefore, there is a significant role for patient preference when offering IT steroid therapy (Basura et al., 2020; Radtke A, Lempert T, Gresty M A, Brookes G B, Bronstein A M, Neuhauser H. Migraine and Meniere's disease: is there a link? Neurology. 2002; 59(11):1700-1704). Intratympanic delivery can be a minimally invasive injection performed in the office setting that offers a potential direct route of administration (Piu F, Wang X, Fernandez R, Dellamary L, Harrop A, Ye Q, Sweet J, Tapp R, Dolan D F, Altschuler R A, Lichter J, LeBel C. OTO-104: a sustained-release dexamethasone hydrogel for the treatment of otic disorders. Otol Neurotol. 2011 January; 32(1):171-9).

Over the years, there have been several clinical investigations evaluating the safety and efficacy of IT administration of corticosteroids in MD patients. The safety, tolerability and clinical activity of a single IT injection of 12 mg of dexamethasone (n=93) was evaluated in two recent clinical studies in patients with unilateral MD. Results demonstrated that dexamethasone (formulated in a buffered solution containing a glycol polymer, poloxamer 407) was safe, well-tolerated and showed promising improvement in vertigo endpoints supportive of moving this program into a Phase 3 which is currently ongoing. (Lambert P R, Nguyen S, Maxwell K S, et al. A randomized, double-blind, placebo-controlled clinical study to assess safety and clinical activity of OTO-104 given as a single intratympanic injection in patients with unilateral Meniere's disease. Otol Neurotol. 2012; 33(7):1257-1265; Lambert P R, Carey J, Mikulec A A, LeBel C. Intratympanic sustained-exposure dexamethasone thermosensitive gel for symptoms of Meniere's disease: randomized phase 2b safety and efficacy trial. Otol Neurotol. 2016; 37(10):1669-1676).

In a Phase 1b study, 16 patients received a single IT injection of 12 mg dexamethasone, 14 patients received 3 mg dexamethasone and 14 received placebo. There were no deaths, no serious adverse events (SAEs) and no adverse events (AEs) leading to discontinuation from this study. There were also no adverse findings in laboratory measurements, physical examination, vital signs or electrocardiogram (ECG) (Lambert et al., 2012).

In this study, most patients had at least 1 treatment-emergent adverse event (TEAE). The only prespecified AE of interest that was observed in more than 1 patient was perforation of the tympanic membrane (TM). At the end of this study, the incidence of TM perforation was 3% in patients who received dexamethasone (either 3 mg or 12 mg). Perforation of the TM has been observed in other studies using IT injection (Lambert et al., 2012; Rauch S D, Halpin C F, Antonelli P J, et al. Oral vs intratympanic corticosteroid therapy for idiopathic sudden sensorineural hearing loss: a randomized trial. JAMA 2011; 305:2071-79; Herraiz C, Plaza G, Aparicio J M, et al. Transtympanic steroids for Meniere's disease. Otol Neurotol 2010; 31:162-7) and most perforations resolved spontaneously (Lambert et al., 2012; Muehlmeier G, Biesinger E, Maier H. Safety of intratympanic injection of AM-101 in patients with acute inner ear tinnitus. Audiol Neurotol 2011; 16:388-97).

In a Phase 2b study, 77 patients received a single IT injection of 12 mg dexamethasone and 77 patients received placebo. Most AEs were mild or moderate in severity and no TEAE resulted in patient discontinuation in this study. Results of the safety assessments in this study (otoscopy, audiometry, tympanometry, vital signs, clinical laboratory evaluations, word recognition and the Columbia-Suicide Rating Scale (C-SSRS)) support continued evaluation of IT injection of corticosteroids as they appear to be well-tolerated; no new risks were identified. Persistent TM perforations were observed in two dexamethasone-treated patients at the end of this study which is consistent with perforations observed following IT administration of a corticosteroid (Lambert et al., 2016; Garduno-Anaya M A, Couthino De Toledo H, Hinojosa-Gonzalez R, Pane-Pianese C, Rios-Castaneda LC. Dexamethasone inner ear perfusion by intratympanic injection in unilateral Meniere's disease: a two-year prospective, placebo-controlled, double-blind, randomized trial. Otolaryngol Head Neck Surg 2005; 33:285-94; Silverstein H, Farrugia M, Van Ess M. Dexamethasone inner ear perfusion for subclinical endolymphatic hydrops. Ear Nose Throat J 2009; 88:778-85; Kitahara T, Kubo T, Okumura S, Kitahara M. Effects of endolymphatic sac drainage with steroids for intractable Meniere's disease: a long-term follow-up and randomized controlled study. Laryngoscope 2008; 118:854-61; Herraiz C, Plaza G, Aparicio J M, et al. Transtympanic steroids for Meniere's disease. Otol Neurotol 2010; 31:162-7; Rauch S D, Halpin C F, Antonelli P J, et al. Oral vs intratympanic corticosteroid therapy for idiopathic sudden sensorineural hearing loss: a randomized trial. JAMA 2011; 305:2071-79).

EH is generally accepted as the pathologic hallmark of MD although the etiology of the disease remains unclear.

In 2010, Naganawa et al. (Naganawa, S. et al. Visualization of endolymphatic hydrops in Meniere's disease with single-dose intravenous gadolinium-based contrast media using heavily T(2)-weighted 3D-FLAIR. Magn Reson Med Sci 9, 237-242 (2010)) developed an IV-gadolinium (Gd) enhanced inner ear MRI which visualized EH in patients with MD. The IV-Gd enhanced inner ear MRI is minimally invasive, has a relatively short waiting time (4 hours) and can visualize both inner ears simultaneously which enables identification of asymptomatic EH in the opposite ear. Cho Y S et al. (Cho Y S, Ahn J M, Choi J E, et al. Usefulness of Intravenous Gadolinium Inner Ear MR Imaging in Diagnosis of Ménière's Disease. Sci Rep. 2018; 8(1):17562. Published 2018 Dec. 3. doi:10.1038/s41598-018-35709-5) conducted a clinical study that aimed to investigate the usefulness of the IV-Gd enhanced inner ear MRI in diagnosing MD to find a correlation between the degree of EH and the audiovestibular tests. The results demonstrate appropriate correlations with auditory vestibular functional testing which show the usefulness of IV-Gd inner ear MRI as a diagnostic method for visualizing the EH in MD. Despite these findings, EH is currently not part of the diagnostic criteria for definite MD.

To improve the diagnostic accuracy in patients with suspected MD based on imaging, recent studies have introduced perilymphatic enhancement (PE) as an additional MD-discriminating parameter. What remains unclear, however, is the presence and value of PE in other vertigo-associated inner ear pathology (VAIEP).

In February 2020, J M van Steekelenburg et al published a retrospective analysis of 220 patients (median age, 55.8) with inner ear pathology, suspected of having MD. The purpose of this study was to evaluate the presence of EH and the additional value of PE in the diagnosis of patients with MD and in patients with other VAIEP not attributable to MD (Van Steekelenburg J M, van Weijnen A, de Pont L M H, Vijlbrief O D, Bommelje C C, Koopman J P, Verbist B M, Blom H M, Hammer S. Value of Endolymphatic Hydrops and Perilymph Signal Intensity in Suspected Meniere Disease. AJNR Am J Neuroradiol. 2020 March; 41(3):529-534. doi: 10.3174/ajnr.A6410. Epub 2020 Feb. 6). The results showed that increased PE was more prevalent in definite and probable MD ears compared with other VAIEP ears (p<0.001 and p=0.003, respectively) and asymptomatic ears (both, p<0.001). Since vestibular or cochlear EH or both were present in 91.9% of the definite MD ears, this study emphasizes the relevance of EH as a hallmark of definite MD. Compared with asymptomatic ears, the definite MD ears showed increased PE both visually and in quantitative measurements.

This study also demonstrates the value of delayed Gd enhanced 3D-FLAIR MRI in diagnosing MD in a cohort with a wide range of VAIEP showing that the combination of EH and increased PE is uncommon in patients with other VAIEP. These findings have the potential to help differentiate patients with vertigo attributable to MD (van Steekelenburg et al, 2020).

Off-label IT steroid injections are often administered in MD patients. However, the therapeutic benefit is limited, at least in part, by rapid clearance of solution formulations from the middle ear, uncertain middle ear placement of drug formulations because the injections are “blind”, and the presence of membranous barriers and air pockets in the middle ear. Current MD treatment guidelines (Basura, 2020), suggest compounded dexamethasone sodium phosphate or methylprednisolone sodium succinate solutions be administered in 3 to 4 sessions every 3 to 7 days. Suspension gel formulations of dexamethasone have shown promise in the amelioration of vertigo symptoms in MD patients (Lambert 2012, 2016), but even such thermoresponsive gel formulations have been shown to clear from the middle ear within days after administration (Piu 2011). Pseudomembranes (false membranes) are present in 42% of ears (Şahin B, Orhan K S, Aslιyüksek H, Kara E, Büyük Y, Güldiken Y. Endoscopic evaluation of middle ear anatomic variations in autopsy series: analyses of 204 ears. Braz J Otorhinolaryngol. 2020 January-February; 86(1):74-82), limiting the contact with the round window membrane of rapidly clearing and randomly placed formulations.

However, currently available formulations for intra-tympanic delivery have short middle ear residence times and usually require multiple administrations to achieve the desired effects in the inner ear. The short residence of these formulations may result in a lack of uniform drug distribution and release, with poor pharmacokinetics.

Therefore, it is an object to provide formulations with beneficial effects that can be administered for sustained intra-tympanic delivery of therapeutic, prophylactic, or diagnostic agents over a period of days into the inner ear, providing controlled release and pharmacokinetics while minimizing risk of systemic exposure and reducing the need for repeated administrations.

### I. Definitions

“Active agent” and “active pharmaceutical ingredient” are used interchangeably and refer to a physiologically and/or pharmacologically active substance that acts locally and/or systemically in the body. An active agent is a substance that is administered to a subject for the treatment (e.g., glucocorticoids or antiangiogenics), prevention (e.g. antiapoptotics), or diagnosis (e.g. gadolinium) of a disease or disorder.

The term “AUC” or “area under the curve” in the field of pharmacokinetics is the definite integral in a plot of active agent concentration in blood plasma versus time. In practice, the active agent concentration is typically measured at certain discrete points in time and the trapezoidal rule is used to estimate AUC. The AUC of an active agent is typically used to evaluate the exposure of a subject to an active agent over time.

The term “auditory brainstem response” or “ABR” refers to an auditory evoked potential extracted from ongoing electrical activity in the brain and recorded via electrodes placed on, for example, the scalp. ABR is considered an exogenous response because it is dependent on external factors.

The term “blood labyrinth barrier” or “BLB” refers to the barrier between the vasculature and the inner ear fluids, either endolymph or perilymph. The BLB is involved in the maintenance of the inner ear fluid ionic homeostasis.

The term “BLLQ” is an abbreviation for “below the lower limit of quantification” and is defined as below the lowest standard on a calibration curve.

The term “Cₘₐₓ” refers to the maximum (or peak) serum concentration that an active agent achieves (e.g., systemically, or in a specified compartment or test area of the subject) after the active agent has been administered. In some embodiments, Cₘₐₓ is measured before the administration of a second dose of the active agent.

The term “Cₘᵢₙ” refers to the minimum (or trough) serum concentration that an active agent achieves (e.g., systemically, or in a specified compartment or test area of the subject) after the active agent has been administered. In some embodiments, Cₘᵢₙ is measured before the administration of a second dose of the active agent.

The term “cytocochleogram” refers to a graphic representation of the anatomical state of the hair cells along the complete width and length of the organ of Corti.

The abbreviation “DDI” refers to drug-drug interaction.

The term “degree of functionalization” when referring to polymers that can participate in crosslinking, is the number of functional groups per appropriate polymeric unit (e.g., polymer chain, branch, or monomer) that are suitable for crosslinking using a given crosslinker. For example, if a polymer has one or more functional group(s) per monomer, then the appropriate polymeric unit is a monomer. As another example, if a polymer has one or more functional group(s) per branch terminus, then the appropriate polymeric group is a branch. It will be further understood that a degree of functionalization, in some cases, can be less than one, for example, if a subpopulation of the functional groups have degraded.

The term “drug absorption” or “absorption” refers, typically, to the process of movement of the active agent from the localized site of administration to a site of therapeutic effect. In some cases, drug absorption can be through the round window niche of the cochlea, and across a barrier (e.g., the round window membrane) into one or more inner ear structures. The term “co-administration”, as used herein, are meant to encompass, generally, administration of two or more active agents to a single subject, and are intended to include prevention regimens in which the active agents are administered by the same or different route of administration or at the same or different time.

The term “elastic”, as used herein with reference to a gel, can mean that the gel demonstrates elasticity, e.g., resisting a distorting force and returning to its original size and shape when the force is removed. An elastic modulus can be measured, in some cases, by oscillatory rheology.

The phrase “effective amount” or “effective concentration” means an amount of active agent that, when at a site of action, is sufficient to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein. The amount of an active agent that will correspond to such an amount will vary depending upon factors such as the particular active agent, disease condition and its severity, the identity (e.g., age and/or weight) of the patient in need of treatment, but can nevertheless be routinely determined by one skilled in the art. The terms “effective amount” or “therapeutically effective amount,” as used herein, can refer to a sufficient amount an active agent at a site of action that would be expected to relieve to some extent one or more of the symptoms of the disease or condition being treated. In some embodiments, an effective amount of an active agent is a quantity necessary to render a desired anti-inflammatory result at a site of action. The term “therapeutically effective amount” includes, for example, an “effective amount” of an active agent to achieve a desired pharmacologic effect without undue adverse side effects.

The phrase “effective dose” means an amount of active agent that, when administered to a patient in need of such treatment, is sufficient to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein. In some embodiments, an “effective dose” is an amount of active agent, when administered to a patient in need of such treatment, achieves a sufficient concentration at a site of action to (i) treat a disease or disorder, (ii) attenuate, ameliorate, or eliminate one or more symptoms of the particular disease, condition, or disorder, or (iii) delay the onset of one or more symptoms of the particular disease, condition, or disorder described herein for a period of time. The dose of an active agent that will correspond to such an amount will vary depending upon factors such as the particular active agent, disease condition and its severity, the identity (e.g., age and/or weight) of the patient in need of treatment, but can nevertheless be routinely determined by one skilled in the art. The terms “effective dose” or “therapeutically effective dose,” as used herein, can refer to a sufficient amount of an active agent being administered that would be expected to relieve to some extent one or more of the symptoms of the disease or condition being treated. In some embodiments, an effective dose of an active agent is a quantity necessary to render a desired anti-inflammatory result. The term “therapeutically effective dose” includes, for example, an “effective dose” of an active agent to achieve a desired pharmacologic effect without undue adverse side effects. It will also be understood that “an effective dose” in an extended-release dosing format may differ from “an effective dose” in an immediate-release dosing format based upon pharmacokinetic and/or pharmacodynamic considerations.

The term “enhance” or “enhancing,” can refer to an increase in potency or a prolongation of a desired effect. In some cases, “enhance” or “enhancing” can also refer to a decrease of one or more adverse effects associated with an active agent. For example, in reference to enhancing the effect of the active agents disclosed herein, the term “enhancing” can refer to the ability to increase the potency or prolong the duration of effect of the active agent by an anti-inflammatory agent. An “enhancing-effective amount,” as used herein, refers to an amount of an agent that is adequate to enhance the effect of an active agent in a desired system. The amount of an agent that will correspond to such an amount will vary depending upon factors such as the particular active agent, disease condition and its severity, the identity (e.g., age and/or weight) of the patient in need of treatment, but can nevertheless be routinely determined by one skilled in the art.

The term “a gel” refers to a semisolid composition. In some embodiments, a gel can be differentiated from a liquid by assessing flow under gravity, for example, by performing a gelation reaction in a vial, then inverting the vial. In some assessments, a visual inspection can confirm whether there is still flow of the composition. In some cases, a gravimetric assessment can be performed after inverting a vial and wiping off liquid from a sample in an insert. In some cases, a gel can be differentiated from a liquid by the ability of the composition to prevent a stir bar from spinning (e.g., a 7×2 mm PTFE stir rod with about 0.2 to about 1 mL of a composition in a 2 mL vial). In some embodiments, a gel can be differentiated from a liquid by having an elastic modulus. In some embodiments, formation of a gel can be determined by a rapid change in the ordinate value when plotting the ratio of storage modulus to loss modulus (G″/G′) vs time (t). In some embodiments, a gel can be differentiated from a liquid by analyzing its cohesion, for example, by drop weight or compression force (see, e.g., Edsman, Katarina L M, et al. “Is there a method that can measure cohesivity? Cohesion by sensory evaluation compared with other test methods.” Dermatologic Surgery 41 (2015): S365-S372 and Edsman, Katarina L M, and Ake Ohrlund. “Cohesion of hyaluronic acid fillers: correlation between cohesion and other physicochemical properties.” Dermatologic Surgery 44.4 (2018): 557, both of which are incorporated by reference herein in their entireties).

The term “to gel” refers to the formation of a gel. Typically, a gel is formed by gelation of a liquid composition.

The term “gel duration” refers to the amount of time a gel lasts before being degraded, dissolved, or turning back into a solution. In some cases, gel duration is measured by placing a gel into a vial and storing at room temperature, at 37° C., or at an accelerated condition of 50° C. In some cases, gel duration is measured by placing a gel (e.g. at least 1 mL) in a receptor solution (e.g., pH 7.4 PBS) at 37° C. (or at an accelerated condition of 50° C.), optionally periodically changing the receptor solution.

The term “gelation time” refers to the amount of time it takes, after combining all appropriate components, for a composition to form a gel. In some cases, the gelation time can be determined by measuring the time it takes to achieve one or more of the properties of a gel as defined herein. In some embodiments, the gelation time can be determined by measuring the time it takes for a stir bar to stop spinning in a container in which gelation occurs.

The term “GLP” refers to “good laboratory practice” and is a set of principles intended to assure the quality and integrity of non-clinical laboratory studies.

The term “hERG” can refer to a human ether-a-go-go-related gene that encode a protein that is the alpha subunit of a potassium ion channel. In some cases, the ion channel that includes this subunit is also called hERG.

The term “IC₅₀” refers to the concentration of an inhibitor at which an assayed outcome is reduced by 50%.

The term “inhibit” can mean to reduce or decrease an activity (e.g., signaling activity) or expression (e.g., of a gene or gene product). The term can also include preventing, slowing, or reversing the development of a disease or condition or the advancement of a disease or condition in a subject. In some cases, inhibition can be partial. In some cases, inhibition can be complete. In some embodiments, a level of inhibition can be determined based on comparison to a control or to a standard level.

The term “macromolecule” generally refers to a molecule that greater than 1500 g/mol, greater than about 2000 g/mol, or greater than about 2500 g/mol. In some forms, a macromolecule can be polymeric and/or oligomeric.

The term “MRSD” or “maximum recommended starting dose” refers to the highest amount of an agent that can be given safely and without complication while maintaining its efficacy.

The term “MTD” or “maximum tolerated dose” refers to the highest dose of a drug or prevention that does not cause unacceptable side effects.

The term “mucoadhesion” as used herein, can refer to adhesion between two materials, one of which is a mucosal surface.

The term “NOAEL” refers to “no observed adverse effect level” and can be an important part of the non-clinical risk assessment.

The terms “otic” and “auris” refer to relating to the ear. For example, an otic composition can be a composition intended for administration to the ear.

The term “pharmaceutically acceptable” indicates that the compound, or salt or composition thereof is compatible chemically and/or toxicologically with the other ingredients comprising a formulation and/or the patient being treated therewith. In some embodiments, a pharmaceutically acceptable salt can be a salt that conserves the efficiency and/or the biological properties of the free bases or free acids. In some embodiments, a pharmaceutically acceptable salt can be a salt that change the efficiency and/or the biological properties of the free bases or free acids; for example, a pharmaceutically acceptable salt can improve the bioavailability of a free base or free acid.

The term “pharmaceutical combination”, as used herein, refers to a pharmaceutical therapy resulting from the mixing or combining of more than one active agent and includes both fixed and non-fixed combinations of the active agents. The term “fixed combination” means that a first active agent or a pharmaceutically acceptable salt or solvate thereof and at least one additional active agent, are both administered to a patient simultaneously in the form of a single composition or dosage. The term “non-fixed combination” means that a first active agent or a pharmaceutically acceptable salt or solvate thereof and at least one additional active agent are formulated as separate compositions or dosages, such that they may be administered to a subject in need thereof simultaneously, concurrently or sequentially with variable intervening time limits, using the same or different routes of administration, wherein such administration provides effective levels of the two or more compounds in the body of the subject. In one embodiment, the first active agent and the second active agent are formulated as separate unit dosage forms, wherein the separate dosage forms are suitable for either sequential or simultaneous administration. These also apply to cocktail therapies, e.g., the administration of three or more active ingredients.

The term “pot life”, as used herein when referring to a solution or suspension containing moiety that includes an electrophile that can form crosslinks with a nucleophile (e.g., a succinimidyl ester-functionalized PEG), refers to the time since the moiety was made into the solution or suspension (e.g., from a powder or a solid).

The term “VEGF inhibitor” includes any agent (e.g., a small molecule, antibody, or antigen-binding fragment thereof) exhibiting inhibition of vascular endothelial growth factor (VEGF) signaling. In some embodiments, a VEGF inhibitor can bind to a vascular endothelial growth factor receptor (a VEGFR). In some embodiments, a VEGF inhibitor can bind to a vascular endothelial growth factor (e.g., a ligand of a VEGFR).

The term “auris-acceptable penetration enhancer” or “penetration enhancer” refers to an agent that reduces barrier resistance (e.g., barrier resistance of the round window membrane).

The term “prophylactically effective amount” means an amount of active agent that, when administered to a patient in need of such treatment and at a site of action, is sufficient to (i) prevent a disease or disorder, (ii) attenuate, ameliorate, or eliminate one or more symptoms of the particular disease, condition, or disorder, before it occurs. In some cases, a “prophylactically effective amount” refers to an amount of a composition at a site of action, having been administered to a subject susceptible to or otherwise at risk of a particular disease, disorder or condition, for example, a prophylactically effective amount of an active agent can be an amount effective to prevent or to attenuate ototoxicity at a site of action.

The term “prophylactically effective dose” means an amount of active agent that, when administered to a patient in need of such treatment, is sufficient to (i) prevent a disease or disorder, (ii) attenuate, ameliorate, or eliminate one or more symptoms of the particular disease, condition, or disorder, before it occurs. In some cases, a “prophylactically effective dose” refers to an amount of a composition administered to a subject susceptible to or otherwise at risk of a particular disease, disorder or condition, for example, a prophylactically effective amount of an active agent can be an amount effective to prevent or to attenuate ototoxicity. For example, an apoptotic inhibitory formulation may be administered to an individual prior to chemotherapy to prevent hearing loss by a subsequently administered chemotherapeutic agent.

The term “residence time” as used herein can refer to the amount of time that a formulation remains in the location of administration. In some embodiments, residence time can be the time when there is no gel visualized on the round window membrane area, e.g., after collecting the gel at a time after injection.

The term “room temperature” refers to a temperature between about 15° C. and less than about 27° C., preferably about 20° C.

The term “body temperature” refers to a temperature between about 36.5° C. and about 37.5° C., preferably about 37° C.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented.

“Small molecule” generally refers to a molecule that is less than about 1500 g/mol, less than about 1000 g/mol, less than about 800 g/mol, or less than about 500 g/mol. In some forms, small molecules are non-polymeric and/or non-oligomeric. In some embodiments, a small molecule can be organic. In some embodiments, a small molecule can be inorganic. In some embodiments, a small molecule can include both organic and inorganic atoms.

“Steady state,” can refer to when the amount of drug administered (e.g., auris media and/or auris interna) is equal to the amount of drug eliminated within one dosing interval resulting in a plateau or constant levels of drug exposure within the targeted structure.

“Stable” as used herein can refer to chemical and/or physical stability over a time period under defined conditions. In some embodiments, a stable solution can retain a high percentage or all of what was originally dissolved remaining in solution. In some embodiments, a solution can retain more than 60, 70, 80, 90, 95, 98, 99, or 100% of the originally dissolved solute at room temperature (approximately 15-25° C., most preferably 25° C.).

“Sustained release” as used herein refers to release of a substance over an extended period of time. In some embodiments, this can be contrasted with to a bolus type administration in which the entire amount of the substance is made biologically available at one time.

“Swelling” of a gel as used herein can refer to a percent increase in gel weight after equilibration with phosphate-buffered saline (PBS). Swelling of a gel can be measured, for example, by preparing a gel inside an insert, recording initial gel weight, allowing gel to form at room temperature for about 20 to 60 minutes, submerging in PBS (PBS volume at least 5× volume of gel) (e.g., pH 7.4) and storing at 37° C., removing the gel-filled insert after 1-3 days, and recording weight after wiping off surface fluid, followed by a calculation of the increase in gel weight normalized by the initial gel weight.

The term “Tₘₐₓ” refers to the time it takes a drug or other substance to reach the maximum concentration Cₘₐₓ.

The term “transtympanic” or “intratympanic” administration refers to the administration of an active agent via the tympanic cavity, in some cases, via a hypodermic needle that accesses the tympanic cavity (middle ear) by penetrating the tympanic membrane (eardrum).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable.

The term “unit dosage form” refers to physically discrete units suitable as unitary dosages for human subjects and other patients, each unit containing a predetermined quantity of active material (i.e., an active agent as provided herein) calculated to produce the desired therapeutic effect, in association with a suitable pharmaceutical excipient.

The terms “prevent,” “preventing” or “prevention,” as used herein means the prevention of the onset, recurrence or spread, in whole or in part, of a disease or condition as described herein, or a symptom thereof.

### II. Extended Release Otic Compositions

Provided herein are otic compositions (e.g., an extended release otic composition) comprising a polymer composition as provided herein and an active agent. Otic (sometimes also called auris) compositions have been developed for extended release, either continuously or in a pulsatile manner, or variants of both, of therapeutic, prophylactic and/or diagnostic agent(s) within the ear. In some embodiments, an extended release otic composition as described herein can increase the area under the curve (AUC) of the agent being delivered in otic fluids (e.g., endolymph and/or perilymph) by about 30%, about 40%, about 50%, about 60%, about 70%, about 80% or about 90% compared to a composition that is not an extended release otic composition. The extended release compositions can, in some cases, also decrease the Cₘₐₓ in otic fluids (e.g., endolymph and/or perilymph) by about 40%, about 30%, about 20%, or about 10%, compared to a composition that is not an extended release otic composition. This can reduce the ratio of Cₘₐₓ to Cₘᵢₙ compared to a composition that is not an extended release otic composition. Therefore, in some embodiments, an extended release otic composition can provide a more constant release of an active agent. In certain implementations, the ratio of Cₘₐₓ to Cₘᵢₙ can be 10:1, 9:1, 8:1, 7:1, 6:1, 5:1, 4:1, 3:1, 2:1 or 1:1. The length of time that the concentration of an otic agent is above Cₘᵢₙ can be increased by about 30%, about 40%, about 50%, about 60%, about 70%, about 80% or about 90% compared to a composition that is not an extended release otic composition. In certain instances, an extended release composition can delay the time to Cₘₐₓ, and/or prolong the time the concentration of the drug will stay above the Cₘᵢₙ. In some forms, extended release otic compositions prolong the residence time of a drug in the inner ear.

In some instances, sustained delivery to the middle and/or inner ear can be achieved because the extended release otic composition is provided in the form of a gel. In some such embodiments, the gel can remain intact at a preferred location, such as the round window membrane, for extended periods of time. In some embodiments, the extended release otic composition in the form of a gel can extend residence times by at least about two-fold, four-fold, ten-fold, or twenty-fold and this can lead to increases of AUC by about two-fold, four-fold, ten-fold, or twenty-fold compared to a composition that is not an extended release otic composition.

In some embodiments, once the concentration in the endolymph or perilymph of a drug reaches steady state, the concentration of the active agent in the endolymph or perilymph stays at or about an effective concentration for an extended period of time (e.g., one day, 2 days, 3 days, 4 days, 5 days, 6 days, 1 week, 2 weeks, 3 weeks, 1 month, 2 months, 3 months, 4 months, 5 months, or 6 months).

In some embodiments, extended release otic compositions can have at least two components: an active agent (e.g., a therapeutic, prophylactic and/or diagnostic agent); a gel forming polymer composition (e.g., including a functional polymer, a crosslinker, and water); and optionally one or more excipients, which together form an extended release otic composition.

### A. Therapeutic, Prophylactic and Diagnostic Agents

In some embodiments, extended release otic compositions are useful for localized therapy, prophylaxis, and/or diagnosis. A therapeutically active, prophylactically active, diagnostic or visualization agent, or combination thereof can be delivered from the extended release otic composition, for instance, from a crosslinked polymer or gel formed following administration of the extended release otic composition.

An active agent can be any appropriate active agent. In some embodiments, an active agent can be a therapeutic agent. In some embodiments, an active agent can be a prophylactic agent. In some embodiments, an active agent can be a diagnostic or visualization agent. In some embodiments, an active agent can include a diagnostic or visualization agent and a therapeutic agent. In some embodiments, an active agent can include a diagnostic or visualization agent and a prophylactic agent. An active agent can include, for example, a protein (e.g., an enzyme, a growth factor, an antibody or an antigen-binding fragment thereof), a carbohydrate (e.g., a glycosaminoglycan), a nucleic acid (e.g., an antisense oligonucleotide, an aptamer, a micro RNA, a short interfering RNA, or a ribozyme), small molecules, or combinations thereof. In some embodiments, a small molecule can include an antibiotic, an antineoplastic agent (e.g., doxorubicin), a local anesthetic, a steroid, a hormone, an apoptotic inhibitor (for example, an inhibitor of Apaf-1; see, e.g., U.S. Pat. No. 9,040,701, incorporated by reference herein in its entirety), an angiogenic agent, an anti-angiogenic agent (e.g., a VEGF inhibitor), a neurotransmitter, a psychoactive drug, an anti-inflammatory, and combinations thereof. In some embodiments, an active agent can include an anti-angiogenic agent. In some embodiments, an active agent can include an anti-angiogenic agent and a steroid. In some embodiments, an active agent includes dexamethasone. In some embodiments, an active agent is dexamethasone.

In some embodiments, a diagnostic or visualization agent can include a dye, a fluorophore, an MRI contrast agent (e.g., an agent including gadolinium) or other agents detectable by ultrasound, MRI, or x-ray. In some embodiments, a visualization agent can improve visibility of a polymer composition or extended release otic composition during a surgical procedure. Non-limiting examples of visualization agents can include colored substances suitable for use in medical implantable medical devices, such as FD&C dyes 1, 3, and 6, eosin, methylene blue, indocyanine green, or colored dyes normally found in synthetic surgical sutures. In some embodiments, a visualization agent can include FD&C Blue #1. In some embodiments, a visualization agent can be present in a polymer composition or extended release otic composition in an amount of about 0% to about 0.5% (e.g., about 0% to about 0.02%, about 0% to about 0.05%, about 0% to about 0.1%, about 0% to about 0.2%, about 0.02% to about 0.5%, about 0.05% to about 0.5%, about 0.1% to about 0.5%, or about 0.2% to about 0.5%) by weight. In some embodiments, a visualization agent can be present in a polymer composition or extended release otic composition in an amount of about 0% to about 0.05% (e.g., about 0.005% to about 0.02%, about 0.005% to about 0.0015%, about 0.009%, or about 0.1%) by weight. In some embodiments, a visualization agent is green or blue; without being bound by any particular theory, green or blue may have better visibility in the presence of blood or on a pink or white tissue background.

In some embodiments, the active agent can be substantially in the form of microparticles or nanoparticles. Without being bound by any particular theory, it is believed that in some cases, a microparticle form can aid in the controlled release of an active agent from an extended release otic composition as described herein. In some cases, a nanoparticle form can increase dissolution rates and deliver active agent at controlled release rates higher than from microparticles. In some embodiments, a microparticle can be a particle that is between about 0.1 μm and about 100 μm in diameter (e.g., between about 0.1 μm and about 1 μm, between about 0.1 μm and about 10 μm, between about 0.1 μm and about 50 μm, between about 1 μm and about 100 μm, between about 10 μm and about 100 μm, between about 50 μm and about 100 μm, between about 1 μm and about 50 μm, or between about 1 μm and 10 μm), for instance, as measured by optical microscopy. In some embodiments, a nanoparticle can be a particle that is between about 1 nm and about 100 nm in diameter (e.g., between about 1 nm and about 10 nm, between about 1 nm and about 50 nm, between about 10 nm and about 100 nm, or between about 50 nm and about 100 nm), for instance, as measured by electron microscopy.

In some embodiments, an active agent can include an anti-angiogenic agent. In some embodiments, an anti-angiogenic agent can be a VEGF inhibitor. In some cases, a VEGF inhibitor can be an antibody or an antigen-binding fragment thereof, a decoy receptor, a VEGFR kinase inhibitor, an allosteric modulator of a VEGFR, or a combination thereof. In some cases, a VEGF inhibitor can be an antibody or an antigen-binding fragment thereof. For example, in some embodiments, a VEGF inhibitor can be alacizumab, bevacizumab (AVASTIN®), icrucumab (IMC-18F1), ramucirumab (LY3009806, IMC-1121B, CYRAMZA®), or ranibizumab (LUCENTIS®). In some embodiments, a VEGF inhibitor can be a decoy receptor (e.g., aflibercept). In some embodiments, a VEGF inhibitor can be a VEGFR kinase inhibitor, such as agerafenib, altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, or vandetanib. Other examples of VEGF inhibitors may be known in the art. In some embodiments, a VEGFR inhibitor can be an allosteric modulator of a VEGFR (e.g., cyclotraxin B).

In some embodiments, a VEGF inhibitor can be selective for VEGFR2 over other VEGFRs. In some embodiments, VEGF inhibitor can exhibit at least a 10-fold selectivity for VEGFR2 over another VEGFR. For example, VEGF inhibitor can exhibit at least a 20-fold selectivity, at least a 30-fold selectivity, at least a 40-fold selectivity; at least a 50-fold selectivity; at least a 60-fold selectivity; at least a 70-fold selectivity; at least a 80-fold selectivity; at least a 90-fold selectivity; at least 100-fold selectivity; at least 200-fold selectivity; at least 300-fold selectivity; at least 400-fold selectivity; at least 500-fold selectivity; at least 600-fold selectivity; at least 700-fold selectivity; at least 800-fold selectivity; at least 900-fold selectivity; or at least 1000-fold selectivity for VEGFR2 over another VEGFR. In some embodiments, selectivity for VEGFR2 over another VEGFR is measured in an enzyme assay. In some embodiments, another VEGFR can be selected from the group consisting of VEGFR1, VEGFR3, and both VEGFR1 and VEGFR3.

The vascular endothelial growth factor (VEGF) signaling pathway has been linked to a number of diseases and disorders, including cancer, rheumatoid arthritis, and age-related macular degeneration. In general, activation of the VEGF signaling pathway typically results in angiogenesis of a tissue. The VEGF signaling pathway transmits signal through its constituent four receptors: VEGFR1 (also called Flt-1; an exemplary human VEGFR1 sequence is shown in SEQ ID NO: 1), VEGFR2 (also called KDR or Flk-1; an exemplary human VEGFR2 sequence is shown in SEQ ID NO:2), and VEGFR3 (also called Flt4; an exemplary human VEGFR3 sequence is shown in SEQ ID NO:3), and its five growth factors: VEGF-A (an exemplary human VEGF-A sequence is shown in SEQ ID NO:4), VEGF-B (an exemplary human VEGF-B sequence is shown in SEQ ID NO: 5), VEGF-C (an exemplary human VEGF-C sequence is shown in SEQ ID NO: 6), VEGF-D (an exemplary human VEGF-D sequence is shown in SEQ ID NO: 7), and PlGF (placental growth factor; an exemplary human PlGF sequence is shown in SEQ ID NO: 8). The VEGFs have different affinities for the various VEGFRs; see, e.g., Shibuya, Masabumi. “VEGF-VEGFR signals in health and disease.” Biomolecules & Therapeutics 22.1 (2014): 1, doi: 10.4062/biomolther.2013.113, incorporated herein by reference in its entirety). Both the VEGFs and the VEGFRs have variant isoforms and/or may be processed into a mature form as compared to the sequences shown herein. VEGF-A, in particular, has several isoforms in humans. The VEGFRs can typically be spliced as soluble or membrane-bound forms. In some accounts, the VEGFRs are grouped with the platelet-derived growth factor receptors (PDGFRs) as a superfamily of tyrosine kinase receptors.

human VEGFR1 sequence from Uniparc
ID UPI000013DCDD,
SEQ ID NO: 1
MVSYWDTGVL LCALLSCLLL TGSSSGSKLK DPELSLKGTQ
HIMQAGQTLH LQCRGEAAHK WSLPEMVSKE SERLSITKSA
CGRNGKQFCS TLTLNTAQAN HTGFYSCKYL AVPTSKKKET
ESAIYIFISD TGRPFVEMYS EIPEIIHMTE GRELVIPCRV
TSPNITVTLK KFPLDTLIPD GKRIIWDSRK GFIISNATYK
EIGLLTCEAT VNGHLYKTNY LTHRQTNTII DVQISTPRPV
KLLRGHTLVL NCTATTPLNT RVQMTWSYPD EKNKRASVRR
RIDQSNSHAN IFYSVLTIDK MQNKDKGLYT CRVRSGPSFK
SVNTSVHIYD KAFITVKHRK QQVLETVAGK RSYRLSMKVK
AFPSPEVVWL KDGLPATEKS ARYLTRGYSL IIKDVTEEDA
GNYTILLSIK QSNVFKNLTA TLIVNVKPQI YEKAVSSFPD
PALYPLGSRQ ILTCTAYGIP QPTIKWFWHP CNHNHSEARC
DFCSNNEESF ILDADSNMGN RIESITQRMA IIEGKNKMAS
TLVVADSRIS GIYICIASNK VGTVGRNISF YITDVPNGFH
VNLEKMPTEG EDLKLSCTVN KFLYRDVTWI LLRTVNNRTM
HYSISKQKMA ITKEHSITLN LTIMNVSLQD SGTYACRARN
VYTGEEILQK KEITIRDQEA PYLLRNLSDH TVAISSSTTL
DCHANGVPEP QITWFKNNHK IQQEPGIILG PGSSTLFIER
VTEEDEGVYH CKATNQKGSV ESSAYLTVQG TSDKSNLELI
TLTCTCVAAT LFWLLLTLFI RKMKRSSSEI KTDYLSIIMD
PDEVPLDEQC ERLPYDASKW EFARERLKLG KSLGRGAFGK
VVQASAFGIK KSPTCRTVAV KMLKEGATAS EYKALMTELK
ILTHIGHHLN VVNLLGACTK QGGPLMVIVE YCKYGNLSNY
LKSKRDLFFL NKDAALHMEP KKEKMEPGLE QGKKPRLDSV
TSSESFASSG FQEDKSLSDV EEEEDSDGFY KEPITMEDLI
SYSFQVARGM EFLSSRKGIH RDLAARNILL SENNVVKICD
FGLARDIYKN PDYVRKGDTR LPLKWMAPES IFDKIYSTKS
DVWSYGVLLW EIFSLGGSPY PGVQMDEDFC SRLREGMRMR
APEYSTPEIY QIMLDCWHRD PKERPRFAEL VEKLGDLLQA
NVQQDGKDYI PINAILTGNS GFTYSTPAFS EDFFKESISA
PKFNSGSSDD VRYVNAFKFM SLERIKTFEE LLPNATSMFD
DYQGDSSTLL ASPMLKRFTW TDSKPKASLK IDLRVTSKSK
ESGLSDVSRP SFCHSSCGHV SEGKRRFTYD HAELERKIAC
CSPPPDYNSV VLYSTPPI
human VEGFR2 sequence from Uniparc
entry UPI000003AE04,
SEQ ID NO: 2
MQSKVLLAVA LWLCVETRAA SVGLPSVSLD LPRLSIQKDI
LTIKANTTLQ ITCRGQRDLD WLWPNNQSGS EQRVEVTECS
DGLECKTLTI PKVIGNDTGA YKCFYRETDL ASVIYVYVQD
YRSPFIASVS DQHGVVYITE NKNKTVVIPC LGSISNLNVS
LCARYPEKRF VPDGNRISWD SKKGFTIPSY MISYAGMVEC
EAKINDESYQ SIMYIVVVVG YRIYDVVLSP SHGIELSVGE
KLVLNCTART ELNVGIDFNW EYPSSKHQHK KLVNRDLKTQ
SGSEMKKELS TLTIDGVTRS DQGLYTCAAS SGLMTKKNST
FVRVHEKPFV AFGSGMESLV EATVGERVRI PAKYLGYPPP
EIKWYKNGIP LESNHTIKAG HVLTIMEVSE RDTGNYTVIL
TNPISKEKQS HVVSLVVYVP PQIGEKSLIS PVDSYQYGTT
QTLTCTVYAI PPPHHIHWYW QLEEECANEP SQAVSVTNPY
PCEEWRSVED FQGGNKIEVN KNQFALIEGK NKTVSTLVIQ
AANVSALYKC EAVNKVGRGE RVISFHVTRG PEITLQPDMQ
PTEQESVSLW CTADRSTFEN LTWYKLGPQP LPIHVGELPT
PVCKNLDTLW KLNATMFSNS TNDILIMELK NASLQDQGDY
VCLAQDRKTK KRHCVVRQLT VLERVAPTIT GNLENQTTSI
GESIEVSCTA SGNPPPQIMW FKDNETLVED SGIVLKDGNR
NLTIRRVRKE DEGLYTCQAC SVLGCAKVEA FFIIEGAQEK
TNLEIIILVG TAVIAMFFWL LLVIILRTVK RANGGELKTG
YLSIVMDPDE LPLDEHCERL PYDASKWEFP RDRLKLGKPL
GRGAFGQVIE ADAFGIDKTA TCRTVAVKML KEGATHSEHR
ALMSELKILI HIGHHLNVVN LLGACTKPGG PLMVIVEFCK
FGNLSTYLRS KRNEFVPYKT KGARFRQGKD YVGAIPVDLK
RRLDSITSSQ SSASSGFVEE KSLSDVEEEE APEDLYKDFL
TLEHLICYSF QVAKGMEFLA SRKCIHRDLA ARNILLSEKN
VVKICDFGLA RDIYKDPDYV RKGDARLPLK WMAPETIFDR
VYTIQSDVWS FGVLLWEIFS LGASPYPGVK IDEEFCRRLK
EGTRMRAPDY TTPEMYQTML DCWHGEPSQR PTFSELVEHL
GNLLQANAQQ DGKDYIVLPI SETLSMEEDS GLSLPTSPVS
CMEEEEVCDP KFHYDNTAGI SQYLQNSKRK SRPVSVKTFE
DIPLEEPEVK VIPDDNQTDS GMVLASEELK TLEDRTKLSP
SFGGMVPSKS RESVASEGSN QTSGYQSGYH SDDTDTTVYS
SEEAELLKLI EIGVQTGSTA QILQPDSGTT LSSPPV
human VEGFR3 sequence from Uniparc
entry UPI00001488E7,
SEQ ID NO: 3
MQRGAALCLR LWLCLGLLDG LVSGYSMTPP TLNITEESHV
IDTGDSLSIS CRGQHPLEWA WPGAQEAPAT GDKDSEDTGV
VRDCEGTDAR PYCKVLLLHE VHANDTGSYV CYYKYIKARI
EGTTAASSYV FVRDFEQPFI NKPDTLLVNR KDAMWVPCLV
SIPGLNVTLR SQSSVLWPDG QEVVWDDRRG MLVSTPLLHD
ALYLQCETTW GDQDFLSNPF LVHITGNELY DIQLLPRKSL
ELLVGEKLVL NCTVWAEFNS GVTFDWDYPG KQAERGKWVP
ERRSQQTHTE LSSILTIHNV SQHDLGSYVC KANNGIQRFR
ESTEVIVHEN PFISVEWLKG PILEATAGDE LVKLPVKLAA
YPPPEFQWYK DGKALSGRHS PHALVLKEVT EASTGTYTLA
LWNSAAGLRR NISLELVVNV PPQIHEKEAS SPSIYSRHSR
QALTCTAYGV PLPLSIQWHW RPWTPCKMFA QRSLRRRQQQ
DLMPQCRDWR AVTTQDAVNP IESLDTWTEF VEGKNKTVSK
LVIQNANVSA MYKCVVSNKV GQDERLIYFY VTTIPDGFTI
ESKPSEELLE GQPVLLSCQA DSYKYEHLRW YRLNLSTLHD
AHGNPLLLDC KNVHLFATPL AASLEEVAPG ARHATLSLSI
PRVAPEHEGH YVCEVQDRRS HDKHCHKKYL SVQALEAPRL
TQNLTDLLVN VSDSLEMQCL VAGAHAPSIV WYKDERLLEE
KSGVDLADSN QKLSIQRVRE EDAGRYLCSV CNAKGCVNSS
ASVAVEGSED KGSMEIVILV GTGVIAVFFW VLLLLIFCNM
RRPAHADIKT GYLSIIMDPG EVPLEEQCEY LSYDASQWEF
PRERLHLGRV LGYGAFGKVV EASAFGIHKG SSCDTVAVKM
LKEGATASEH RALMSELKIL IHIGNHLNVV NLLGACTKPQ
GPLMVIVEFC KYGNLSNFLR AKRDAFSPCA EKSPEQRGRF
RAMVELARLD RRRPGSSDRV LFARFSKTEG GARRASPDQE
AEDLWLSPLT MEDLVCYSFQ VARGMEFLAS RKCIHRDLAA
RNILLSESDV VKICDFGLAR DIYKDPDYVR KGSARLPLKW
MAPESIFDKV YTTQSDVWSF GVLLWEIFSL GASPYPGVQI
NEEFCQRLRD GTRMRAPELA TPAIRRIMLN CWSGDPKARP
AFSELVEILG DLLQGRGLQE EEEVCMAPRS SQSSEEGSFS
QVSTMALHIA QADAEDSPPS LQRHSLAARY YNWVSFPGCL
ARGAETRGSS RMKTFEEFPM TPTTYKGSVD NQTDSGMVLA
SEEFEQIESR HRQESGFSCK GPGQNVAVTR AHPDSQGRRR
RPERGARGGQ VFYNSEYGEL SEPSEEDHCS PSARVTFFTD
NSY
human VEGF-A sequence from Uniparc
entry UPI0000030866,
SEQ ID NO: 4
MNFLLSWVHW SLALLLYLHH AKWSQAAPMA EGGGQNHHEV
VKFMDVYQRS YCHPIETLVD IFQEYPDEIE YIFKPSCVPL
MRCGGCCNDE GLECVPTEES NITMQIMRIK PHQGQHIGEM
SFLQHNKCEC RPKKDRARQE KKSVRGKGKG QKRKRKKSRY
KSWSVYVGAR CCLMPWSLPG PHPCGPCSER RKHLFVQDPQ
TCKCSCKNTD SRCKARQLEL NERTCRCDKP RR
human VEGF-B sequence from Uniparc
entry UPI0000001047,
SEQ ID NO: 5
MSPLLRRLLL AALLQLAPAQ APVSQPDAPG HQRKVVSWID
VYTRATCQPR EVVVPLTVEL MGTVAKQLVP SCVTVQRCGG
CCPDDGLECV PTGQHQVRMQ ILMIRYPSSQ LGEMSLEEHS
QCECRPKKKD SAVKPDRAAT PHHRPQPRSV PGWDSAPGAP
SPADITHPTP APGPSAHAAP STTSALTPGP AAAAADAAAS
SVAKGGA,
human VEGF-C sequence from Uniparc
entry UPI0000001C2A,
SEQ ID NO: 6
MHLLGFFSVA CSLLAAALLP GPREAPAAAA AFESGLDLSD
AEPDAGEATA YASKDLEEQL RSVSSVDELM TVLYPEYWKM
YKCQLRKGGW QHNREQANLN SRTEETIKFA AAHYNTEILK
SIDNEWRKTQ CMPREVCIDV GKEFGVATNT FFKPPCVSVY
RCGGCCNSEG LQCMNTSTSY LSKTLFEITV PLSQGPKPVT
ISFANHTSCR CMSKLDVYRQ VHSIIRRSLP ATLPQCQAAN
KTCPTNYMWN NHICRCLAQE DFMFSSDAGD DSTDGFHDIC
GPNKELDEET CQCVCRAGLR PASCGPHKEL DRNSCQCVCK
NKLFPSQCGA NREFDENTCQ CVCKRTCPRN QPLNPGKCAC
ECTESPQKCL LKGKKFHHQT CSCYRRPCTN RQKACEPGFS
YSEEVCRCVP SYWKRPQMS
human VEGF-D sequence from Uniparc
entry UPI00000012B2,
SEQ ID NO: 7
MYREWVVVNV FMMLYVQLVQ GSSNEHGPVK RSSQSTLERS
EQQIRAASSL EELLRITHSE DWKLWRCRLR LKSFTSMDSR
SASHRSTRFA ATFYDIETLK VIDEEWQRTQ CSPRETCVEV
ASELGKSTNT FFKPPCVNVF RCGGCCNEES LICMNTSTSY
ISKQLFEISV PLTSVPELVP VKVANHTGCK CLPTAPRHPY
SIIRRSIQIP EEDRCSHSKK LCPIDMLWDS NKCKCVLQEE
NPLAGTEDHS HLQEPALCGP HMMFDEDRCE CVCKTPCPKD
LIQHPKNCSC FECKESLETC CQKHKLFHPD TCSCEDRCPF
HTRPCASGKT ACAKHCREPK EKRAAQGPHS RKNP,
human P1GF precursor sequence from
Uniparc entry UPI000013IBEF,
SEQ ID NO: 8
MPVMRLFPCF LQLLAGLALP AVPPQQWALS AGNGSSEVEV
VPFQEVWGRS YCRALERLVD VVSEYPSEVE HMFSPSCVSL
LRCTGCCGDE NLHCVPVETA NVTMQLLKIR SGDRPSYVEL
TFSQHVRCEC RHSPGRQSPD MPGDFRADAP SFLPPRRSLP
MLERMEWGGA LTGSQSAVWP SSPVPEEIPR MHPGRNGKKQ
QRKPLREKMK PERCGDAVPR R

### B. Polymer Compositions

Also provided herein are polymer compositions. Typically, a polymer composition as described herein includes a functional polymer, a crosslinker, and water. In some embodiments, the polymer compositions described herein are injectable into the middle and/or inner ear, where the functional polymers crosslink ionically and/or covalently, to generate a crosslinked polymer composition in the form of a gel (e.g., hydrogel). Crosslinking can occur upon mixing and injecting the polymer composition, by altering PHK exposure to ions, and/or exposure to a photocrosslinker.

In some embodiments, a polymer composition as described herein can have one or more functional properties that are advantageous for administration to a subject (e.g., to the middle and/or inner ear of a subject).

In some cases, a polymer composition can be characterized by the ability to crosslink and to form a durable gel (e.g., hydrogel), for example, in situ. The phenomenon of transition from a solution to a gel is commonly referred to as sol-gel transition. The sol-gel transition of a polymer composition can be experimentally verified by a number of techniques such as the vial inversion method, spectroscopy, differential scanning calorimetry (DSC), and rheology.

In some embodiments, a gel formed from a polymer composition described herein can have a gel duration of at least 20 days (e.g., at least 30 days, at least 40 days, at least 50 days, at least 60 days, at least 70 days, at least 80 days, at least 90 days, at least 100 days, or more) when stored in an inverted vial at room temperature (e.g., 20° C.). In some embodiments, a gel formed from a polymer composition as described herein can have a gel duration of at least 5 days (e.g., at least 7 days, at least 10 days, at least 14 days, at least 18 days, at least 21 days, at least 25 days, at least 28 days, or more) when stored at body temperature (e.g., 37° C.), as measured by placing a gel (e.g., 200 μL) in a receptor solution (e.g., pH 7.4 PBS). In situ, the degradation of a gel (e.g., a hydrogel) formed from a polymer composition as described herein can depend on the identity of the components (e.g., functional polymer and/or crosslinker) of the polymer composition, as well as administration accuracy and subject metabolism. In some embodiments, a gel formed from a polymer composition as described herein can have a residence time of at least 5 days (e.g., at least 7 days, at least 10 days, at least 14 days, at least 18 days, at least 21 days, at least 25 days, at least 28 days, at least 42 days, at least 56 days, or more) when administered to the middle ear of a subject.

In some cases, a polymer composition can be characterized by the ability to crosslink and to form a shape conforming gel (e.g., hydrogel), for example, in situ. In some embodiments, a gel formed from a polymer composition (e.g., 200 μL of gel) described herein can retain its shape for at least 20 days (e.g., at least 30 days, at least 40 days, at least 50 days, at least 60 days, at least 70 days, at least 80 days, at least 90 days, at least 100 days, or more) when stored in an upright vial at 37° C. with 1 mL of PBS; inversion of the vial can be used to determine whether the polymer composition is still a gel.

In some embodiments, a polymer composition as described herein can form a gel that is significantly elastic, rather than significantly viscoelastic. Without being bound by any particular theory, it is believed that an elastic gel will have a greater residence time in a location of administration than a viscoelastic gel, which may flow away. For example, in some embodiments, a gel formed from a polymer composition described herein can have an elastic modulus of about 0.01 to about 100 kPa (e.g., about 0.1 to about 100 kPa, about 1 to about 100 kPa, about 5 to about 100 kPa, about 10 to about 100 kPa, about 25 to about 100 kPa, about 50 to about 100 kPa, about 75 to about 100 kPa, about 0.01 to about 0.1 kPa, about 0.01 to about 1 kPa, about 0.01 to about 5 kPa, about 0.01 to about 10 kPa, about 0.01 to about 25 kPa, about 0.01 to about 50 kPa, or about 0.01 to about 75 kPa).

In some embodiments, a polymer composition can have a wide transition in viscosity, e.g., some polymer compositions as described herein can flow (e.g., as a solution or suspension) to a target site and form a gel (e.g., hydrogel), for example, in situ. In some embodiments, a polymer composition can be injected in the form of a solution or suspension, flows by gravity throughout the middle ear or to one or more sites in the middle ear, and then forms a gel (e.g., hydrogel). In some such embodiments, the polymer composition wets (e.g., completely wets) the round window membrane. In some embodiments, delivery of the polymer composition can be accomplished without bubbles, for example, bubbles in the gel and/or bubbles trapped between the gel and round window membrane. In some embodiments, the viscosity of the polymer composition (e.g., immediately after combining the functional polymer, crosslinker, and water), is between about 1 and about 100 mPa*s (e.g., about 2 and about 100 mPa*s, about 5 and about 100 mPa*s, about 10 and about 100 mPa*s, about 25 and about 100 mPa*s, about 50 and about 100 mPa*s, about 75 and about 100 mPa*s, about 1 and about 2 mPa*s, about 1 and about 5 mPa*s, about 1 and about 10 mPa*s, about 1 and about 25 mPa*s, about 1 and about 50 mPa*s, or about 1 and about 75 mPa*s). In some cases, a polymer composition as described herein can be injected using a 23-gauge (23 G) needle, or a needle of high gauge (smaller diameter), e.g., without significant clogging of the needle.

In some cases, a polymer composition can be characterized by the ability to crosslink and to form a gel (e.g., hydrogel), for example, in situ, with a gelation time that is suitable for administration to a site (e.g., the middle and/or inner ear) of a subject. Some polymer compositions, such as DURASEAL®, can form gels in 3 seconds or less, which may not be desirable for delivery of an extended release otic composition as disclosed herein.

In some embodiments, the polymer compositions described herein can have a gelation time of about 45 seconds to about 60 min (e.g., about 1 minute to about 60 min, about 45 seconds to about 45 min, about 45 seconds to about 30 min, about 45 seconds to about 20 min, about 45 seconds to about 10 min, about 45 seconds to about 8 min, about 45 seconds to about 5 min, about 45 seconds to about 3 min, about 45 seconds to about 2 min about 45 seconds to about 1 minute, about 1 minute to about 60 min, about 2 min to about 60 min, about 3 min to about 60 min, about 4 min to about 60 min, about 5 min to about 60 min, about 8 min to about 60 min, about 10 min to about 60 min, about 20 min to about 60 min, about 30 min to about 60 min, about 45 min to about 60 min, about 1 minute to about 5 min, about 5 min to about 20 min, about 8 min to about 12 min, about 4 min to about 12 min, or about 1 min to about 8 min) at a temperature of about 20° C. In some embodiments, the polymer compositions described herein can have a gelation time of about 8 minutes to about 16 minutes (e.g., about 9 minutes to about 15 minutes, about 10 minutes to about 14 minutes, or about 11 minutes to about 13 minutes) at a temperature of about 20° C. In some embodiments, the polymer compositions described herein can have a gelation time of about 9 minutes to about 11 minutes at a temperature of about 20° C. In some embodiments, the polymer compositions described herein can have a gelation time of at least about 45 seconds (e.g., at least about 1 minute, at least about 2 min, at least about 3 min, at least about 5 min, or at least about 10 min) at a temperature of about 20° C. In some embodiments, the polymer compositions described herein can have a gelation time of about 8 min, 9 min, 10 min, 11 min, or 12 min at a temperature of about 20° C.

In some embodiments, the polymer compositions described herein can have a gelation time of about 10 s to about 30 min (e.g., about 10 s to about 30 s, about 10 s to about 1 min, about 10 s to about 2 min, about 10 s to about 3 min, about 10 s to about 4 min, about 10 s to about 5 min, about 10 s to about 8 min, about 30 s to about 10 min, about 30 s to about 15 min, about 30 s to about 20 min, about 1 min to about 30 min, about 2 min to about 30 min, about 3 min to about 30 min, about 4 min to about 30 min, about 5 min to about 30 min, about 8 min to about 30 min, about 10 min to about 30 min, about 15 min to about 30 min, about 20 min to about 30 min, about 30 s to about 3 min, about 1 min to about 2 min, about 2 min to about 8 min, about 3 min to about 8 min, about 2 min to about 6 min, about 4 min to about 6 min, about 3 min to about 5 min, or about 1 min to about 4 min) at about 37° C. In some embodiments, the polymer compositions described herein can have a gelation time of about 30 s to about 2 min (e.g., about 45 s to about 1 min) at about 37° C. In some embodiments, the polymer compositions described herein can have a gelation time of at least about 10 s (e.g., at least about 30 s, at least about 1 min, at least about 2 min, at least about 3 min, at least about 4 min, at least about 5 min, at least about 8 min, or at least about 10 min), and optionally less than 30 minutes (e.g., less than 15 minutes) at about 37° C. In some embodiments, the polymer compositions described herein can have a gelation time of about 3 min, 4 min, 5 min, 6 min, 7 min, or 8 min at a temperature of about 37° C.

Without being bound by any particular theory, it is believed that the gelation time of a polymer composition or extended release otic composition can impact its usefulness in a clinical setting, as compositions that gel too quickly may decrease the amount of time in which a composition can be administered to the ear of a subject (e.g., via transtympanic injection). In some embodiments, a polymer composition or extended release otic composition provided here is injectable through a 27 gauge (or larger diameter) needle for at least 10 minutes after the functional polymer and crosslinker are combined. In some embodiments, a polymer composition or extended release otic composition provided here is injectable through a 27 gauge (or larger diameter) needle for at least 2 minutes (e.g., 2 to 4 minutes, 2 to 6 minutes, 2 to 8 minutes, 2 to 10 minutes) after the functional polymer and crosslinker are combined.

The rate of a crosslinking reaction can be influenced by selection of properties of the polymer composition; for example, pH, amounts of crosslinker and functionalized polymer, and buffer selection can affect gelation time. Mixing components while chilled can also slow down the crosslinking reaction to allow time for injection. In some cases, a polymer composition or an extended release otic composition can adhere to tissue in the middle ear and to create a sufficiently crosslinked gel that will be maintained for long durations in the middle ear before degrading into liquid.

Controlled rates of active agent delivery also may be obtained with the system by degradable, covalent attachment of the molecules to the crosslinked hydrogel network. The nature of the covalent attachment can be controlled to enable control of the release rate from hours to weeks or longer. By using a composite made from linkages with a range of hydrolysis times, a controlled release profile may be extended for longer durations.

In some cases, a gel formed from a polymer composition or extended release otic composition as described herein can exhibit a swelling of less than about 150% (e.g., less than about 140%, less than about 120%, less than about 100%, less than about 80%, less than about 70%, less than about 60%, less than about 50%, or less than about 40%), for example, when incubated in an excess of phosphate buffered saline (PBS) at 37° C. (e.g., for about 1 day, or for about 2 days). In some cases, a gel formed from a polymer composition or extended release otic composition as described herein can exhibit a swelling of about 25% to about 45% (e.g., about 30% to about 40%), for example, when incubated in an excess of phosphate buffered saline (PBS) at 37° C. after 1 day. In some cases, a gel formed from a polymer composition or extended release otic composition as described herein can exhibit a swelling of about 40% to about 80% (e.g., about 50% to about 70%), for example, when incubated in an excess of phosphate buffered saline (PBS) at 37° C. after 7 days. In some embodiments, a gel formed from a polymer composition or extended release otic composition can have a resorption time (e.g., time to where the mass of the gel incubated in an excess of PBS at 50° C. is less than the starting mass of the gel) of about 5 days to about 30 days (e.g., about 5 days to about 10 days, about 5 days to about 15 days, about 5 days to about 20 days, about 5 days to about 25 days, about 10 days to about 30 days, about 15 days to about 30 days, about 20 days to about 30 days, about 25 days to about 30 days, about 7 days to about 9 days, or about 7 days to about 15 days). In some embodiments, a gel formed from a polymer composition or extended release otic composition described herein can be characterized by demonstrating adhesion to a surface in contact with polymer composition during gelation. In some embodiments, adhesion can be mechanical adhesion, gel adhesion, chemical adhesion, mucoadhesion, or a combination thereof. In some cases, a polymer composition can demonstrate adhesion to a tissue within the ear (e.g., the round window membrane). This can be achieved, for example, by crosslinking of one or more components of the polymer formulation, which may include a component that forms covalent crosslinks with tissue. As an example, a polymer composition can include amine-reactive and/or thiol-reactive groups, which can bind to tissue. In some embodiments, a gel formed from a polymer composition described herein, can be characterized by demonstrating mucoadhesion to a mucosal surface (e.g., the round window membrane) in contact with the polymer composition during gelation. In some embodiments, adhesion can be achieved though the formation of covalent bonds between the amine groups of proteins in tissue and activated esters of functional polymer.

An active agent (e.g., a therapeutic, prophylactic and/or visualization or diagnostic agent), for example, any of one or more the active agents described herein, can be included in any of the polymer compositions described herein. Typically, when an active agent is present in a polymer composition described herein, such a composition is termed an extended-release otic composition.

An active agent can be present in a polymer composition as described herein in any appropriate amount or concentration. In some embodiments, an active agent can be present in a polymer composition in an amount sufficient to deliver a therapeutically effective concentration to a site of action (e.g., through the round window membrane) for a period of time. In some embodiments, a period of time can be less than or equal to the residence time of the extended release otic composition in the site of administration. For example, in some embodiments, a period of time can be about 5 days to about 6 months (e.g., about 5 days to about 1 week, about 5 days to about 2 weeks, about 5 days to about 3 weeks, about 5 days to about 1 month, about 5 days to about 2 months, about 5 days to about 3 months, about 5 days to about 4 months, about 5 days to about 5 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, about 2 weeks to about 2 months, or about 1 month to about 3 months). In some embodiments, an active agent can be present in an amount of about 0.01% to about 40% (e.g., about 0.01% to about 0.1%, about 0.01% to about 1%, about 0.01% to about 2%, about 0.01% to about 3%, about 0.01% to about 5%, about 0.01% to about 8%, about 0.01% to about 10%, about 0.01% to about 12%, about 0.01% to about 15%, about 0.01% to about 20%, about 0.01% to about 20%, about 0.01% to about 40%, about 0.1% to about 40%, about 1% to about 40%, about 2% to about 40%, about 3% to about 40%, about 5% to about 40%, about 8% to about 40%, about 10% to about 40%, or about 12% to about 40%) by weight of the polymer composition. In some embodiments, an active agent can be present in an amount of about 1% to about 10% (e.g., about 2% to about 9%, about 3% to about 8%, or about 4% to about 6%) by weight of the polymer composition. In some embodiments, an active agent can be present in an amount of about 4%, 5%, 6%, 7%, or 8% by weight of the polymer composition. In some embodiments, an active agent can be present in an amount of about 8% to about 18% (e.g., about 8% to about 10%, about 8% to about 12%, about 8% to about 14%, about 8% to about 16%, about 10% to about 18%, about 12% to about 18%, about 14% to about 18%, about 16% to about 18%, about 9% to about 16%, or about 10% to about 15%) by weight of the polymer composition. In some embodiments, an active agent can be present in an amount of about 10%, 11%, 12%, 13%, 14%, or 15% by weight of the polymer composition.

An active agent can be present in a polymer composition in any appropriate form. In some embodiments, an active agent can be present in a polymer composition as a solution or as a suspension. In some embodiments, an active agent can be present in a polymer composition in the form of microparticles or nanoparticles. In some embodiments, an active agent can change from solution to suspension from one form of microparticle or nanoparticle to another microparticle or nanoparticle after combining the components of the extended release otic composition.

A generally preferred characteristic of a drug suspension composition is the ability to administer uniform doses. This can be easier to achieve for compositions that do not form a dense sediment during storage and can be easily re-dispersed upon manual agitation of a container. In some embodiments, extended release otic compositions can contain particles of an active agent that are easy to disperse with manual agitation and have a low viscosity such that they are easy to inject. This can be achieved, in some cases, by addition of a flocculating agent that promotes aggregation of drug particles into loose floccules. The flocculation efficiency can be defined as the ratio of final sediment volume (e.g., as a percentage of the total volume) to particle concentration. The final sediment volume cannot be greater than 100%, thereby limiting upper values of flocculation efficiency. In some embodiments, extended release otic compositions as described herein have a flocculation efficiency greater than about 3 (e.g., greater than about 4, or greater than about 5). In some embodiments, the choice of functional polymer and/or crosslinking agent can affect the flocculation efficiency of a given active agent in solution. For example, in some embodiments, the use of a polylysine (e.g., trilysine or tetralysine) or a salt thereof as crosslinker can provide a high degree of flocculation without the addition of other dispersing agents. It can be desirable for the crosslinking agent to also serve as a flocculating agent to minimize the number of excipients in an extended release formulation. Other amphiphilic crosslinkers, such as tetralysine, can also be used for dual purposes of crosslinking and flocculating drug particles.

In some embodiments, the active agent is stored as a dry powder until combined with other components of an extended release otic composition (e.g., at or near time of use). For example, microparticles and nanoparticles prepared by spray drying and/or super critical fluid processing can form loose aggregates that have good flow and handling properties and are easily dispersed to primary particles with low input of energy. When the components of an extended release otic composition are combined, the aggregates can be separated into primary particles by shear generated via introduction of air and/or other components of the extended release otic composition.

As noted above, polymer compositions as described herein typically include a functional polymer, a crosslinker, and water. Gels (e.g., hydrogels) can be formed from the reaction of functional polymers and crosslinkers having functional groups, e.g., electrophilic or nucleophilic functional groups.

As used herein, a “functional polymer” can be a polymer including one or more functional groups that can react with one or more functional groups on a crosslinker to form a bond (e.g., a covalent bond).

As used herein, a “crosslinker” can be a molecule including one or more functional groups that can react with one or more functional groups on a functional polymer to form a bond (e.g., a covalent bond). In some cases, a crosslinker is a polymer (e.g., trilysine or tetralysine, or a salt thereof). In some cases, a crosslinker is not a polymer.

In general, functional polymers and crosslinkers are water soluble, non-toxic and biologically acceptable. In some embodiments, a crosslinker is a small molecule. In some embodiments, a crosslinker has a solubility of at least 1 g/100 mL in an aqueous solution. In some embodiments, a functional polymer is a macromolecule. Exemplary classes of functional polymers and crosslinkers are described in U.S. Pat. Nos. 6,566,406; 6,887,974; 7,332,566; and 8,535,705, each of which is incorporated herein by reference in its entirety.

In some embodiments, a functional polymer or a crosslinker can be multifunctional, meaning that it comprises two or more functional groups. In some embodiments, a multifunctional functional polymer or crosslinker has only one type of functional group (e.g., all nucleophilic or all electrophilic functional groups). In some cases, a functional polymer or a crosslinker can include at least three (e.g., at least four, at least five, or more) functional groups, so that, as a result of reactions (e.g., electrophilic-nucleophilic reactions), the functional polymer and the crosslinker combine to form a crosslinked gel (e.g., hydrogel). Such reactions are typically referred to as “crosslinking reactions”.

A functional polymer can include a plurality of a first functional group. A crosslinker can include a plurality of a second functional group. In some such embodiments, the first functional group of the polymer can form covalent linkages with the second functional group of the crosslinker, thereby generating a gel (e.g., hydrogel). The distribution of functional groups can be any appropriate distribution. In some embodiments, the functional polymer is a branched polymer, where the terminus of each branch is functionalized with the first functional group. In some embodiments, the functional polymer can include a first type of monomer, where the functional polymer is a homopolymer of the first type of monomer. In some embodiments, the functional polymer can include a first type of monomer, where each of the first type of monomer includes the first functional group. In some cases, the first type of monomer can be randomly distributed in the functional polymer. In some cases, the first type of monomer can be regularly distributed (e.g., as block co-polymer or as an alternating co-polymer) in the functional polymer. In some cases, the first type of monomer can be part of a grafted co-polymer (e.g., as the backbone or as a branch) in the functional polymer. A functional polymer can be of any appropriate size. In some embodiments, a functional polymer is a macromolecule, for example, a macromolecule with a Mₙ of 5,000, 10,000, 20,000, 30,000, or more.

In some embodiments, the crosslinker can include a second type of monomer, where the functional polymer is a homopolymer of the second type of monomer. In some embodiments, the crosslinker is a branched polymer, where the terminus of each branch is functionalized with the second functional group. In some embodiments, the crosslinker can include a second type of monomer, where each of the second type of monomer includes the second functional group. In some cases, the second type of monomer can be randomly distributed in the crosslinker. In some cases, the second type of monomer can be regularly distributed (e.g., as block co-polymer or as an alternating co-polymer) in the crosslinker. In some cases, the second type of monomer can be part of a grafted co-polymer (e.g., as the backbone or as a branch) in the crosslinker. A crosslinker can be any appropriate size. In some embodiments, a crosslinker is a small molecule. In some embodiments, a crosslinker is an oligomer, for example, a dimer, a trimer, a tetramer, or a pentamer.

It will be appreciated that a first functional group (e.g., on a functional polymer) and a second functional group (e.g., on a crosslinker) should be such that a crosslinking reaction can occur. Therefore, the choice of functional polymer can be based on the choice of crosslinker, or vice versa. In some embodiments, a first functional group can be a NHS group and a second functional group can be an amine (e.g., a primary amine), or vice versa.

In some cases, the functional polymer contains only electrophilic or nucleophilic functional groups, and the crosslinker contains only nucleophilic or electrophilic functional groups, respectively. Thus, for example, if a crosslinker has nucleophilic functional groups such as amines (e.g., primary amines), the functional polymer, in some cases, may only have electrophilic functional groups such as N-hydroxysuccinimides. If, for example, a crosslinker has electrophilic functional groups such as sulfosuccinimides, then, in some cases the functional polymer may have nucleophilic functional groups such as amines (e.g., primary amines).

A functional polymer can be present in any appropriate concentration in a polymer composition as described herein. In some embodiments, a functional polymer can be present in a concentration of about 5% to about 15% (e.g., about 5% to about 7%, about 5% to about 9%, about 5% to about 11%, about 5% to about 13%, about 7% to about 15%, about 9% to about 15%, about 11% to about 15%, about 13% to about 15%, about 7% to about 13%, about 8% to about 11%, about 6% to about 12%, or about 7% to about 10%) by weight of the polymer composition. In some embodiments, a functional polymer can be present in a concentration of about 6%, about 7%, about 8%, about 9%, about 10%, or about 11% by weight of the polymer composition. In some embodiments, a functional polymer can be present in a concentration of about 8.3% by weight of the polymer composition. In some embodiments, a functional polymer can be present in an extended release otic composition as in a polymer composition as described herein.

A crosslinker can be present in any appropriate concentration in a polymer composition as described herein. In some embodiments, a crosslinker can be present in a concentration of about 0.2% to about 0.6% (e.g., about 0.2% to about 0.4%, about 0.4% to about 0.6%, or about 0.3% to about 0.5%) by weight of the polymer composition. In some embodiments, a crosslinker can be present in a concentration of about 0.05% to about 0.6% (e.g., about 0.05% to about 0.2%, about 0.05% to about 0.4%, about 0.05% to about 0.5%, about 0.1% to about 0.6%, about 0.2% to about 0.6%, about 0.4% to about 0.6%, or about 0.1% to about 0.3%) by weight of the polymer composition. In some embodiments, a crosslinker can be present in a concentration of about 0.1%, 0.2%, 0.3%, 0.4%, 0.5%, or 0.6% by weight of the polymer composition. In some embodiments, a crosslinker can be present in a concentration of about 0.05% to about 10% (e.g., about 0.05% to about 0.5%, about 0.05% to about 1%, about 0.05% to about 2%, about 0.05% to about 3%, about 0.05% to about 5%, about 0.05% to about 7%, about 0.05% to about 9%, about 0.5% to about 10%, about 1% to about 10%, about 2% to about 10%, about 3% to about 10%, about 5% to about 10%, about 7% to about 10%, about 9% to about 10%, or about 0.5% to about 2%) by weight of the polymer composition. In some embodiments, a crosslinker can be present in an extended release otic composition as in a polymer composition as described herein.

In some embodiments, a crosslinker can include amine (e.g., primary amine) groups, or salts (e.g., acetate salts) thereof. In some embodiments, a crosslinker can be a polylysine, for example a trilysine, or a salt (e.g., acetate salt) thereof. It will be understood that the weight percentage of a crosslinker, when in a salt form, will correspond to a smaller weight percentage of the crosslinker when not in salt form (e.g., as the free base), as the salt provides additional molar mass. As an illustrative example, if the crosslinker is trilysine acetate, then the weight percentage of the crosslinker in the polymer composition or extended release otic composition for trilysine free base is approximately 69% of the weight percentage of trilysine acetate, all else being the same.

The functional groups can be present in any appropriate ratio. In some embodiments, where a first functional group and a second functional group are used (e.g., an electrophilic functional group a nucleophilic functional group), the ratio of the first functional group to the second functional group can be between about 1.2 equivalents first functional group: 0.8 equivalents second functional group to about 0.8 equivalents first functional group: 1.2 equivalents second functional group. In some embodiments, the ratio of the first functional group to the second functional group can be between about 1 equivalent first functional group: 0.9 equivalents second functional group to about 0.9 equivalents first functional group: 1 equivalent second functional group. In some embodiments, the ratio of the first functional group to the second functional group is about 1:1. It will be understood that the molar ratio or weight ratio of the functional polymer and the crosslinker will be based on the relative number of functional groups per functional polymer or crosslinker, respectively.

In some embodiments, a functional polymer can be a modified polyethylene glycol (PEG) polymer. Exemplary modified PEG polymers include linear, branched, or multi-arm water soluble polymers including a plurality of polyethylene glycol units and (e.g., as part of a monomer or as an end-cap) two or more instances of functional groups (e.g., a succinimidyl ester (e.g., N-hydroxysuccinimide ester (NHS)), a sulfo-succinimidyl ester, epoxide or similar reactive groups). A multi-arm functional polymer can include a water soluble core, for example, sugars (xylitol, erythritol), glycerol, or trimethylolpropane. A water soluble core can be extended, optionally with at least one biodegradable linkage between it and each terminal group, which in some cases can be a functional group. A biodegradable linkage can, in some cases, be a single linkage or copolymers or homopolymers of absorbable polymers such as polyhydroxy acids or polylactones.

In some embodiments, a functional polymer can include an enzymatically and/or hydrolytically cleavable link. For example, molecules cleaved by enzymes such as collagenase may be synthesized and inserted into the polymers using methods known to those skilled in the peptide synthesis art. In some embodiments, carboxyl-, amine- or hydroxy-terminated polyethylene glycol can be used as a starting material for building a suitable peptide sequence for enzymatic cleavage, and a terminal end of the peptide sequence is converted into a carboxylic acid by reacting succinic anhydride with an appropriate amino acid. The acid group generated can then be converted to an NHS ester by reaction with N-hydroxysuccinimide.

In some cases, a functional polymer can be purchased or prepared using a variety of synthetic methods.

A functional group on a functional polymer or a crosslinker can, in some embodiments, be a reactive functional group that is also water solubilizing, such an succinimidyl ester group further functionalized with a PEG or sulfonate group. An ionic group, like a metal salt (e.g., a sodium salt) of sulfonic acid, or a nonionic group, like a polyethylene oxide on the succinimide ring, can improve water solubility while the NHS ester provides chemical reactivity towards amines.

Functional polymers, such as polyethylene glycols, functionalized with reactive functional groups such as succinimidyl ester groups are commercially available from, for example, MilliporeSigma (Milwaukee, Wis.) and Creative PEGWorks (Chapel Hill, NC). Functional polymers, such as polyethylene glycols, functionalized with reactive functional groups such as primary amines and thiols are commercially available from, for example MilliporeSigma (Milwaukee, Wis.) and JenKem (Plano, Texas)). In some embodiments, commercially available polymers with terminal hydroxyl groups can be converted into functional polymers with amine groups by methods known in the art. Similarly, crosslinkers complementary to a functional polymer are typically commercially available from companies such as MilliporeSigma.

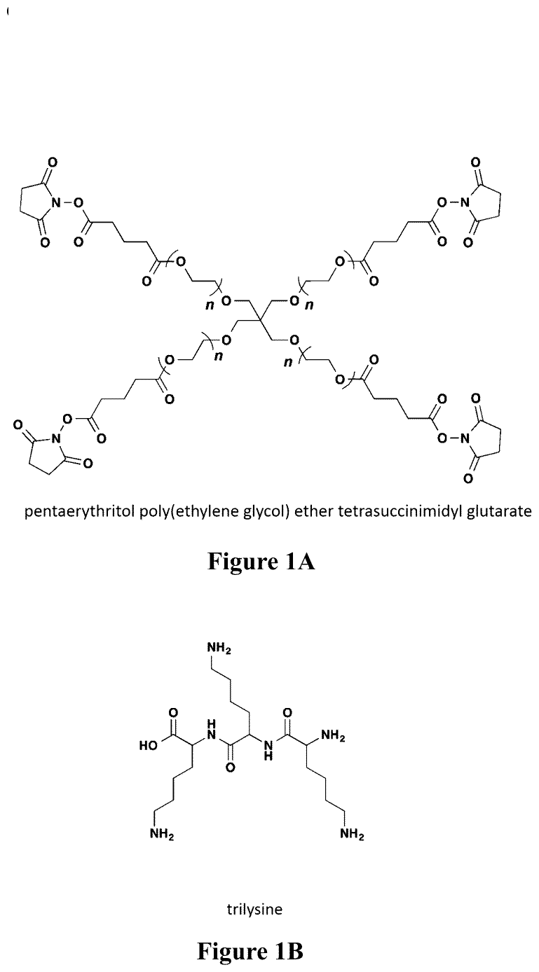

In some embodiments, the functional polymer is a multi-arm (e.g., 3-arm, 4-arm, 6-arm, or 8-arm) polyethylene glycol (PEG) including a plurality of (e.g., two more) succinimidyl functional groups (e.g., a succinimidyl succinate, a succinimidyl glutarate, a succinimidyl adipate, succinimidyl gluraramide, succinimidyl carbonate, or succinimidyl carboxymethyl ester) or sulfo-succinimidyl ester functional groups and the crosslinker contains a plurality of amine (e.g., primary amine) functional groups. In some embodiments, the functional polymer is a 4-arm PEG with a pentaerythritol core. In some embodiments, the functional polymer is an 8-arm PEG with a hexaglycerol core. In some embodiments, the functional polymer is an 8-arm PEG with a tripentaerythritol core. In some embodiments, the multi-arm PEG can have two or more arms terminate in a succinimidyl functional group. In some embodiments, one or more monomers of the multi-arm PEG can include a succinimidyl functional group. In some embodiments, the crosslinker can be a polylysine (e.g., an epsilon-polylysine) (e.g., trilysine, tetralysine, or pentalysine), or a salt (e.g., an acetate salt) thereof. For example, in some embodiments, the functional polymer can be pentaerythritol poly(ethylene glycol) ether tetrasuccinimidyl glutarate, and the crosslinker can be trilysine, or a salt thereof (shown in FIGS. 1A and 1B, respectively). In some embodiments, the functional polymer (e.g., pentaerythritol poly(ethylene glycol) ether tetrasuccinimidyl glutarate) can have a molecular weight (M_{N}) of about 10 kDa to about 25 kDa (e.g, about 10 kDa to about 15 kDa, about 10 kDa to about 20 kDa, about 15 kDa to about 25 kDa, or about 20 kDa to about 25 kDa). In some embodiments, the functional polymer (e.g., pentaerythritol poly(ethylene glycol) ether tetrasuccinimidyl glutarate) can have a molecular weight (Mw) of about 10 kDa to about 25 kDa (e.g, about 10 kDa to about 15 kDa, about 10 kDa to about 20 kDa, about 15 kDa to about 25 kDa, or about 20 kDa to about 25 kDa). Molecular weight can be determined, for example, by gas phase chromatography or matrix assisted laser desorption chromatography. In some embodiments, the functional polymer (e.g., pentaerythritol poly(ethylene glycol) ether tetrasuccinimidyl glutarate) can have a polydispersity (PD) of 1.5 f 0.5.

In some embodiments, the functional polymer is a multi-arm (e.g., 3-arm 4-arm, 6-arm, or 8-arm) polyethylene glycol including two or more amine (e.g., primary amine) functional groups and the crosslinker includes a plurality of succinimidyl ester (e.g., a succinimidyl succinate or succinimidyl glutarate) or sulfo-succinimidyl ester functional groups. In some embodiments, the multi-arm PEG can have two or more arms terminate in an amine (e.g., primary amine) functional group. In some embodiments, one or more monomers of the multi-arm PEG can include an amine (e.g., primary amine) functional group. In some embodiments, the crosslinker can be disuccinimidyl glutarate, disuccinimidyl suberate, bis(sulfosuccinimidyl)suberate, or disuccinimidyl succinate.

### Crosslinking Reactions

Typically, crosslinking reactions can occur under physiological conditions. In some embodiments, crosslinking reactions can occur “in situ”, meaning they occur at local sites such as on organs or tissues in a living animal or human body. In some embodiments, the crosslinking reactions do not release heat of polymerization.

Crosslinking between the functional polymer and the crosslinker can be initiated under any appropriate conditions. In some embodiments, the crosslinking between the functional polymer and the crosslinker is initiated upon the addition of a catalyst (e.g., an initiator molecule). In some embodiments, the crosslinking is initiated by a stimulus, e.g., a change in pH, temperature, or irradiation (e.g., using photo initiation). The rate of crosslinking and gelation time can be influenced by factors such as pH, temperature, excipients, ratio of functional groups, degree of functionalization of the functional polymer, and concentration of the functional polymer and crosslinker.

In some embodiments, the crosslinking between the functional polymer and the crosslinker is initiated upon mixing the functional polymer and the crosslinker. In some such embodiments, the gelation time is sufficient to allow administration of the polymer composition or extended release otic composition to a site of administration (e.g., an area of the middle and/or inner ear) in a significantly fluid form (e.g., by injection through a 23 G needle). In some embodiments, crosslinking can be initiated prior to or at the time of administration, but the polymer composition does not significantly gel prior to flowing into the site of administration (e.g., an area of the middle and/or inner ear). Viscosity typically increases with crosslinking, and it is therefore generally desirable to administer a polymer composition or an extended release otic composition before significant crosslinking has occurred. Accordingly, in some embodiments, a polymer composition or an extended release otic composition can have a viscosity of less than about 1000 mPa·s (e.g., less than about 800 mPa·s, 500 mPa·s, 300 mPa·s, 100 mPa·s, 75 mPa·s, 50 mPa·s, or 25 mPa·s) at a temperature of about 20° C. In some embodiments, a polymer composition or an extended release otic composition, can have a viscosity of about 1 mPa·s to about 100 mPa·s (e.g., about 1 mPa·s to about 80 mPa·s, about 1 mPa·s to about 60 mPa·s, about 1 mPa·s to about 50 mPa·s, about 1 mPa·s to about 40 mPa·s, about 1 mPa·s to about 20 mPa·s, about 1 mPa·s to about 10 mPa·s, about 10 mPa·s to about 100 mPa·s, about 20 to about 100, about 40 to about 100, about 50 to about 100 mPa·s, about 60 mPa·s to about 100 mPa·s, or about 80 mPa·s to about 100 mPa·s) at a temperature of about 20° C.

In some embodiments, pH is used to influence the gelation time. A reference product, DURASEAL®, marketed as a spine sealant, includes pentaerythritol poly(ethylene glycol) ether tetrasuccinimidyl glutarate and trilysine. DURASEAL®, prepared by the manufacturer's instructions, has a pH of about 10, and crosslinking typically occurs rapidly, and this product can form a gel in 3 seconds or less (see, e.g., Example 1). As described herein, adjusting the trilysine component to a pH of about 5.5 to about 8.5 (e.g., about 5.5 to 8.0, about 5.5 to about 7.5, about 5.5 to about 7.0, about 5.5 to about 6.5, about 5.5 to about 6.0, about 6.5 to about 7.0, about 6.5 to about 7.5, about 6.5 to about 8.0, about 7.0 to about 8.5, about 7.5 to about 8.5, or about 8.0 to about 8.5) can result in longer gelation times, such as those described hereinabove. In some embodiments, a polymer composition or an extended release otic composition can have a pH of about 5.5 to about 8.5 (e.g., about 5.5 to about 8.0, about 5.5 to about 7.5, about 5.5 to about 7.0, about 5.5 to about 6.5, about 5.5 to about 6.0, about 6.5 to about 7.0, about 6.5 to about 7.5, about 6.5 to about 8.0, about 7.0 to about 8.5, about 7.5 to about 8.5, or about 8.0 to about 8.5) In some embodiments, a polymer composition or an extended release otic composition can have a pH of about 5.0 to about 7.7 (e.g., about 5.0 to about 5.5, about 5.0 to about 6.0, about 5.0 to about 6.5, about 5.0 to about 7.0, about 5.0 to about 7.5, about 5.5 to about 7.7, about 6.0 to about 7.7, about 6.5 to about 7.7, about 7.0 to about 7.7, about 6.0 to about 7.0, about 6.6 to about 7.7, about 6.8 and about 7.7, about 6.6 to about 6.8). In some embodiments, a polymer composition or an extended release otic composition can have a pH of about 7.2. In some embodiments, a polymer composition or an extended release otic composition can have a pH of about 5.5 to about 6.5 (e.g., about 5.7 to about 6.2, or about 6.0). The pH of a polymer composition or an extended release otic composition can be adjusted by the addition of an acid (e.g., HCl, phosphoric acid), abase (e.g., NaOH, KOH), and/or a buffer (e.g., phosphate (e.g., in the form of phosphate (e.g., sodium phosphate (e.g., monobasic and/or dibasic), phosphoric acid, or a combination thereof), borate (e.g., sodium borate (e.g., decahydrate)), or a combination thereof), as appropriate. In some embodiments, the pH of a polymer composition or an extended release otic composition, when gelled, can be measured indirectly, through equilibration with purified distilled water. In some embodiments, a gel formed from a polymer composition or an extended release otic composition as described herein can have a pH of about 5.5 to about 8.5 (e.g., about 5.5 to 8.0, about 5.5 to about 7.5, about 5.5 to about 7.0, about 5.5 to about 6.5, about 5.5 to about 6.0, about 6.5 to about 7.0, about 6.5 to about 7.5, about 6.5 to about 8.0, about 7.0 to about 8.5, about 7.5 to about 8.5, or about 8.0 to about 8.5). In some embodiments, a gel formed from a polymer composition or an extended release otic composition as described herein can have a pH of about 6.0 to about 7.7 (e.g., about 6.0 to about 7.0, about 6.6 to about 7.7, about 6.8 to about 7.7, about 6.6 and about 6.8). In some such embodiments, the pH of the gelled polymer composition or extended release otic composition can be about 6.0 to about 6.5 (e.g., about 6.1 to about 6.4).

In some embodiments, temperature is used to influence the gelation time. In some such embodiments, the polymer composition can be prepared as a chilled composition and/or from one or more chilled components.

The crosslinking density of the gel can be influenced by the overall molecular weight of the crosslinker and functional polymer and the number of functional groups available per molecule. A lower molecular weight of functional polymer, such as about 600 Da, will typically yield higher crosslinking density as compared to a higher molecular weight, such as 10,000 Da. The crosslinking density also can also be influenced by the overall percent solids of the crosslinker and functional polymer solutions. Increasing the percent solids increases the probability that an electrophilic group will combine with a nucleophilic group prior to inactivation by hydrolysis. Yet another method to influence crosslink density is by adjusting the stoichiometry of nucleophilic groups to electrophilic groups. A one to one ratio generally leads to the highest crosslink density.

### Excipients

Extended release otic compositions or polymer compositions as described herein can contain excipients such as pH buffers, tonicity agents, mucoadhesive agents, stabilizing agents, preservatives, carriers, and penetration enhancers. In some embodiments, excipients that can be incorporated into the polymer compositions or extended release otic compositions include diluents, buffers, dispersing agents or viscosity modifying agents, solubilizers, stabilizers, and osmolarity modifying agents.

The term “diluent” refers to chemical compounds that can be used to dilute a component (e.g., a functional polymer, crosslinker, and/or active agent) of a polymer composition or extended release otic composition (e.g., prior to delivery). In some embodiments, a diluent is with the auris media and/or auris interna.

The term “dispersing agents,” and/or “viscosity modulating agents” and/or “thickening agents” refer to materials that enhance dispersion of particulate matter in a solution or modify the viscosity of a solution or suspension. Examples of dispersing agents/materials include, but are not limited to, hydrophilic polymers, electrolytes, TWEEN® 60 or TWEEN® 80, PEG, polyvinylpyrrolidone (PVP; also known as povidone and commercially known as Kollidon®, and PLASDONE®), and the carbohydrate-based dispersing agents such as, for example, modified celluloses such as hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose, carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), and polyethylene glycol, having a molecular weight of about 300 to about 6000, or about 3350 to about 4000, or about 7000 to about 5400. In some embodiments, the amount of thickening agent is about 1%, 5%, about 10%, or about 15% of the total weight of the composition. In some instances, dispersants improve composition physical stability by inhibiting drug crystallization.

The term “solubilizer” refers to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins and other cyclodextrins, ethanol, n-butanol, isopropyl alcohol, cholesterol, bile salts, polyethylene glycol 200-600, glycofurol, TRANSCUTOL®, propylene glycol, and dimethyl isosorbide, ethanol, and other organic solvents. Amphiphilic molecules such as poloxamers and tweens can also serve as solubilizers. In some embodiments, sustained reservoirs of solubilizer can be obtained when these solubilizers are at concentrations above the critical micelle concentration (CMC) or above their solubility (e.g. bile salts). In some embodiments, a solubilizer includes one or more of propylene glycol, PEG300, ethanol, and cyclodextrins, poloxamer 407 and poloxamer 188.

Various physical or viscosity modifiers can be used to enhance mechanical strength and stability of a polymer composition or an extended release otic composition. In some embodiments, particles (e.g., microparticles or nanoparticles) of an active agent can be used to increase mechanical stability of the hydrogel. Particles may be suspended in the hydrogel, or covalently or coupled to the polymer by ionic or hydrophobic interactions.

In some embodiments, other viscosity modifiers, stabilizers, and/or permeation enhancers can be included in a polymer composition or an extended release otic composition. Non-limiting examples of viscosity modifiers include polymers such as dextrans or other polysaccharides, PLURONICs (also referred to as poloxamers, are a class of synthetic block copolymers which consist of hydrophilic poly(ethylene oxide) (PEO) and hydrophobic poly(propylene oxide) (PPO), arranged in an A-B-A triblock structure, thus giving PEO-PPO-PEO), emulsifiers and micelles. Micelles of permeation enhancers and/or solubilizers can, in some embodiments, be used to achieve sustained concentrations in the formulation. In some embodiments, micelles of Poloxamer 407 and/or Poloxamer 188 can be used as a solubilizer, for example at a concentration of about 1% to about 10% (e.g., about 1% to about 2%, about 1% to about 3%, about 1% to about 5%, about 1% to about 8%, about 2% to about 10%, about 3% to about 10%, about 5% to about 10%, or about 8% to about 10%) by weight of the polymer composition or extended release otic composition. In some embodiments, the concentration of a poloxamer does not contribute to gel formation or create high viscosity. In some embodiments, a polymer composition or extended release otic composition as described herein does not include a poloxamer.

In some instances, an active agent may be present in a metastable solid form, such as an amorphous particle, a polymorph, or a salt form where a different solid form has lower solubility; e.g., a free base crystalline form. In some embodiments, an excipient such as polyvinylpyrrolidone and poloxamer 407 may serve as a solubilizer and as a dispersant to inhibit drug crystallization.

The term “stabilizer” refers to compounds such as antioxidants, buffers, acids, and preservatives that are typically compatible with the environment of the auris media and/or auris interna. Stabilizers can include agents that improve the compatibility of excipients with a container, or a delivery system, including a syringe or a glass bottle, that improves the stability of a component of an extended release otic composition or polymer composition, or improve composition stability, for example, to avoid a change in phase.

A tonicity agent can be included in some embodiments in order to achieve a particular tonicity. In general, the endolymph has a higher osmolality than the perilymph. For example, the endolymph has an osmolality of about 304 mOsm/kg H₂O, while the perilymph has an osmolality of about 294 mOsm/kg H₂O.

In some forms, the polymer compositions or extended release otic compositions described herein provide an osmolality of about 100 mOsm/kg to about 1000 mOsm/kg, (e.g., about 200 mOsm/kg to about 400 mOsm/kg, about 240 mOsm/kg to about 350 mOsm/kg, about 250 mOsm/kg to about 350 mOsm/kg, about 270 mOsm/kg to about 320 mOsm/kg, about 280 mOsm/kg to about 320 mOsm/kg, about 100 mOsm/kg to about 200 mOsm/kg, about 100 mOsm/kg to about 300 mOsm/kg, about 100 mOsm/kg to about 500 mOsm/kg, about 100 mOsm/kg to about 700 mOsm/kg, about 100 mOsm/kg to about 900 mOsm/kg, about 200 mOsm/kg to about 1000 mOsm/kg, about 300 mOsm/kg to about 1000 mOsm/kg, about 500 mOsm/kg to about 1000 mOsm/kg, about 700 mOsm/kg to about 1000 mOsm/kg, about 900 mOsm/kg to about 1000 mOsm/kg, or about 300 to about 600 mOsmol/kg). In some forms, the polymer compositions or extended release otic compositions described herein provide an osmolality of about 550 mOsm/kg to about 600 mOsm/kg, (e.g., about 560 to about 590 mOsm/kg). In some forms, the polymer compositions or extended release otic compositions described herein have an osmolality of about 280 mOsm/kg. In some forms, the polymer compositions or extended release otic compositions described herein have an osmolarity of about 100 mOsm/L to about 1000 mOsm/L (e.g., about 200 mOsm/L to about 400 mOsm/L, about 240 mOsm/L to about 350 mOsm/L, about 250 mOsm/L to about 350 mOsm/L, about 270 mOsm/L to about 320 mOsm/L, about 280 mOsm/L to about 320 mOsm/L, about 100 mOsm/L to about 200 mOsm/L, about 100 mOsm/L to about 300 mOsm/L, about 100 mOsm/L to about 500 mOsm/L, about 100 mOsm/L to about 700 mOsm/L, about 100 mOsm/L to about 900 mOsm/L, about 200 mOsm/L to about 1000 mOsm/L, about 300 mOsm/L to about 1000 mOsm/L, about 500 mOsm/L to about 1000 mOsm/L, about 700 mOsm/L to about 1000 mOsm/L, or about 900 mOsm/L to about 1000 mOsm/L). In some forms, the osmolarity of the composition is designed such that the gel is hypotonic with the targeted otic structure (e.g., the endolymph, perilymph, or the like). In some forms, the osmolarity of the composition is designed such that the gel is isotonic with the targeted otic structure (e.g., endolymph, perilymph, or the like). In some forms, the osmolarity of the composition is designed such that the gel is hypertonic with the targeted otic structure (e.g., the endolymph, perilymph, or the like).

Osmolarity/osmolality can be adjusted, for example, by the use of appropriate salt concentrations (e.g., concentration of potassium salts) or the use of tonicity agents, which renders the compositions endolymph-compatible and/or perilymph-compatible (e.g., the gel is isotonic with the endolymph and/or perilymph). In some instances, endolymph-compatible and/or perilymph-compatible extended release otic compositions or polymer compositions can cause minimal disturbance to the environment of the inner ear and cause minimum discomfort (e.g., vertigo and/or nausea) to a subject (e.g., a mammal) upon administration.

In some forms, a gel formed by an extended release otic composition or polymer composition can be isotonic with the perilymph. Isotonic compositions can, in some cases, be formed by the inclusion of a tonicity agent in an extended release otic composition or polymer composition. Suitable tonicity agents include, but are not limited to, any pharmaceutically acceptable sugar, salt or any combinations or mixtures thereof, such as, but not limited to dextrose, glycerin, mannitol, sorbitol, sodium chloride, and other electrolytes. Sodium chloride or other tonicity agents can be optionally used to adjust tonicity, if necessary. Representative salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. In the case of phosphate buffers, it will be understood that sodium phosphate monobasic and sodium phosphate dibasic are typically used in combination to achieve a particular pH, and collectively, they can be called “sodium phosphate”. In some cases, for example, when borate is also used, phosphoric acid can be used to further alter the pH. In some embodiments, a tonicity agent can be is sodium chloride.

In some embodiments, a gel formed by an extended release otic composition or polymer composition can be slightly hypotonic so that water is pulled from the formulation into the tissue to increase contact and adhesion with the tissue.

Extended release otic compositions or polymer compositions can, in some embodiments, include one or more pH-adjusting agents or buffering agents. Non-limiting examples of pH adjusting agents or buffers include acetate, bicarbonate, ammonium chloride, citrate, phosphate, borate, pharmaceutically acceptable salts thereof and combinations or mixtures thereof. Non-limiting examples water-soluble buffering agents are alkali or alkaline earth metal carbonates, phosphates, bicarbonates, citrates, borates, acetates, succinates and the like, such as sodium phosphate, citrate, borate, acetate, bicarbonate, carbonate and HEPES. In some embodiments, Tromethamine (TRIS) is not used in polymer compositions or extended release otic compositions where a primary amine is a functional group.

In some embodiments, an extended release otic composition or polymer composition can include sodium borate decahydrate in an amount of about 0.01% to about 3.0% (e.g., about 0.01% to about 0.1%, about 0.01% to about 0.5%, about 0.01% to about 1.0%, about 0.01% to about 2.0%, about 0.1% to about 3.0%, about 0.5% to about 3.0%, about 1.0% to about 3.0%, about 2.0% to about 3.0%, about 0.05% to about 2.0%, or about 0.5% to about 1.5%) by weight. In some embodiments, an extended release otic composition or polymer composition can include sodium borate decahydrate in an amount of about 0.05% to about 2.0% (e.g., about 0.5% to about 1.5%, or about 1.2%) by weight.

In some embodiments, an extended release otic composition or polymer composition can include sodium phosphate in an amount of about 0.01% to about 3.0% (e.g., about 0.01% to about 0.1%, about 0.01% to about 0.5%, about 0.01% to about 1.0%, about 0.01% to about 2.0%, about 0.1% to about 3.0%, about 0.5% to about 3.0%, about 1.0% to about 3.0%, about 2.0% to about 3.0%, about 0.05% to about 2.0%, or about 0.5% to about 1.5%) by weight. In some embodiments, an extended release otic composition or polymer composition can include sodium phosphate in an amount of about 0.05% to about 2.0% (e.g., about 0.5% to about 1.5%, or about 1.1%) by weight.

In some embodiments, an extended release otic composition or polymer composition can include phosphoric acid in an amount of about 0.01% to about 3.0% (e.g., about 0.01% to about 0.1%, about 0.01% to about 0.5%, about 0.01% to about 1.0%, about 0.01% to about 2.0%, about 0.1% to about 3.0%, about 0.5% to about 3.0%, about 1.0% to about 3.0%, about 2.0% to about 3.0%, about 0.05% to about 2.0%, or about 0.5% to about 1.5%) by weight. In some embodiments, an extended release otic composition or polymer composition can include phosphoric acid in an amount of about 0.05% to about 2.0% (e.g., about 0.5% to about 1.5%, or about 0.9%) by weight.

In some forms, the compositions include a mucoadhesive. In some cases, a mucoadhesive facilitates adhesion to a portion of the ear, such as the round window membrane. Mucoadhesive agents include, but are not limited to, carbomers, such as CARBOPOL® 934P, polyvinylpyrrolidone polymer (PVP); a water-swellable, but water-insoluble, cross-linked carboxy-functional polymer; a crosslinked poly(acrylic acid) (e.g. CARBOPOL® 947P); a carbomer homopolymer; a carbomer copolymer; a hydrophilic polysaccharide gum; maltodextrin; a cross-linked alginate gum gel, hydroxypropyl methylcellulose, and a water-dispersible polycarboxylated vinyl polymer. Mucoadhesive agents are described in U.S. Pat. No. 8,828,980 to Lichter, et al, incorporated herein by reference in its entirety.

Examples of surfactants include, but are not limited to, sodium lauryl sulfate, sodium decussate, TWEEN® 60 (polyethylene glycol sorbitan monostearate) or TWEEN® 80 (polyethylene glycol sorbitan monooleate), triacetin, D-a-tocopheryl polyethylene glycol succinate (vitamin E TPGS), phospholipids, lecithins, phosphatidyl cholines (c8-c18), phosphatidylethanolamines (c8-c18), phosphatidylglycerols (c8-c18), sorbitan monooleate, polyoxyethylene sorbitan monooleate, polysorbates, bile salts, and glyceryl monostearate, Extended release otic compositions or polymer compositions can, in some embodiments, include penetration enhancers that allow for delivery of the active agents across a barrier, such as the oval window or the round window of the ear. Typically, the penetration enhancers are auris-compatible. Penetration enhancers include sodium lauryl sulfate, sodium octyl sulfate, sodium dodecyl sulfate, octyl-trimethyl-ammonium bromine, dodecyl-trimethyl ammonium bromide, sodium laurate, polyoxyethylene-20-cetyl ether, laureth-9, sodium dodecylsulfate, dioctyl sodium sulfosuccinate, polyoxyethylene-9-lauryl ether (PLE), TWEEN® 20, TWEEN® 80, nonylphenoxypolyethylene (NP-POE), polysorbates, bile salts, fatty acids and derivatives, chelating agents (such as EDTA, citric acid, and salicylates, sulfoxides (such as dimethyl sulfoxide (DMSO) and decylmethyl sulfoxide), and alcohols (such as ethanol, isopropanol, glycerol, and propanediol. In some cases, permeation enhancers are partially soluble fatty acids such as oleic acid present at concentrations high enough to form a suspension and, hence, micro-reservoir for sustained presence of permeation enhancer. In some embodiments, an extended release composition includes a permeation enhancer that is depleted after providing an initial higher release of an active agent.

In some forms, extended release otic compositions or polymer compositions can include a preservative. Exemplary preservatives are also described in U.S. Pat. No. 8,828,980 to Lichter, et al, herein incorporated by reference in its entirety. Suitable preservatives include, but are not limited to, benzoic acid, boric acid, p-hydroxybenzoates, alcohols, quaternary compounds, stabilized chlorine dioxide, mercurials, such as merfen and thiomersal, or a combination thereof. In some embodiments, a preservative can include butylated hydroxytoluene (BHT). In some embodiments, an extended release otic composition or polymer composition can include BHT in an amount of about 0% to about 0.01% (e.g., about 0.0005% to about 0.01%, about 0.001% to about 0.01%, or about 0.005% to about 0.01%). In some embodiments, a preservative can include butylated hydroxytoluene (BHT). In some embodiments, an extended release otic composition or polymer composition can include BHT in an amount of about 0% to about 0.01% (e.g., about 0.0005% to about 0.01%, about 0.001% to about 0.01%, or about 0.005% to about 0.01%). In some embodiments, an extended release otic composition or polymer composition can include BHT in an amount of about 0% to about 0.005% (e.g., about 0.001% to about 0.003% or about 0.002%).

### III. Administration of the Polymers and Crosslinking

Polymer compositions or extended release otic compositions can be administered using any appropriate method.

Also provided herein are methods of preparing a polymer composition or an extended release otic composition. In some embodiments, a polymer composition or an extended release otic composition can be prepared by: combining a solution or suspension of a functional polymer and a solution or suspension of a crosslinker. In some embodiments, a solution or suspension of a functional polymer and a solution or suspension of a crosslinker are combined during administration of the polymer composition or extended release otic composition (e.g., when using a dual syringe apparatus). In some embodiments, a polymer composition or an extended release otic composition can be prepared by: combining a solution or suspension of a functional polymer and a solution or suspension of a crosslinker, such that the combination has a pH of about 5.5 to about 8.5.

In some cases, the functional polymer is a solid (e.g., a lyophilized powder) and is reconstituted at or near time of use. Accordingly, in some embodiments, a polymer composition or an extended release otic composition can be prepared by: (a) making a solution or suspension of the functional polymer, (b) making a solution or suspension of the crosslinker, and (c) combining the solution or suspension of the functional polymer and the solution or suspension of the crosslinker. In some cases, the crosslinker is provided as a solution. Accordingly, in some embodiments, a polymer composition or an extended release otic composition can be prepared by: (a) making a solution or suspension of the functional polymer, and (b) combining the solution or suspension of the functional polymer with a solution or suspension of the crosslinker. In some embodiments, a polymer composition or an extended release otic composition can be prepared by: (a) making a solution or suspension of the functional polymer, (b) altering the pH of a solution or suspension of the crosslinker, and (c) combining the solution or suspension of the functional polymer with the solution or suspension of the crosslinker. In some embodiments, making a solution or suspension of the functional polymer or of the crosslinker can include adjusting the pH of the solution or suspension. In some embodiments, adjusting the pH of a solution or a suspension of the functional polymer can include adjusting the pH to about 1.6 to about 4.0. In some embodiments, adjusting the pH of a solution or suspension of the crosslinker can include adjusting the pH to about 5.5 to about 8.5. In some embodiments, a solution or suspension of a functional polymer and a solution or suspension of a crosslinker are combined during administration of the polymer composition or extended release otic composition (e.g., when using a dual syringe apparatus).

In some cases, the functional polymer is provided as a solution or suspension. Accordingly, in some embodiments, a polymer composition or an extended release otic composition can be prepared by: (a) making a solution or suspension of the crosslinker, and (b) combining the solution or suspension of the functional polymer and the solution or suspension of the crosslinker. In some cases, the crosslinker is provided as a solution. In some embodiments, a polymer composition or an extended release otic composition can be prepared by combining the solution or suspension of the functional polymer with a solution or suspension of the crosslinker. In some embodiments, a polymer composition or an extended release otic composition can be prepared by: (a) making a solution or suspension of the crosslinker, (b) altering the pH of a solution or suspension of the functional polymer, and (c) combining the solution or suspension of the functional polymer with the solution or suspension of the crosslinker. In some embodiments, making a solution or suspension of the functional polymer or of the crosslinker can include adjusting the pH of the solution or suspension. In some embodiments, adjusting the pH of a solution or a suspension of the functional polymer can include adjusting the pH to about 1.6 to about 4.0. In some embodiments, adjusting the pH of a solution or suspension of the crosslinker can include adjusting the pH to about 5.5 to about 8.5. In some embodiments, a solution or suspension of a functional polymer and a solution or suspension of a crosslinker are combined during administration of the polymer composition or extended release otic composition (e.g., when using a dual syringe apparatus).

In some embodiments, an extended release otic composition can be prepared from components: (i) a solid form (e.g., powder) of an active agent (e.g., dexamethasone), (ii) a diluent solution, (iii) a solid form (e.g., powder) of a functional polymer (e.g., NHS-PEG), and (iv) a solution of a crosslinker (e.g., trilysine). In some embodiments, an extended release otic composition can be prepared by (a) combining an active agent (e.g., dexamethasone) with a solution of a crosslinker (e.g., trilysine) to form a first mixture, (b) combining the functional polymer with the diluent solution to form a second mixture, and (c) combining the first mixture and the second mixture.

In some embodiments, an active agent can be included in the solution or suspension of the functional polymer; in the solution or suspension of the crosslinker; provided in a separate solution or suspension; provided as a separate solid (e.g., dry powder); provided as a solid (e.g., dry powder) and combined with functional polymer (e.g., prior to forming a solution or suspension of the functional polymer), provided as a solid (e.g., dry powder) and combined with crosslinker (e.g., prior to forming a solution or suspension of the crosslinker), or a combination thereof. In some embodiments where an active agent is provided in a separate solution, suspension, or as a solid (e.g., a dry powder), combining the solution or suspension of the functional polymer with the solution or suspension of the crosslinker can further include combining the solution, suspension, or solid form (e.g., dry powder) of the active agent.

In some embodiments, combining the solution or suspension of the functional polymer with the solution or suspension of the crosslinker occurs less than 60 minutes (e.g., less than 50 minutes, less than 40 minutes, less than 30 minutes, less than 20 minutes, or less than 10 minutes) after making a solution or suspension of the functional polymer.

Also provided herein are methods of administering a polymer composition or an extended release otic composition. In some embodiments, a polymer composition or an extended release otic composition can be administered by (i) preparing a polymer composition or extended release composition, e.g., by any of the methods described herein, and (ii), administering the polymer composition or extended release otic composition to a subject.

In some embodiments, a polymer composition or an extended release otic composition can be administered by (i) preparing a polymer composition or extended release otic composition by combining a solution or suspension of a functional polymer and a solution or suspension of a crosslinker, and (ii), administering the polymer composition or extended release otic composition to a subject. In some embodiments, a polymer composition or an extended release otic composition can be administered by (i) preparing a polymer composition or extended release composition by combining a solution or suspension of a functional polymer and a solution or suspension of a crosslinker, such that the combination has a pH of about 5.5 to about 8.5, and (ii), administering the polymer composition or extended release otic composition to a subject. In some embodiments, the preparing of a polymer composition or extended release otic composition occurs during the administering, for example, when using a dual syringe apparatus containing separate reservoirs of functional polymer and crosslinker.

In some embodiments, a polymer composition or an extended release otic composition can be administered by (i) preparing a polymer composition or extended release otic composition by (a) making a solution or suspension of the functional polymer, (b) making a solution or suspension of the crosslinker, and (c) combining the solution or suspension of the functional polymer and the solution or suspension of the crosslinker, and (ii), administering the polymer composition or extended release otic composition to a subject. In some embodiments, a polymer composition or an extended release otic composition can be administered by (i) preparing a polymer composition or extended release composition by (a) making a solution or suspension of the functional polymer, and (b) combining the solution or suspension of the functional polymer with a solution or suspension of the crosslinker, and (ii), administering the polymer composition or extended release otic composition to a subject. In some embodiments, a polymer composition or an extended release otic composition can be administered by (i) preparing a polymer composition or extended release composition by (a) making a solution or suspension of the functional polymer, (b) altering the pH of a solution or suspension of the crosslinker, and (c) combining the solution or suspension of the functional polymer with the solution or suspension of the crosslinker, and (ii), administering the polymer composition or extended release otic composition to a subject.

In some embodiments, a polymer composition or an extended release otic composition can be administered by (i) preparing a polymer composition or extended release composition by (a) making a solution or suspension of the crosslinker, and (b) combining the solution or suspension of the crosslinker with a solution or suspension of the functional polymer, and (ii), administering the polymer composition or extended release otic composition to a subject. In some embodiments, a polymer composition or an extended release otic composition can be administered by (i) preparing a polymer composition or extended release composition by (a) making a solution or suspension of the crosslinker, (b) altering the pH of a solution or suspension of the functional polymer, and (c) combining the solution or suspension of the functional polymer with the solution or suspension of the crosslinker, and (ii), administering the polymer composition or extended release otic composition to a subject.

In some embodiments of administering a polymer composition or an extended release otic composition, an active agent can be included in the solution or suspension of the functional polymer; in the solution or suspension of the crosslinker; provided in a separate solution or suspension; provided as a separate solid (e.g., dry powder); or provided as a solid (e.g., dry powder) combined with functional polymer (e.g., prior to forming a solution or suspension of the functional polymer), or a combination thereof. In some embodiments where an active agent is provided in a separate solution, suspension, or as a solid (e.g., a dry powder), combining the solution or suspension of the functional polymer with the solution or suspension of the crosslinker can further include combining the solution, suspension, or solid form (e.g., dry powder) of the active agent.

In some embodiments, combining the solution or suspension of the functional polymer with the solution or suspension of the crosslinker occurs less than 60 minutes (e.g., less than 50 minutes, less than 40 minutes, less than 30 minutes, less than 20 minutes, or less than 10 minutes) after making a solution or suspension of the functional polymer. In some embodiments, step (ii) occurs less than 10 minutes (e.g., less than 9 minutes, less than 8 minutes, less than 7 minutes, less than 6 minutes, less than 5 minutes, less than 4 minutes, less than 3 minutes, less than 2 minutes, or less than 1 minute) after combining the solution or suspension of the functional polymer with the solution or suspension of the crosslinker.

In the case of intrinsic gelation due to the presence of crosslinking agents, it can be important to control the timing of chemical crosslinking and administration. It will be appreciated that the timing of step (ii) is related to the gelation time of the extended release otic composition or polymer composition as described hereinabove. Typically, the timing of step (ii) is such that administration of the extended release otic composition or polymer composition is not yet a gel.

In some embodiments, gelation can be initiated upon application of an external factor, such as light or other external energy before, during, or after administration. In some embodiments, a polymer composition or an extended release otic composition can be administered to a subject followed by initiation of the crosslinking reaction.

Extended release otic compositions can be administered to the middle ear of a subject in need thereof, for example, by trans-tympanic injection. In some embodiments, the extended release otic compositions are administered on or near the round window membrane via trans-tympanic injection. Extended release otic compositions, in some embodiments, may also be administered on or near the round window or the crista fenestrae cochleae through entry via a post-auricular incision and surgical manipulation into or near the round window or the crista fenestrae cochleae area. In some embodiments, when administered to the ear of a subject, the extended release otic composition does not engulf any of the ossicles. In some embodiments, when administered to the ear of a subject, the extended release otic composition does not contact any of the ossicles.

In some cases, administering can include using a syringe and small diameter needle, (e.g., 23 G to 30 G or smaller), wherein the needle is inserted through the tympanic membrane and guided to the area of the round window or crista fenestrae cochleae. The composition is then deposited on or near the round window or crista fenestrae cochleae. In some embodiments, when being administered, an extended release otic composition is a liquid. In some embodiments, before administration, an extended release otic composition is not exposed to a temperature above about 26° C. In some embodiments, during administration, the extended release otic composition has a temperature of about 20° C. to about 25° C.

In some embodiments, an extended release otic composition can also be administered into the tympanic cavity or applied on the tympanic membrane or onto or in the auditory canal by injection, direct instillation or perfusion of the inner ear compartments, or in surgical procedures including, cochleostomy, labyrinthotomy, mastoidectomy, stapedectomy, or endolymphatic sacculotomy.

In some embodiments, the administering can include administering a therapeutically effective dose. In some embodiments, the administering can include administering a prophylactically effective dose. In some embodiments, administering can include administering about 5 to about 500 microliters (e.g., about 5 μL to about 400 μL, about 5 μL to about 300 μL, about 5 μL to about 200 μL, about 5 μL to about 100 μL, about 5 μL to about 50 μL, about 5 μL to about 25 μL, about 5 μL to about 10 μL, about 10 μL to about 500 μL, about 25 μL to about 500 μL, about 50 μL to about 500 μL, about 100 μL to about 500 μL, about 200 μL to about 500 μL, about 300 μL to about 500 μL, about 400 μL to about 500 μL, about 25 μL to about 300 μL, about 50 μL to about 200 μL, about 30 μL to about 70 μL, or about 40 μL to about 60 μL). In some embodiments, administering can include administering about 50 μL, about 100 μL, or about 200 μL.

In some embodiments, the administering can include administering 3 mg dexamethasone in a volume of 50 μL of an extended release otic composition including 6% by weight of dexamethasone.

In some cases, a gel formed from a polymer composition or extended release otic composition as described herein can exhibit a swelling of less than about 100% (e.g., less than less than about 80%, less than about 70%, less than about 60%, less than about 50%, or less than about 40%), within 1 day of being administered to the ear of a subject.

In some embodiments, administering can include the use of a particular instrument, such as an inline mixer (sometimes also called a static mixer) downstream of a dual syringe injector, which can, in some cases, mix the components and minimize the time between initial mixing and administration to the target site. In some embodiments, methodologies and devices for performing in situ gelation, developed for other adhesive or sealant systems such as fibrin glue or sealant applications, may be used with the polymer compositions or extended release otic compositions described herein. See, for example, U.S. Pat. Nos. 4,874,368; 4,631,055; 4,735,616; 4,359,049; 4,978,336; 5,116,315; 4,902,281; 4,932,942; PCT WO 91/09641; and R. A. Tange, “Fibrin Sealant” in Operative Medicine: Otolaryngology, volume 1 (1986), each of which is herein incorporated by reference in its entirety.

In some embodiments, before administering a polymer composition or an extended release otic composition as provided herein, an anesthetic can be applied to the ear drum and/or ear canal of the subject. For example, before administering, an anesthetic (e.g., EMLA® cream) can be applied to the ear drum and/or ear canal of the subject about 5 minutes to about 1 hour (e.g., about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 20 minutes to about 1 hour, about 30 minutes to about 1 hour, about 40 minutes to about 1 hour, or about 50 minutes to about 1 hour) before the administering. In some embodiments, before administering, an anesthetic (e.g., EMLA® cream) can be applied to the ear drum and/or ear canal of the subject immediately before the administering.

In some embodiments, administration can be visualized, for example, using an endoscope. Without being bound by any particular theory, it is believed that visualization can help with placement of an extended release otic composition in a desired location (e.g., on the round window membrane) and/or help to avoid placement of an extended release otic composition at an undesired location (e.g., engulfing one or more ossicles).

In some embodiments, an extended release otic composition can be administered in a single dose or in multiple doses. Certain factors may influence the dosage required to effectively treat or prevent a disorder, including, but not limited to, the severity of the disease or disorder, previous preventions, the general health and/or age of the subject, and other diseases present. It will also be appreciated that the effective dosage of the extended release otic composition used for prevention may increase or decrease over the course of a particular prevention. Changes in dosage may result and become apparent from the results of assays.

Before, during or after administration, polymer compositions or extended release otic composition effects a transition from a liquid state to a gel state. In some embodiments, the gel provides a therapeutically effective concentration of an active agent for a period of between about 5 days to about 6 months (e.g., about 5 days to about 1 week, about 5 days to about 2 weeks, about 5 days to about 3 weeks, about 5 days to about 1 month, about 5 days to about 2 months, about 5 days to about 3 months, about 5 days to about 4 months, about 5 days to about 5 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, about 2 weeks to about 2 months, or about 1 month to about 3 months) In some embodiments, the gel provides a therapeutically effective concentration of an active agent for at least 1 week (e.g., at least about 2 weeks, 3 weeks, 1 month, 2 months, 3 months, 4 months, 5 months, or 6 months).

In some cases, the subject to be treated is an adult or pediatric human undergoing treatments that can cause hearing loss, such as chemotherapy, hearing loss due to aging, hearing loss due to repeated exposure to loud noises, and other disorders damaging the cilia in the inner ear such as autoimmune disorders, infection, excess fluid or pressure.

In some embodiments, the subject to be treated is an adult human. In some embodiments, the subject to be treated has a diagnosis of unilateral Definite Meniere's disease defined by the Classification Committee of the Bárány Society or the American Academy of Otolaryngology-Head and Neck Surgery (AAO-HNS). In some embodiments, the subject to be treated has reported two or more episodes of definitive vertigo (lasting 20 minutes or more) in the month prior to screening for administration of an extended release otic composition. In some embodiments, the subject has documented acquired asymmetric sensorineural hearing loss, as reported by the patient or documented by audiometric testing. In some embodiments, a subject is not administered an extended release otic composition as provided herein if the subject has ongoing chronic inflammatory or infectious middle ear disease. In some embodiments, a subject is not administered an extended release otic composition as provided herein if the subject has an active infection in the ear, sinuses, or upper respiratory system. In some embodiments, a subject is not administered an extended release otic composition as provided herein if the subject has current tympanic membrane perforation. In some embodiments, a subject is not administered an extended release otic composition as provided herein if the subject has active benign paroxysmal positional vertigo (BPPV) symptoms. In some embodiments, a subject is not administered an extended release otic composition as provided herein if the subject has a history of superior canal dehiscence. In some embodiments, a subject is not administered an extended release otic composition as provided herein if the subject has a history of drop attacks (Tumarkin's Otolithic Crisis). In some embodiments, a subject is not administered an extended release otic composition as provided herein if the subject has a history of vestibular migraine. In some embodiments, a subject is not administered an extended release otic composition as provided herein if the subject has a history of endolymphatic sac surgery. In some embodiments, a subject is not administered an extended release otic composition as provided herein if the subject has a history of middle ear surgery (other than tympanostomy tubes). In some embodiments, a subject is not administered an extended release otic composition as provided herein if the subject has retrocochlear pathology affecting the auditory or vestibular systems (e.g., acoustic neuroma, multiple sclerosis). In some embodiments, a subject is not administered an extended release otic composition as provided herein if the subject has a significant abnormality of the ear canal or tympanic membrane that would preclude IT injection. In some embodiments, a subject is not administered an extended release otic composition as provided herein if the subject has history of immunodeficiency disease or autoimmune disease.

Accordingly, provided herein are methods of treating a subject with an otic disease or disorder. In some embodiments, provided herein are methods of treating a subject with an otic disease or disorder including administering a therapeutically effective dose of an extended release otic composition as described herein. In some embodiments, provided herein are methods of treating a subject with an otic disease or disorder including administering a therapeutically effective dose of an extended release otic composition as described herein to an ear of a subject in need thereof. In some embodiments, provided herein are methods of treating a subject with an otic disease or disorder including (i) identifying the subject as having the otic disease or disorder, and (ii) administering a therapeutically effective dose of an extended release otic composition as described herein to an affected ear of the subject. In some embodiments, the otic disease or disorder can be selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.

In addition, provided herein are methods of treating Ménière's Disease, the methods including administering to an ear of a subject in need thereof a therapeutically effective dose of an extended release otic composition as described herein. Also provided are methods of treating Ménière's Disease, the methods including (i) identifying a subject as having Ménière's Disease; and (ii) administering a therapeutically effective dose of an extended release otic composition as described herein to an affected ear of the subject.

In some embodiments, prior to administering an extended release otic composition, the subject is evaluated. For example, prior to administering an extended release otic composition, the subject can be evaluated for a baseline level of endolymphatic hydrops, perilymphatic enhancement, or both, for example, as assessed by delayed intravenous gadolinium contrast enhanced 3T MRI. In some cases, the subject is evaluated for a baseline level of endolymphatic hydrops. In some embodiments, the subject is evaluated for a baseline level of perilymphatic enhancement. In some embodiments, the subject is evaluated for a baseline level of both endolymphatic hydrops and perlymphatic enhancement. In some embodiments, prior to administering an extended release otic composition, the subject can be evaluated for a baseline level of severity and/or frequency of vertigo episodes. In some embodiments, prior to administering an extended release otic composition, the hearing of the subject can be evaluated for a baseline (e.g., using audiometric evaluation). In some embodiments, prior to administering an extended release otic composition, the subject can be evaluated for a baseline dizziness handicap inventory (DHI) score and/or a tinnitus handicap inventory (THI) score. In some cases, the subject is evaluated on the same day that the extended release otic composition is administered. In some cases, the subject is evaluated about 1 day to about 6 weeks (e.g., about 1 day to about 1 week, about 1 day to about 2 weeks, about 1 day to about 3 weeks, about 1 day to about 4 weeks, about 1 day to about 5 weeks, about 1 week to about 6 weeks, about 3 weeks to about 6 weeks, about 4 weeks to about 6 weeks, about 5 weeks to about 6 weeks, or about 3 weeks to about 5 weeks) before the extended release otic composition is administered.

In some embodiments, the subject is evaluated after administering an extended release otic composition. For example, after administering an extended release otic compositions, the subject can be evaluated for a level of endolymphatic hydrops, perilymphatic enhancement, or both, for example, as assessed by delayed intravenous gadolinium contrast enhanced 3T MRI. In some cases, the subject is evaluated for a level of endolymphatic hydrops. In some embodiments, the subject is evaluated for a level of perilymphatic enhancement. In some embodiments, the subject is evaluated for a level of both endolymphatic hydrops and perlymphatic enhancement. In some embodiments, the subject can be evaluated for a level of severity and/or frequency of vertigo episodes. In some embodiments, the hearing of the subject can be evaluated (e.g., using audiometric evaluation). In some embodiments, the subject can be evaluated for a dizziness handicap inventory (DHI) score and/or a tinnitus handicap inventory (THI) score. In some cases, the subject is evaluated about 1 week to about 4 weeks (e.g., about 1 week to about 2 weeks, about 1 week to about 3 weeks, about 2 weeks to about 4 weeks, or about 3 weeks to about 4 weeks) after the extended release otic composition is administered. In some cases, the subject is evaluated about 2 weeks after the extended release otic composition is administered.

In some embodiments, the subject exhibits an improvement in one or more evaluations after administration of an extended release otic composition as provided herein. For example, in some embodiments, the subject can exhibit an improvement in a level of endolymphatic hydrops, perilymphatic enhancement, or both, for example, as assessed by delayed intravenous gadolinium contrast enhanced 3T MRI. In some cases, the subject can exhibit an improvement in a level of endolymphatic hydrops, e.g., compared to a baseline level. In some embodiments, the subject can exhibit an improvement in a level of perilymphatic enhancement, e.g., compared to a baseline level. In some embodiments, the subject can exhibit an improvement in a level of both endolymphatic hydrops and perlymphatic enhancement, e.g., compared to baseline levels. In some embodiments, the subject can exhibit an improvement in a level of severity and/or frequency of vertigo episodes, e.g., compared to a baseline level. In some embodiments, the subject can exhibit an improvement in hearing (e.g., using audiometric evaluation), e.g., compared to a baseline level. In some embodiments, the subject can exhibit an improvement in a dizziness handicap inventory (DHI) score and/or a tinnitus handicap inventory (THI) score, e.g. compared to baseline score(s).

In some embodiments, vertigo episodes can be evaluated by the subject keeping a daily vertigo diary of severity and frequency of vertigo episodes. In some embodiments, post-administration average vertigo severity and frequency (e.g., averaged over the previous 4 weeks) can be compared to baseline vertigo severity and frequency (e.g., averaged over the 4 weeks prior to administration).

In some embodiments, hearing can be assessed by audiometric examination. In some embodiments, hearing can be assessed using pure tone audiometry at 125, 250, 500, 1000, 2000, 4000 and 8000 Hz and/or word recognition score. In some embodiments, a change from baseline in hearing by pure tone audiometry at 125, 250, 500, 1000, 2000, 4000 and 8000 Hz and word recognition score can be characterized using descriptive statistics.

In some embodiments, change in patient DHI and THI scores from baseline to a post-administration evaluation (e.g., on about Day 29 and/or about Day 85) can be evaluated and characterized using descriptive statistics.

For subjects who receive MRIs, change from baseline in endolymphatic hydrops and perilymphatic enhancement as assessed by delayed intravenous gadolinium contrast enhanced 3T MRI scan can be evaluated post-administration (e.g., on about Day 15).

The amount and extent of distribution of a polymer composition or extended release otic composition in the middle ear of a subject post-administration (e.g., on about Day 15) can also be evaluated.

### IV. Otic Diseases and Disorders

Otic disorders with underlying microvascular etiology, including Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and similar ear disorders have no treatment, other than for some of the symptoms, nor a cure.

Ménière's Disease is a chronic, incurable inner ear disorder with recurrent debilitating symptoms that affect hearing and balance. It is named for French physician Prosper Meniere who, in 1861, first identified and described the symptoms of this medical condition. Researchers are unsure of what causes the buildup of fluid in the inner ear that results in MD. Some believe it is related to vascular insufficiencies, others say it might be due to autoimmune conditions, viral infections, allergic reactions or that the disease may initiate from a trauma. MD appears to have a hereditary component, so a gene mutation may be connected to the regulation of inner ear fluid.

Autoimmune Inner Ear Disease is a rare disorder, appearing in both adults and children, caused by an immune system response. The inner ear can be the direct target of the immune response, but it can be additionally damaged by a deposition of circulating immune complexes or by systemic immune-mediated diseases. The clinical expression of immune-mediated inner ear disease shows a progressive bilateral and asymmetric SNHL profile. Cochlear symptoms are often associated with vestibular disorders. In about 50% of the AIED patients, hearing loss is also associated with vestibular symptoms, such as imbalance and motion intolerance, ataxia and positional or episodic vertigo.

Sensorineural Hearing Loss is due to impaired ability of the cochlea to effectively transduce pressure waves into neural signaling. SNHL is typically associated with exposure to loud noise, aging, head trauma, exposure to ototoxic drugs, infection, autoimmune disease, Meniere's disease, genetic mutations, and tumors of the auditory nerve.

Noise-induced hearing loss is caused by exposure to loud and/or long-lasting sounds. Hearing loss-may occur from prolonged exposure to loud noises, such as heavy machinery, loud music, airplanes or gunfire. Long, repeated or impulse exposure to sounds at or above 85 decibels can cause hearing loss. NIHL causes damage to the hair cells and/or the auditory nerve.

Symptoms of MD, AIED, SNHL, NIHL and other ear disorders include vertigo, hearing loss, ear ringing (tinnitus), and ear pressure. The vertigo may cause severe nausea and imbalance. Hearing loss may become permanent.

There is no treatment, other than for some of the symptoms, nor a cure. Drugs for motion sickness or nausea may help manage the symptoms.

This disclosure also provides methods of treating otic diseases and disorders using a tyrosine kinase inhibitor (e.g., a VEGF inhibitor). Accordingly, provided herein is a method of treating an otic disease or disorder in a subject, the method including identifying a subject as having an otic disease or disorder, and administering a therapeutically effective amount of tyrosine kinase inhibitor to the subject.

Non-limiting examples of otic diseases and disorders include Ménière's Disease, autoimmune inner ear disease (AIED), sensorineural hearing loss (e.g., sudden sensorineural hearing loss or sensorineural hearing loss is associated with diabetes), noise-induced hearing loss (NIHL), age-related hearing loss, tinnitus, damaged cilia from an autoimmune disorder, damaged cilia from an infection, damaged cilia from excess fluid or pressure, and hearing loss due to chemotherapy.

In some cases, the tyrosine kinase inhibitor can be a VEGF inhibitor (e.g., any of the VEGF inhibitors described herein. In some embodiments, tyrosine kinase inhibitor is administered in an amount sufficient to reduce edema and lymphatic dysfunction in an affected ear.

A tyrosine kinase inhibitor can be administered in any appropriate form or by any appropriate route. In some embodiments, the tyrosine kinase inhibitor can be administered systemically. In some embodiments, the tyrosine kinase inhibitor can be administered locally (e.g., to the middle or inner ear, for example, by transtympanic injection). In some cases, the tyrosine kinase inhibitor can be provided in the form of a hydrogel. Non-limiting examples of hydrogels are provided in U.S. Pat. Nos. 9,066,865 and 10,561,736, each of which are incorporated herein by reference in their entirety. As another example, a tyrosine kinase inhibitor can be provided in the form of any of the extended release otic compositions described herein.

### EXEMPLARY EMBODIMENTS

Embodiment 1 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and wherein the polymer composition has a gelation time of about 45 seconds to about 60 minutes at a temperature of about 20° C.

Embodiment 2 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and wherein the polymer composition has a gelation time of about 10 seconds to about 30 minutes at a temperature of about 37° C.

Embodiment 3 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein crosslinker comprises a second functional group; and
water,
wherein a crosslinking reaction can occur between the first functional group and the second functional group to form a gel, and wherein the gel, when formed in the middle ear, has a residence time of at least 5 days.

Embodiment 4 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and the gel has a gel duration of at least 5 days at 37° C.

Embodiment 5 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein the polymer composition has a pH of about 5.5 to about 8.5, and wherein a crosslinking reaction can occur between the first functional group and the second functional group to form a gel.

Embodiment 6 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and wherein the gel, following equilibration in phosphate-buffered saline (PBS) for 1 day, swells less than 100%.

Embodiment 7 is a polymer composition comprising:
about 5% to about 15% by weight of the polymer composition amount of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and wherein the gel is elastic.

Embodiment 8 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein a crosslinking reaction can occur between the first functional group and the second functional group to form a gel, and wherein gel is mucoadhesive.

Embodiment 9 is a polymer composition comprising:
about 5% to about 15% by weight of the polymer composition of a functional polymer, wherein the functional polymer comprises a first functional group;
about 0.05% to about 0.6% by weight of the polymer composition of a crosslinker, wherein the crosslinker comprises a second functional group; and
water,
wherein the polymer composition has a viscosity of about 1 mPa·s to about 1000 mPa·s, and wherein a crosslinking reaction can occur between the first functional group and the second functional group to form a gel.

Embodiment 10 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein the polymer composition has a gelation time of about 45 seconds to about 60 minutes at a temperature of about 20° C.

Embodiment 11 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein the polymer composition has a gelation time of about 10 seconds to about 30 minutes at a temperature of about 37° C.

Embodiment 12 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein the gel, when formed in the middle ear, has a residence time of at least 5 days.

Embodiment 13 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and the gel has a gel duration of at least 5 days at 37° C.

Embodiment 14 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein the polymer composition has a pH of about 5.5 to about 8.5, and wherein a crosslinking reaction can occur between the first functional group and the second functional group to form a gel.

Embodiment 15 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein the gel, following equilibration in phosphate-buffered saline (PBS) for 1 day, swells less than 100%.

Embodiment 16 is a polymer composition comprising:
about 5% to about 15% by weight of the polymer composition amount of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein the gel is elastic.

Embodiment 17 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein a crosslinking reaction can occur between the first functional group and the second functional group to form a gel, and wherein gel is mucoadhesive.

Embodiment 18 is a polymer composition comprising:
about 5% to about 15% by weight of the polymer composition of a functional polymer, wherein the functional polymer comprises a first functional group;
about 0.05% to about 10% by weight of the polymer composition of a crosslinker, wherein the crosslinker comprises a second functional group; and
water,
wherein the first functional group and the second functional group are present in a ratio of about 1.2:0.8 to about 0.8 to 1.2, and
wherein the polymer composition has a viscosity of about 1 mPa·s to about 1000 mPa·s, and wherein a crosslinking reaction can occur between the first functional group and the second functional group to form a gel.

Embodiment 19 is the polymer composition of any one of embodiments 9-16, wherein the polymer composition comprises about 1% to about 10% by weight of the polymer composition of the crosslinker.

Embodiment 20 is the polymer composition of any one of embodiments 9-16, wherein the polymer composition comprises about 1% to about 5% by weight of the polymer composition of the crosslinker.

Embodiment 21 is the polymer composition of any one of embodiments 2-9 or 11-20, wherein the polymer composition has a gelation time of about 45 seconds to about 60 minutes at a temperature of about 20° C.

Embodiment 22 is the polymer composition of any one of embodiments 1, 2, 4-11, or 13-21, wherein the gel, when formed in the middle ear, has a residence time of at least 5 days.

Embodiment 23 is the polymer composition of any one of embodiments 1-3, 5-12, or 14-22, wherein the gel has a gel duration of at least 5 days at 37° C.

Embodiment 24 is the polymer composition of any one of embodiments 1-4, 6-13, or 15-23, wherein the polymer composition has a pH of about 5.5 to about 8.5.

Embodiment 25 is the polymer composition of any one of embodiments 1-5, 7-14, or 15-24, wherein the gel, following equilibration in phosphate-buffered saline (PBS) for 1 day, swells less than 100%.

Embodiment 26 is the polymer composition of any one of embodiments 1-6, 8-15, or 17-25, wherein the gel is elastic.

Embodiment 27 is the polymer composition of any one of embodiments 1-7, 9-16, or 18-26, wherein the gel is mucoadhesive.

Embodiment 28 is the polymer composition of any one of embodiments 1-8, 10-17, or 19-27, wherein the polymer composition has a viscosity of about 1 mPa·s to about 1000 mPa·s.

Embodiment 29 is the polymer composition of any one of embodiments 1-28, wherein the polymer composition comprises about 6% to about 12% by weight of the polymer composition of the functional polymer.

Embodiment 30 is the polymer composition of any one of embodiments 1-28, wherein the polymer composition comprises about 7% to about 10% by weight of the polymer composition of the functional polymer.

Embodiment 31 is the polymer composition of any one of embodiments 1-28, wherein the polymer composition comprises about 8.3% by weight of the polymer composition of the functional polymer.

Embodiment 32 is the polymer composition of any one of embodiments 1-28, wherein the polymer composition comprises about 8% to about 12% by weight of the polymer composition of the functional polymer.

Embodiment 33 is the polymer composition of any one of embodiments 1-28, wherein the polymer composition comprises about 10% by weight of the polymer composition of the functional polymer.

Embodiment 34 is the polymer composition of any one of embodiments 1-33, wherein the polymer composition comprises about 0.05% to about 0.5% by weight of the polymer composition of the crosslinker.

Embodiment 35 is the polymer composition of any one of embodiments 1-33, wherein the polymer composition comprises about 0.1% to about 0.3% by weight of the polymer composition of the crosslinker.

Embodiment 36 is the polymer composition of any one of embodiments 1-33, wherein the polymer composition comprises about 0.2% by weight of the polymer composition of the crosslinker.

Embodiment 37 is the polymer composition of any one of embodiments 1-33, wherein the polymer composition comprises about 0.2% to about 0.6% by weight of the polymer composition of the crosslinker.

Embodiment 38 is the polymer composition of any one of embodiments 1-33, wherein the polymer composition comprises about 0.3% to about 0.5% by weight of the polymer composition of the crosslinker.

Embodiment 39 is the polymer composition of any one of embodiments 1-33, wherein the polymer composition comprises about 0.4% to about 0.6% by weight of the polymer composition of the crosslinker.

Embodiment 40 is the polymer composition of any one of embodiments 1-39, wherein the polymer composition has a gelation time of about 5 minutes to about 20 minutes at a temperature of about 20° C.

Embodiment 41 is the polymer composition of any one of embodiments 1-40, wherein the polymer composition has a gelation time of about 4 minutes to about 12 minutes at a temperature of about 20° C.

Embodiment 42 is the polymer composition of any one of embodiments 1-41, wherein the polymer composition has a gelation time of about 8 minutes to about 12 minutes at a temperature of about 20° C.

Embodiment 43 is the polymer composition of any one of embodiments 1, 2-11, or 13-42, wherein the polymer composition has a gelation time of about 10 seconds to about 30 minutes at a temperature of about 37° C.

Embodiment 44 is the polymer composition of any one of embodiments 1-43, wherein the polymer composition has a gelation time of about 1 minute to about 4 minutes at a temperature of about 37° C.

Embodiment 45 is the polymer composition of any one of embodiments 1-44, wherein the polymer composition has a gelation time of about 2 minutes to about 8 minutes at a temperature of about 37° C.

Embodiment 46 is the polymer composition of any one of embodiments 1-45, wherein the gel, when formed in the middle ear, has a residence time of at least 1 week.

Embodiment 47 is the polymer composition of any one of embodiments 1-46, wherein the gel, when formed in the middle ear, has a residence time of at least 2 weeks.

Embodiment 48 is the polymer composition of any one of embodiments 1-47, wherein the gel, when formed in the middle ear, has a residence time of at least 1 month.

Embodiment 49 is the polymer composition of any one of embodiments 1-48, wherein the gel, when formed in the middle ear, has a residence time of at least 2 months.

Embodiment 50 is the polymer composition of any one of embodiments 1-49, wherein the gel has a resorption time of about 5 days to about 30 days at 50° C. in PBS.

Embodiment 51 is the polymer composition of any one of embodiments 1-50, wherein the gel has a resorption time of about 7 days to about 15 days at 50° C. in PBS.

Embodiment 52 is the polymer composition of any one of embodiments 1-51, wherein the polymer composition has a pH of about 6.4 to about 7.4.

Embodiment 53 is the polymer composition of any one of embodiments 1-52, wherein the polymer composition has a pH of about 6.0 and 7.0.

Embodiment 54 is the polymer composition of any one of embodiments 1-53, wherein the polymer composition has a pH of about 5.5 to about 8.0.

Embodiment 55 is the polymer composition of any one of embodiments 1-54, wherein the gel, following equilibration in phosphate-buffered saline (PBS) for 1 days, swells less than 80%.

Embodiment 56 is the polymer composition of any one of embodiments 1-55, wherein the gel, following equilibration in phosphate-buffered saline (PBS) for 1 days, swells less than 60%.

Embodiment 57 is the polymer composition of any one of embodiments 1-56, wherein the polymer composition has a viscosity of about 1 mPa·s to about 100 mPa·s.

Embodiment 58 is the polymer composition of any one of embodiments 1-57, wherein the polymer composition has a viscosity of about 1 mPa·s to about 50 mPa·s.

Embodiment 59 is the polymer composition of any one of embodiments 1-58, wherein the gel is hypotonic to the endolymph or perilymph.

Embodiment 60 is the polymer composition of any one of embodiments 1-58, wherein the gel is isotonic to the endolymph or perilymph.

Embodiment 61 is the polymer composition of any one of embodiments 1-58, wherein the gel is hypertonic to the endolymph or perilymph.

Embodiment 62 is the polymer composition of any one of embodiments 1-61, wherein the gel has an osmolality of about 300 mOsmol/kg to about 600 mOsmol/kg.

Embodiment 63 is the polymer composition of any one of embodiments 1-62, wherein the gel has a pH of about 6.0 to about 7.7.

Embodiment 64 is the polymer composition of any one of embodiments 1-63, wherein the gel has a pH of about 6.6 to about 6.8.

Embodiment 65 is the polymer composition of any one of embodiments 1-64, wherein the gel has a pH of about 6.0 to about 6.5.

Embodiment 66 is the polymer composition of any one of embodiments 1-9 or 21-65, wherein the ratio of the first functional group to the second functional group is about 0.8:1.2 to about 1.2:0.8.

Embodiment 67 is the polymer composition of any one of embodiments 1-66, wherein the ratio of the first functional group to the second functional group is about 0.9:1 to about 1:0.9.

Embodiment 68 is the polymer composition of any one of embodiments 1-67, wherein the ratio of the first functional group to the second functional group is about 1:1.

Embodiment 69 is the polymer composition of any one of embodiments 1-68, wherein the functional polymer is a modified PEG.

Embodiment 70 is the polymer composition of any one of embodiments 1-69, wherein the first functional group comprises an electrophile and the second functional group comprises a nucleophile.

Embodiment 71 is the polymer composition of any one of embodiments 1-70, wherein the first functional group comprises a succinimidyl ester.

Embodiment 72 is the polymer composition of any one of embodiments 1-70, wherein the functional group is selected from the group consisting of a succinimidyl succinate, a succinimidyl glutarate, a succinimidyl adipate, a succinimidyl gluraramide, a succinimidyl carbonate, a succinimidyl carboxymethyl ester, or a combination thereof.

Embodiment 73 is the polymer composition of any one of embodiments 1-72, wherein the second functional group comprises a primary amine.

Embodiment 74 is the polymer composition of any one of embodiments 1-73, wherein the functional polymer is pentaerythritol poly(ethylene glycol) ether tetrasuccinimidyl glutarate.

Embodiment 75 is the polymer composition of any one of embodiments 1-74, wherein the crosslinker comprises polylysine, or a salt thereof.

Embodiment 76 is the polymer composition of any one of embodiments 1-75, wherein the crosslinker comprises trilysine, or a salt thereof.

Embodiment 77 is the polymer composition of any one of embodiments 1-76, wherein the first functional group comprises a nucleophile and the second functional group comprises an electrophile.

Embodiment 78 is the polymer composition of embodiment 77, wherein the first functional group comprises a primary amine.

Embodiment 79 is the polymer composition of embodiment 77 or embodiment 78, wherein the second functional group comprises a succinimidyl ester.

Embodiment 80 is an extended release otic composition comprising:
the polymer composition of any one of embodiments 1-79; and
an active agent.

Embodiment 81 is the extended release otic composition of embodiment 80, wherein the active agent is selected from the group consisting of a therapeutic agent, a prophylactic agent, a diagnostic or visualization agent, and combinations thereof.

Embodiment 82 is the extended release otic composition of embodiment 81, wherein the therapeutic agent or prophylactic agent is selected from the group consisting of a protein, a carbohydrate, a nucleic acid, a small molecule, and combinations thereof.

Embodiment 83 is the extended release otic composition of embodiment 82, wherein the protein is selected from the group consisting of an enzyme, a growth factor, an antibody or an antigen-binding fragment thereof, and combinations thereof.

Embodiment 84 is the extended release otic composition of embodiment 82 or embodiment 83, wherein the carbohydrate is a glycosaminoglycan.

Embodiment 85 is the extended release otic composition of any one of embodiments 82-84, wherein the nucleic acid is selected from the group consisting of an antisense oligonucleotide, an aptamer, a micro RNA, a short interfering RNA, a ribozyme, and combinations thereof.

Embodiment 86 is the extended release otic composition of any one of embodiments 82-85, wherein the small molecule is selected from the group consisting of an antibiotic, an antineoplastic agent, a local anesthetic, a steroid, a hormone, an anti-apoptotic agent, an angiogenic agent, an anti-angiogenic agent, a neurotransmitter, a psychoactive drug, an anti-inflammatory, and combinations thereof.

Embodiment 84 is the extended release otic composition of any one of embodiments 82-86, wherein the small molecule is an inhibitor of Apaf-1.

Embodiment 88 is the extended release otic composition of any one of embodiments 82-84, wherein the active agent is a tyrosine kinase inhibitor.

Embodiment 89 is the extended release otic composition of any one of embodiments 80-88, wherein the active agent is a VEGF inhibitor.

Embodiment 90 is the extended release otic composition of embodiment 89, wherein the VEGF inhibitor is selected from the group consisting of agerafenib, altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

Embodiment 91 is the extended release otic composition of embodiment 89 or embodiment 90, wherein the VEGF inhibitor comprises an antibody or antigen-binding fragment thereof.

Embodiment 92 is the extended release otic composition of embodiment 91, wherein the antibody or antigen-binding fragment thereof is selected from the group consisting of alacizumab, bevacizumab, icrucumab, ramucirumab, ranibizumab, and combinations thereof.

Embodiment 93 is the extended release otic composition of any one of embodiments 89-92, wherein the VEGF inhibitor comprises a decoy receptor.

Embodiment 94 is the extended release otic composition of embodiment 92, wherein the decoy receptor is aflibercept.

Embodiment 95 is the extended release otic composition of any one of embodiments 89-94, wherein the VEGF inhibitor comprises an allosteric modulator of a VEGFR.

Embodiment 96 is the extended release otic composition of embodiment 95, wherein the allosteric modulator is cyclotraxin B.

Embodiment 97 is the extended release otic composition of any one of embodiments 89-96, wherein the VEGF inhibitor is at least 10-fold selective for VEGFR2 over another VEGFR.

Embodiment 98 is the extended release otic composition of any one of embodiments 89-97, wherein the VEGF inhibitor is at least 20-fold selective for VEGFR2 over another VEGFR.

Embodiment 99 is the extended release otic composition of any one of embodiments 89-98, wherein the VEGF inhibitor is at least 50-fold selective for VEGFR2 over another VEGFR.

Embodiment 100 is the extended release otic composition of any one of embodiments 88-99, wherein the tyrosine kinase inhibitor or VEGF inhibitor is present in an amount sufficient to reduce edema and lymphatic dysfunction in an affected ear.

Embodiment 101 is the extended release otic composition of any one of embodiments 82-100, wherein the active agent comprises an anti-inflammatory.

Embodiment 102 is the extended release otic composition of any one of embodiments 82-101, wherein the active agent comprises a steroid.

Embodiment 103 is the extended release otic composition of embodiment 102, wherein the active agent comprises a glucocorticoid.

Embodiment 104 is the extended release otic composition of embodiment 103, wherein the active agent comprises dexamethasone.

Embodiment 105 is the extended release otic composition of any one of embodiments 80-101, wherein the active agent does not comprise a steroid.

Embodiment 106 is the extended release otic composition of any one of embodiments 80-105, wherein the active agent comprises a diagnostic or visualization agent.

Embodiment 107 is the extended release otic composition of embodiment 106, wherein the diagnostic or visualization agent is selected from the group consisting of a dye, a fluorophore, an MRI contrast agent, and combinations thereof.

Embodiment 108 is the extended release otic composition of any one of embodiments 80-107, wherein the active agent is present in the extended release otic composition in the form of microparticles.

Embodiment 109 is the extended release otic composition of any one of embodiments 80-108, wherein the active agent is present in the extended release otic composition in the form of nanoparticles.

Embodiment 110 is the extended release otic composition of any one of embodiments 80-109, wherein the active agent is present in an amount of about 0.01% to about 40% by weight of the polymer composition.

Embodiment 111 is the extended release otic composition of any one of embodiments 80-110, wherein the active agent is present in an amount of about 0.1% to about 20% by weight of the polymer composition.

Embodiment 112 is the extended release otic composition of any one of embodiments 80-111, wherein the active agent is present in an amount of about 1% to about 15% by weight of the polymer composition.

Embodiment 113 is the extended release otic composition of any one of embodiments 80-112, wherein the extended release otic composition has a cumulative release of the active agent of about 30% to about 50% at about 3 weeks, when equilibrated against an excess of PBS at 37° C.

Embodiment 114 is the extended release otic composition of any one of embodiments 80-113, further comprising an excipient.

Embodiment 115 is the extended release otic composition of embodiment 114, wherein the excipient is selected from the group consisting of a buffer, a tonicity agent, a mucoadhesive agent, a stabilizing agent, a preservative, a carriers, a penetration enhancer, a diluent, a dispersing agent, a viscosity modifying agent, a solubilizer, an osmolarity modifying agent, and combinations thereof.

Embodiment 116 is an extended release otic composition comprising:
about 5% to about 15% of pentaerythritol poly(ethylene glycol) ether tetrasuccinimidyl glutarate;
about 0.05% to about 0.6% by weight of trilysine or a salt thereof,
about 0.01% to about 40% by weight of dexamethasone; and
water.

Embodiment 117 is an extended release otic composition comprising:
about 7% to about 9% of pentaerythritol poly(ethylene glycol) ether tetrasuccinimidyl glutarate;
about 0.1% to about 0.3% by weight of trilysine;
about 1% to about 10% by weight of dexamethasone; and
water.

Embodiment 118 is an extended release otic composition comprising:
about 8.3% of pentaerythritol poly(ethylene glycol) ether tetrasuccinimidyl glutarate;
about 0.2% by weight of trilysine or a salt thereof,
about 6% by weight of dexamethasone; and
water.

Embodiment 119 is the extended release otic composition of any one of embodiments 116-118, further comprising:
about 0.01% to about 3.0% by weight of sodium borate decahydrate;
about 0.01% to about 3.0% by weight of sodium phosphate;
about 0.01% to about 3.0% by weight of phosphoric acid;
about 0% to about 0.5% of FD&C Blue #1; and
about 0% to about 0.01% by weight of butylated hydroxytoluene.

Embodiment 120 is the extended release otic composition of any one of embodiments 116-118, further comprising:
about 0.05% to about 2.0% by weight of sodium borate decahydrate;
about 0.05% to about 2.0% by weight of sodium phosphate;
about 0.05% to about 2.0% by weight of phosphoric acid;
about 0% to about 0.05% of FD&C Blue #1; and
about 0% to about 0.005% by weight of butylated hydroxytoluene.

Embodiment 121 is the extended release otic composition of any one of embodiments 116-118, further comprising:
about 1.2% by weight of sodium borate decahydrate;
about 1.1% to about 3.0% by weight of sodium phosphate;
about 0.9% to about 3.0% by weight of phosphoric acid;
about 0.01% of FD&C Blue #1; and
about 0.002% by weight of butylated hydroxytoluene.

Embodiment 122 is the extended release otic composition of any one of embodiments 116-118, further comprising:
about 0.01% to about 6.0% by weight of sodium phosphate;
about 0% to about 0.5% of FD&C Blue #1; and
about 0% to about 0.01% by weight of butylated hydroxytoluene.

Embodiment 123 is the extended release otic composition of any one of embodiments 116-118, further comprising:
about 0.05% to about 6.0% by weight of sodium phosphate;
about 0% to about 0.05% of FD&C Blue #1; and
about 0% to about 0.005% by weight of butylated hydroxytoluene.

Embodiment 124 is the extended release otic composition of any one of embodiments 116-118, further comprising:
about 2.0% to about 6.0% by weight of sodium phosphate;
about 0.01% of FD&C Blue #1; and
about 0.002% by weight of butylated hydroxytoluene.

Embodiment 125 is a gel formed by the polymer composition of any one of embodiments 1-79 or the extended release otic composition of any one of embodiments 80-124.

Embodiment 126 is manufacture of a medicament comprising the extended release otic composition of any one of embodiments 80-124 for the treatment of an otic disease or disorder.

Embodiment 127 is a method of preparing an extended release otic composition, the method comprising:
combining a solution or suspension of a functional polymer, wherein the functional polymer comprises a first functional group, a solution or suspension of a crosslinker, wherein the crosslinker comprises a second functional group, and an active agent to form an extended release otic composition, such that the functional polymer is present in an amount of about 5% to about 15% by weight of the extended release otic composition and the crosslinker is present in the extended release otic composition in an amount of about 0.05% to about 0.6% by weight of the extended release otic composition, and wherein a crosslinking reaction can occur between the first functional group and the second functional group to form a gel.

Embodiment 128 is a method of preparing an extended release otic composition, the method comprising:
(a) making a solution or suspension of a functional polymer, wherein the functional polymer comprises a first functional group;
(b) making a solution or suspension of a crosslinker, wherein the crosslinker comprises a second functional group; and
(c) combining the solution or suspension of the functional polymer and the solution or suspension of the crosslinker to form an extended release otic composition, such that the functional polymer is present in an amount of about 5% to about 15% by weight of the extended release otic composition and the crosslinker is present in the extended release otic composition in an amount of about 0.05% to about 0.6% by weight of the extended release otic composition, wherein a crosslinking reaction can occur between the first functional group and the second functional group to form a gel.

Embodiment 129 is a method of preparing an extended release otic composition, the method comprising:
(a) making a solution or suspension of a functional polymer, wherein the functional polymer comprises a first functional group; and
(b) combining the solution or suspension of the functional polymer with a solution or suspension of a crosslinker, wherein the crosslinker comprises a second functional group, such that the functional polymer is present in an amount of about 5% to about 15% by weight of the extended release otic composition and the crosslinker is present in the extended release otic composition in an amount of about 0.05% to about 0.6% by weight of the extended release otic composition,
wherein a crosslinking reaction can occur between the first functional group and the second functional group to form a gel.

Embodiment 130 is a method of preparing an extended release otic composition, the method comprising:
(a) making a solution or suspension of a functional polymer, wherein the functional polymer comprises a first functional group;
(b) altering the pH of a solution or suspension of a crosslinker, wherein the crosslinker comprises a second functional group; and
(c) combining the solution or suspension of the functional polymer with the solution or suspension of the crosslinker,
wherein a crosslinking reaction can occur between the first functional group and the second functional group to form a gel.

Embodiment 131 is a method of preparing an extended release otic composition, the method comprising:
(a) making a solution or suspension of a crosslinker, wherein the crosslinker comprises a second functional group; and
(b) combining the solution or suspension of the crosslinker with a solution or suspension of a functional polymer, wherein the functional polymer comprises a first functional group, such that the functional polymer is present in an amount of about 5% to about 15% by weight of the extended release otic composition and the crosslinker is present in the extended release otic composition in an amount of about 0.05% to about 0.6% by weight of the extended release otic composition,
wherein a crosslinking reaction can occur between the first functional group and the second functional group to form a gel.

Embodiment 132 is a method of preparing an extended release otic composition, the method comprising:
(a) making a solution or suspension of a crosslinker, wherein the crosslinker comprises a second functional group;
(b) altering the pH of a solution or suspension of a functional polymer, wherein the functional polymer comprises a first functional group; and
(c) combining the solution or suspension of the functional polymer with the solution or suspension of the crosslinker,
wherein a crosslinking reaction can occur between the first functional group and the second functional group to form a gel.

Embodiment 133 is the method of any one of embodiments 127-132, wherein an active agent is present in the solution or suspension of the functional polymer.

Embodiment 134 is the method of embodiment 133, wherein the active agent is combined with the functional polymer prior to making the solution or suspension of the functional polymer.

Embodiment 135 is the method of embodiment 133, wherein the active agent is combined with the solution or suspension of the functional polymer.

Embodiment 136 is the method of any one of embodiments 127-135, wherein combining the solution or suspension of the functional polymer with the solution or suspension of the crosslinker comprises combining the solution or suspension of the functional polymer and the solution or suspension of the crosslinker, with an active agent.

Embodiment 137 is the method of embodiment 136, wherein the active agent is provided as a solid.

Embodiment 138 is the method of embodiment 136, wherein the active agent is provided as a solution or suspension.

Embodiment 139 is the method of any one of embodiments 127-138, wherein the extended release otic composition is the extended release otic composition of any one of embodiments 80-124.

Embodiment 140 is a method of treating an otic disease or disorder in a subject, the method comprising:
identifying a subject as having an otic disease or disorder; and
administering a therapeutically effective amount of the extended release otic composition of any one of embodiments 80-124 to an affected ear of the subject.

Embodiment 141 is a method of treating an otic disease or disorder in a subject in need thereof, the method comprising:
administering a therapeutically effective amount of the extended release otic composition of any one of embodiments 80-124 to an ear of the subject.

Embodiment 142 is a method of treating an otic disease or disorder in a subject, the method comprising:
(i) preparing an extended release otic composition by the method of any one of embodiments 127-139; and
(ii) administering a therapeutically effective amount of the extended release otic composition to an ear of a subject in need thereof.

Embodiment 143 is a method of treating an otic disease or disorder in a subject, the method comprising:
(i) identifying a subject as having an otic disease or disorder;
(ii) preparing an extended release otic composition by the method of any one of embodiments 127-139; and
(iii) administering a therapeutically effective amount of the extended release otic composition to an affected ear of the subject.

Embodiment 144 is the method of any one of embodiments 140-125, wherein the otic disease or disorder is selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.

Embodiment 145 is the method of embodiment 126, wherein the sensorineural hearing loss is sudden sensorineural hearing loss.

Embodiment 146 is the method of embodiment 126, wherein the sensorineural hearing loss is associated with diabetes.

Embodiment 147 is a method of treating Ménière's Disease in a subject, the method comprising:
administering a therapeutically effective amount of the extended release otic composition of any one of embodiments 80-124 to an ear of a subject in need thereof.

Embodiment 148 is a method of treating Ménière's Disease in a subject, the method comprising:
(i) identifying a subject as having Ménière's Disease;
(ii) administering a therapeutically effective amount of the extended release otic composition of any one of embodiments 80-124 to an affected ear of the subject.

Embodiment 149 is a method of treating Ménière's Disease in a subject, the method comprising:
(i) preparing an extended release otic composition by the method of any one of embodiments 127-139; and
(ii) administering a therapeutically effective amount of the extended release otic composition to an ear of a subject in need thereof.

Embodiment 150 is a method of treating Ménière's Disease in a subject, the method comprising:
(i) identifying a subject as having Ménière's Disease;
(ii) preparing an extended release otic composition by the method of any one of embodiments 127-139; and
(iii) administering a therapeutically effective amount of the extended release otic composition to an affected ear of the subject.

Embodiment 151 is the method of any one of embodiments 140-150, wherein the administering occurs less than 10 minutes after combining the solution or suspension of the functional polymer with the solution or suspension of the crosslinker.

Embodiment 152 is the method of any one of embodiments 140-151, wherein the administering occurs less than 5 minutes after combining the solution or suspension of the functional polymer with the solution or suspension of the crosslinker.

Embodiment 153 is the method of any one of embodiments 140-152, wherein the administering comprises administering about 5 μL to about 500 μL of the extended release otic composition.

Embodiment 154 is the method of any one of embodiments 140-153, wherein the administering comprises administering about 50 μL to about 200 μL of the extended release otic composition.

Embodiment 155 is the method of any one of embodiments 140-153, wherein the administering comprises administering about 40 μL to about 60 μL of the extended release otic composition.

Embodiment 156 is the method of any one of embodiments 140-153, wherein the administering comprises administering about 50 μL of the extended release otic composition.

Embodiment 157 is the method of any one of embodiments 140-156, wherein the administering comprises administering such that the extended release otic composition is in contact with the round window membrane.

Embodiment 158 is the method of any one of embodiments 140-157, wherein the administering comprises administering such that the extended release otic composition does not engulf any of the ossicles.

Embodiment 159 is the method of any one of embodiments 140-158, wherein the administering comprises administering such that the extended release otic composition does not contact any of the ossicles.

Embodiment 160 is the method of any one of embodiments 140-159, wherein the extended release otic composition is a liquid during the administering.

Embodiment 161 is the method of any one of embodiments 140-160, wherein the extended release otic composition is not exposed to a temperature above about 26° C. before the administering.

Embodiment 162 is the method of any one of embodiments 140-161, wherein the extended release otic composition has a temperature of about 20° C. to about 25° C. during the administering.

Embodiment 163 is the method of any one of embodiments 140-162, wherein the administering comprises injecting through the tympanic membrane.

Embodiment 164 is a method of treating an otic disease or disorder in a subject, the method comprising:
identifying a subject as having an otic disease or disorder; and
administering a therapeutically effective amount of tyrosine kinase inhibitor to the subject.

Embodiment 165 is the method of embodiment 164, wherein the otic disease or disorder is selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.

Embodiment 166 is the method of embodiment 165, wherein the sensorineural hearing loss is sudden sensorineural hearing loss.

Embodiment 167 is the method of embodiment 165, wherein the sensorineural hearing loss is associated with diabetes.

Embodiment 168 is a method of treating Ménière's Disease in a subject, the method comprising:
(i) identifying a subject as having Ménière's Disease; and
(ii) administering a therapeutically effective amount of tyrosine kinase inhibitor to the subject.

Embodiment 169 is a method of treating Ménière's Disease in a subject, the method comprising:
administering a therapeutically effective amount of tyrosine kinase inhibitor to a subject in need thereof.

Embodiment 170 is the method of any one of embodiments 168-169, wherein the administering comprises systemic administration.

Embodiment 171 is the method of any one of embodiments 168-169, wherein the administering comprises administering to an affected ear of the subject.

Embodiment 172 is the method of any one of embodiments 168-171, wherein the tyrosine kinase inhibitor comprises a VEGF inhibitor.

Embodiment 173 is the method of embodiment 172, wherein the VEGF inhibitor is selected from the group consisting of agerafenib, altiratinib, apatinib, axitinib, cabozantinib, cediranib, lapatinib, lenvatinib, motesanib, nintedanib, pazopanib, pegaptanib, rebastinib, regorafenib, semaxanib, sorafenib, sunitinib, toceranib, tivozanib, vandetanib, and combinations thereof.

Embodiment 174 is the method of embodiment 172 or embodiment 173, wherein the VEGF inhibitor comprises an antibody or antigen-binding fragment thereof.

Embodiment 175 is the method of embodiment 174, wherein the antibody or antigen-binding fragment thereof is selected from the group consisting of alacizumab, bevacizumab, icrucumab, ramucirumab, ranibizumab, and combinations thereof.

Embodiment 176 is the method of any one of embodiments 172-175, wherein the VEGF inhibitor comprises a decoy receptor.

Embodiment 177 is the method of embodiment 176, wherein the decoy receptor is aflibercept.

Embodiment 178 is the method of any one of embodiments 172-177, wherein the VEGF inhibitor comprises an allosteric modulator of a VEGFR.

Embodiment 179 is the method of embodiment 178, wherein the allosteric modulator of a VEGFR is cyclotraxin B.

Embodiment 180 is the method of any one of embodiments 172-179, wherein the VEGF inhibitor is at least 10-fold selective for VEGFR2 over another VEGFR.

Embodiment 181 is the method of any one of embodiments 172-180, wherein the VEGF inhibitor is at least 20-fold selective for VEGFR2 over another VEGFR.

Embodiment 182 is the method of any one of embodiments 172-181, wherein the VEGF inhibitor is at least 50-fold selective for VEGFR2 over another VEGFR.

Embodiment 183 is the method of any one of embodiments 168-182, wherein the amount of the tyrosine kinase inhibitor is sufficient to reduce edema and lymphatic dysfunction in an affected ear.

Embodiment 184 is the method of any one of embodiments 168-169 or 171-183, wherein the tyrosine kinase inhibitor is provided in the form of the extended release otic composition of any one of embodiments 80-124.

The present invention will be further understood by reference to the following non-limiting examples.

### EXAMPLES

### Example 1: Impact of pH on Gelation Time of PEG-Trilysine Polymer

### Materials and Methods

A commercial PEG-trilysine polymer system (COVIDIEN Ref DS-D-5005) forms a gel in approximately 3 seconds after mixing equal volumes of a trilysine amine solution and a PEG ester solution. In order to slow down the crosslinking reaction to enable time for injection into the middle ear, the pH of the trilysine solution was modified by addition of HCl (1 N HCl, Millipore) as described in Table 1. The PEG ester solution was prepared per package insert instructions and used within an hour of reconstitution. Gelation time was measured at room temperature by monitoring time for stir bar rotation to cease.

### Results

As shown in Table 1, adjusting the PEG-trilysine polymer trilysine solution from pH 10 to pH 8.4 was successful in slowing gelation time from 3 seconds to about 1 minute. In earlier screening studies, samples prepared with trilysine solution adjusted further to pH 6.8 did not form a gel after 25 minutes of observation.

TABLE 1
Gelation time and degradation of pH adjusted PEG-trilysine polymer

| | | | | |
|---|---|---|---|---|
| pH of modified trilysine solution | 9.9 | 9.1 | 8.7 | 8.4 |
| Volume (mL) of 1N HCl added | 0 | 0.10 | 0.14 | 0.17 |
| per mL of trilysine solution | | | | |
| Percentage of original trilysine | 100% | 91% | 88% | 85% |
| solution in modified trilysine | | | | |
| solution | | | | |
| Gelation time (seconds), n = 2 | 3, 3 | 15, 15 | 26, 25 | 68, 55 |
| Days at room temperature when | 1.5, 1.5 | 11.5, 12.5 | 47, 47 | >100, |
| gel sample was found liquid, | | | | >100 |
| n = 2 | | | | |

### Example 2: Impact of pH on PEG-Trilysine Polymer Gel Duration

### Materials and Methods

Vials from the gelation time measurements in Example 1 were monitored to observe when the gel degraded and became a liquid. The vials with stir bars were stored inverted at room temperature and the times when the samples were found liquid were recorded as shown in Table 1.

### Results

Unmodified PEG-trilysine polymer (i.e., trilysine pH 10) was liquid after 1.5 days of storage at room temperature sealed in a glass vial whereas samples prepared with trilysine adjusted to pH 8.4 were still gel after more than three months.

In addition to affecting the crosslink formation reaction, pH affects the hydrolysis degradation reaction. Preferred formulations for injection to the middle ear have trilysine solution pH between 6 and 8.4, more preferably between 6.5 and 8. These pH values are also favorable from a tissue biocompatibility perspective.

### Example 3: Impact of Drug Particle Concentration on Gelation Time

### Materials and Methods

PEG-trilysine polymer trilysine solution was pH adjusted with 1 N HCl as described in Example 1. Micronized dexamethasone (Dex; Spectrum Chemical DE121) was then added to the pH-adjusted trilysine solution and vortexed. One hundred microliters of the pH-adjusted trilysine drug suspension was then added to 100 microliters of the PEG-trilysine polymer PEG solution in a glass vial for stir bar test of gelation time. The time post addition when the stir bar ceased rotation was recorded.

### Results

While addition of HCl to the trilysine polymer solution dilutes the trilysine and yields less than 1:1 stoichiometry when added to PEG-trilysine polymer PEG solution, the addition of drug particles further dilutes and reduces the stoichiometry.

TABLE 2
Gelation time of pH adjusted trilysine containing up to 6 wt %
dexamethasone

| | | | | |
|---|---|---|---|---|
| Dexamethasone wt % in final gel | 0 | 1 | 2 | 6 |
| Dexamethasone wt % in trilysine solution | 0 | 2 | 4 | 12 |
| pH of modified trilysine solution | 8.4 | 8.4 | 8.4 | 8.4 |
| Percentage of original trilysine solution in pH | 85% | 84% | 80% | 75% |
| modified trilysine drug suspension | | | | |
| Gelation time (seconds) | 48 | 52 | 52 | 55 |

As shown in Table 2, the fraction of original trilysine solution in pH 8.4 modified trilysine contains 85% of the original trilysine solution and addition of 12 wt % dexamethasone lowers the amount of original trilysine solution to 75%.

As also shown in Table 2, adding up to 12 wt/% dexamethasone to the pH 8.4 trilysine solution did not have a substantial impact on the gelation time measured by the stir bar test.

### Example 4: Drug Release and Erosion Testing for 0 to 6 wt % Dexamethasone in pH Modified PEG-Trilysine Polymer

### Materials and Methods

In vitro testing of drug release and gel erosion was performed using 200 μL of gel sample in inserts (CORNING TRANSWELL® 3414 inserts with 0.4 μm pore polycarbonate membranes). The inserts were submerged in 45 mL of receptor fluid (pH 7.4 PBS, SIGMA-ALDRICH® P5368) in an incubator at 37° C. to simulate the delivery of drug to potential fluid in the middle ear. The large receptor volume was chosen to keep the drug concentration low (i.e., sink conditions) so that saturation of the receptor solution does not limit the drug release rate. The test measures the maximum drug release rate to PBS for the test configuration: 0.33 cm² area exposed directly to PBS and drug release through membrane with 0.33 cm² area. In addition, swelling and erosion were monitored gravimetrically.

PEG-trilysine polymer samples were prepared in triplicate using pH-adjusted trilysine solution and 0, 1, 3, and 6 wt % dexamethasone as described in Example 3. The two components were combined in a microcentrifuge tube and mixed by vortexing prior to dispensing an aliquot into each insert. At study initiation, samples were submerged in receptor solution and stored at 37° C. Periodically, samples of receptor solution were removed and assayed for drug concentration to determine drug release. At intervals no greater than 9 days, the samples were also moved to new vials of receptor solution. Before sampling or moving inserts, the receptor vials were manually inverted several times to ensure a uniform receptor fluid.

Sample weights were recorded periodically after removing excess fluid with a lab tissue. Percent swelling was calculated as the change in sample weight divided by initial sample weight. Initial sample weights reflect swelling and initial degree of crosslinking.

### Results

As the study proceeded, sample weights were affected by changes due to degradation by hydrolysis. Sample weights first increased as the gel with initial 90% water absorbed water due to contact with receptor fluid. Samples continued to gain weight as the gel was degraded and swelled more due to fewer crosslinks, while gel erosion contributed to reduction in weight. Eventually the gel completely eroded and dissolved into the receptor fluid. As the gel eroded, the remaining solid drug particles tended to settle and flow out of the inserts if the inserts were oriented on their sides and during the manual mixing process before moving inserts to a new vial.

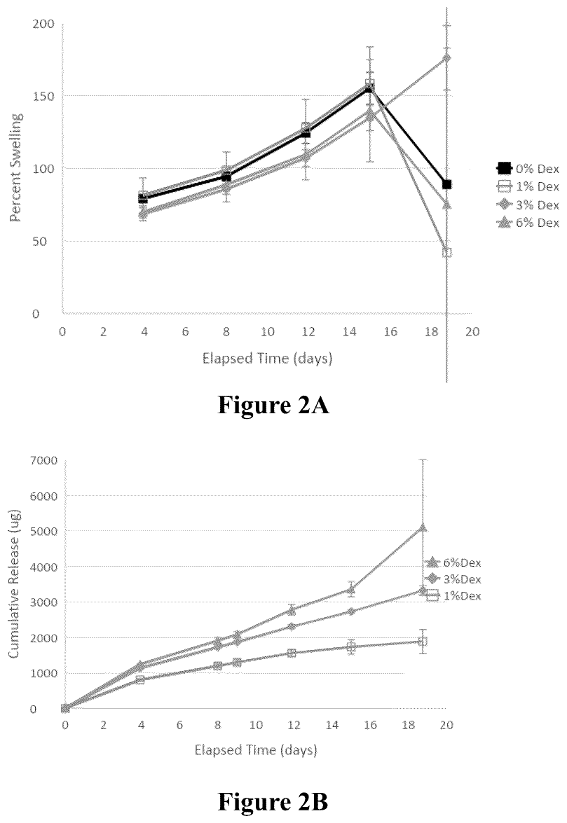

Results in FIG. 2A show samples at Day 4 had initial swelling of 64 to 95% and those values were not strongly dependent on the amount of dexamethasone. All twelve inserts contained gel for at least 15 days. By Day 19, four samples had weight loss from significant erosion. Further evaluation showed the samples with shorter gel duration appeared to be correlated with pot life (time since PEG reconstitution) of the PEG solution rather than dexamethasone content; e.g., samples with longer time since PEG reconstitution have fewer crosslinks and degrade more easily (See, e.g., Example 7).

Drug release from the dexamethasone containing samples is shown in FIG. 2B. The 1% Dex samples were fully depleted of drug by Day 15 while 3% and 6% gels had cumulative release of 48% and 30%, respectively. Once the gel has degraded to a fluid consistency, the remaining drug particles settled and spread out of the insert into the receptor fluid. This resulted in higher amounts of drug released into receptor fluid as demonstrated by the increase in drug release for one of the 6% Dex samples at Day 19.

Observations of clear versus turbid gel were consistent with the measured amounts of drug released. The 1% Dex samples initially had clear gel primarily at the surface exposed directly to PBS and less so at the membrane surface side. Eventually there were no turbid regions of gel in these samples. In the other extreme, only a small amount of clear gel was observed on the PBS exposed surface of the 6% Dex samples.

### Example 5: Tissue Adhesion of 0 to 6 wt % Dexamethasone in pH-Modified PEG-Trilysine Polymer

### Materials and Methods

A qualitative assessment of adhesion to tissue was performed by applying samples to raw turkey breast meat. In parallel with preparation of samples in Example 4, the remaining material was pipetted onto the surface of the turkey meat and a small wooden stick was applied on top. After a few minutes of gelation time, the adhesion was assessed by pulling on the wooden stick.

Testing was performed in triplicate using samples with 0, 1, 3, and 6 wt % final dexamethasone content in PEG-trilysine polymer trilysine adjusted to pH 8.4.

### Results

Better adhesion was found for 3 and 6 wt % Dex samples compared to 0 and 1 wt % Dex samples. In addition, the gels with higher drug content had better cohesion, demonstrating the drug particles can improve gel mechanical properties similar to filler particles that can strengthen viscoelastic materials.

### Example 6: Gelation Time for Higher Dexamethasone Content PEG-Trilysine Polymer

### Materials and Methods

PEG-trilysine polymer samples prepared from 20% Dexamethasone in pH 8.4 adjusted PEG-trilysine polymer trilysine were tested for gelation time by the stir bar test. Sample preparation and testing procedure were similar to that described in Example 1.

### Results

Gelation times were slightly longer for 10% Dex samples than 6% Dex samples as shown by Table 3.

TABLE 3
Gelation time of pH adjusted trilysine containing 6 or 10 wt %
dexamethasone

| | | |
|---|---|---|
| Dexamethasone wt % in final gel | 6 | 10 |
| Dexamethasone wt % in trilysine solution | 12 | 20 |
| pH of modified trilysine solution | 8.4 | 8.4 |
| Percentage of original trilysine solution in pH | 75% | 68% |
| modified trilysine drug suspension | | |
| Gelation time (seconds), n = 3 | 73, 62, 66 | 74, 82, 83 |

The dilution of PEG-trilysine polymer trilysine by addition of 1 N HCl and dexamethasone corresponds to about 68% of the original trilysine in the pH adjusted trilysine drug suspension. The corresponding deviation from 1:1 stoichiometry may be approaching levels insufficient for robust gel formation.

### Example 7: Drug Release and Erosion Testing for 6 wt % Dexamethasone in pH Modified PEG-Trilysine Polymer

### Materials and Methods

In vitro testing of drug release and gel erosion was performed as described in Example 4. Samples were prepared from pH 8.4 adjusted PEG-trilysine polymer trilysine with 12 wt % dexamethasone and the final samples contained 6 wt % dexamethasone. The pH-modified trilysine drug suspension was combined with PEG-trilysine polymer PEG solution using a blending connector with static mixer (Nordson FibriJet® SA-3678). Each blending connector was used to fill six inserts with sample.

### Results

Data for each group of six is designated by the Pot Life of the PEG solution at time of use; i.e., time since PEG was reconstituted.

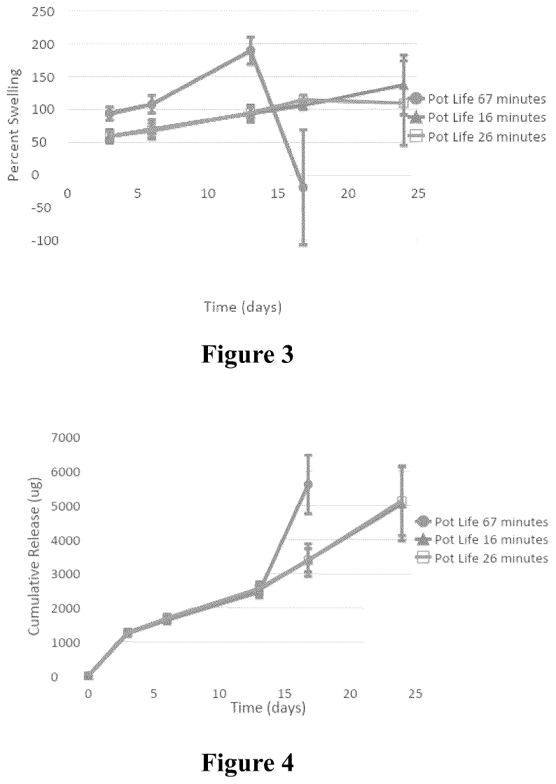

The group with pot life of 67 minutes had higher initial swelling and shorter gel duration than the groups with pot life less than 30 minutes, as shown by FIG. 3. Cumulative drug release, shown by FIG. 4, was very reproducible, irrespective of pot life until the gel was found eroded in the 67 minute pot life group at Day 17. The PEG-trilysine polymer package insert specifies using the PEG solution before the pot life is 60 minutes so that crosslinking occurs before some of the PEG has degraded. These results demonstrate the tests were able to differentiate between samples with varying degrees of crosslinking.

All samples prepared from PEG with pot life less than 30 minutes had gel duration of at least 24 days and were completely eroded at the next time point. These were submerged in PBS receptor solution during storage at 37° C. Aliquots of formulation were stored at 37° C. without PBS in closed microcentrifuge tubes within a moist chamber to avoid dehydration. The two samples prepared with PEG pot life of 30 minutes had gel durations of 60 and more than 80 days when the samples were not in contact with PBS.

### Example 8: In Vitro Comparison of 6 Wt % Dexamethasone in Poloxamer® 407 Thermosensitive Gels Vs. pH Adjusted Peg-Trilysine Polymer Chemically Crosslinked Gels

### Materials and Methods

Larger batches of 12 wt % dexamethasone in pH 8.4 adjusted PEG-trilysine polymer trilysine were prepared by pooling five PEG-trilysine polymer syringes of trilysine solution. The volume of trilysine solution was calculated from the weight of the solution and density of 1.02 g/mL. The volume of trilysine solution was multiplied by 0.08 to calculate the volume of 2 N HCl to be added. The pH of an aliquot was confirmed after addition of acid. Then dexamethasone was added and the vial vortexed to yield a drug suspension with 12 wt % in the pH adjusted trilysine.

The pH-adjusted PEG-trilysine polymer trilysine was combined with PEG solution having pot life no more than 30 minutes. The final formulation had 6 wt % dexamethasone.

A POLOXAMER® 407 gel with 6 wt % dexamethasone was prepared to serve as a comparator. A buffer solution of 0.01 M PBS was prepared from PBS pH 7.4 (Sigma-Aldrich) and Water for Injection (CalbioChem). A 16 wt % POLOXAMER® 407 stock solution was prepared by slowly adding POLOXAMER® 407 (BASF) to cold phosphate buffer while stirring.

Dexamethasone was added to an aliquot of POLOXAMER® 407 stock solution to yield 6 wt % dexamethasone in 15 wt % POLOXAMER® 407; this final formulation is denoted POLOXAMER® 407-Dex. A vial inversion test was performed to confirm POLOXAMER® 407-Dex would gel when placed in an incubator at 30° C.

### Results

### Erosion Visualization of PEG-Trilysine Polymer-Dex Vs POLOXAMER® 407-Dex

Gel erosion was observed in UV disposable microcuvettes filled partially with sample and then filled with PBS. Duplicate cuvettes were filled with 250 μL of sample. In the case of the PEG-trilysine polymer, the two parts were first combined and vortexed in a microcentrifuge tube and transferred to the cuvette. The cuvettes were placed in a 37° C. incubator for 10 minutes for gelation prior to addition of 2 mL of PBS preheated to 37° C. The cuvettes were sealed with parafilm and then stored in the horizontal position at 37° C.

The surface of POLOXAMER® 407-Dex samples began to erode immediately after addition of PBS. As the POLOXAMER® 407 dissolved, the drug particles settled and flowed into lower portions of the cuvette. The POLOXAMER® 407-Dex samples were completely eroded in less than 2 hours.

In contrast, after one day, the appearance of PEG-trilysine polymer-Dex samples prepared in pH modified PEG-trilysine polymer had barely changed. Even at Day 14, the majority of the gel was still intact.

### Drug Release of PEG-Trilysine Polymer-Dex Vs POLOXAMER® 407-Dex

Drug release was measured for 6 wt % dexamethasone in pH 8.4 modified PEG-trilysine polymer (PEG-trilysine polymer-Dex) vs. in POLOXAMER® 407 (407-Dex). Inserts were submerged in 50 mL of PBS receptor fluid as described in Example 4. The two parts of PEG-trilysine polymer-Dex were first combined and vortexed in a microcentrifuge tube and then transferred to the inserts. Samples were placed at 37 C for 45 minutes to form a gel prior to addition of prewarmed PBS.

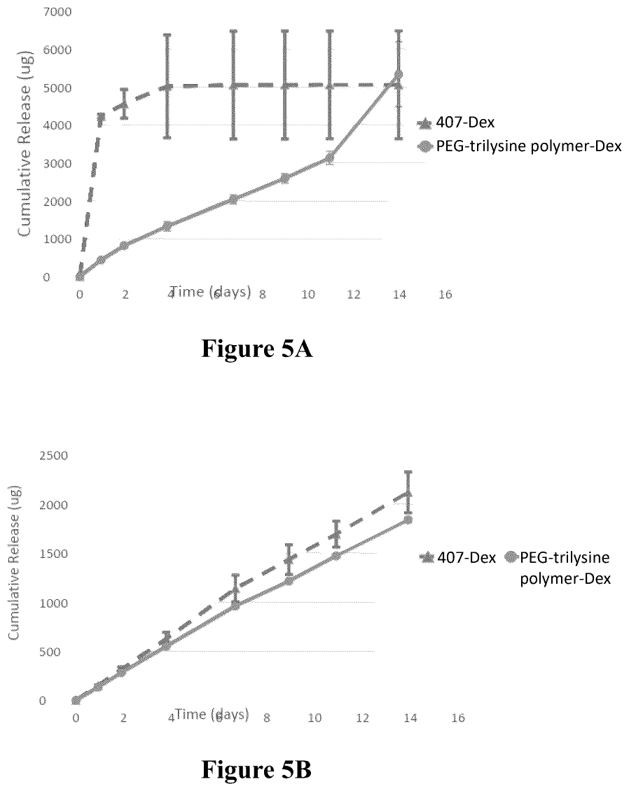

Within the first day, all 407-Dex samples had completely eroded and remaining drug particles had spread out of the insert and into the receptor vial (FIG. 5A). The 50 mL of receptor fluid equilibrated with the drug solid within 1 day and, hence, the amount of drug released was dependent on the volume (50 mL) of receptor fluid. Variable amounts of free solid drug were transferred along with the inserts to the next vial of receptor fluid and eventually the inserts were empty of drug particles.

In contrast, the chemically crosslinked PEG-trilysine polymer-Dex samples delivered drug to the receptor fluid at a constant rate for 11 days. Subsequently, the samples degraded and eroded, with drug particles spreading into the receptor fluid and resulting in faster delivery to receptor fluid.

### Permeation of PEG-Trilysine Polymer-Dex Vs 407-Dex Through a Biomimetic Membrane

In vitro drug permeation of the two formulations was assessed using a biomimetic membrane with a lipid layer between two cellulose layers (PermeaPad Barrier Membrane). The method utilizes equilibrium dialysis cells (Harvard Apparatus) to create a 500 uL PTFE chamber in contact with the membrane. Donor cells that were filled with 480 uL of formulation and heated at 37° C. for 45 minutes for gelation. The cells were then submerged in bottles containing 75 mL of PBS receptor fluid and stored at 37° C. The receptor side of the equilibrium dialysis cells were left open to expose the membrane to the large volume of receptor fluid. Periodically, the receptor fluid was sampled after manual mixing in order to measure the cumulative amount of drug delivered. In this test configuration, all drug delivery occurred by permeation through the biomimetic membrane, different from the previous drug release configuration in inserts which allowed drug to release directly into PBS as well as through the porous membrane of the insert. Drug permeation through the biomimetic membrane is intended to more closely simulate drug permeation through biological tissue such as the round window membrane.

Drug delivery rates of dexamethasone were slightly higher from the poloxamer gels (FIG. 5B). Without being bound by any particular theory, it is believed that the micellar structure of poloxamer gels can help solubilize drugs such as dexamethasone and assist transport from the drug particles to the membrane.

The integrity of the gels during the permeation study was also monitored during the permeation study by looking for appearance of settled drug particles. This evidence of fluid donor was seen at Day 4 and Day 14 for the 407-Dex and PEG-trilysine polymer-Dex samples, respectively. While the permeation setup and nonporous membrane did not allow direct contact with PBS as in the erosion and drug release studies, water was able to cross the biomimetic membrane as suggested by the bulging membrane. This water uptake was sufficient to cause dissolution of the thermosensitive gel by Day 4.

### Gel Duration of Isolated PEG-Trilysine Polymer-Dex Vs 407-Dex at 37° C.

Aliquots of these formulations were stored at 37° C. in closed microcentrifuge tubes within a moist chamber to avoid dehydration. This configuration assesses gel integrity when stored isolated from fluid at body temperature. Both formulations had long gel durations greater than 21 days.

The chemically crosslinked formulation PEG-trilysine polymer-Dex was compared to thermosensitive poloxamer formulation 407-Dex in multiple in vitro tests. The combination of results illustrates the differences expected in erosion and gel integrity when these two formulations are injected into the middle ear. Both are expected to achieve zero order drug delivery across the round window membrane if the formulations remain in contact with the membrane. Poloxamer gels with physical crosslinking will start to erode immediately when in contact with fluid such as mucous or when water is absorbed from contact with tissue. Once the gel becomes fluid, it has the potential to flow and drain away from the round window, leading to short and variable gel duration at the target tissue. On the other hand, formulations chemically crosslinked in situ have longer reproducible retention times at the target tissue and, hence, should provide more durable drug delivery performance.

### Example 9: Dual Role of Crosslinker and Flocculation Agent

A generally preferred characteristic of a drug suspension product is the ability to administer uniform doses. This can be easier to achieve for formulations that do not form a dense sediment during storage and can be easily re-dispersed upon manual agitation of the vial. The settling of drug particles in pH modified trilysine drug suspension and poloxamer formulation described in Example 8 were assessed by visual inspection.

After more than 14 days, the pH-adjusted trilysine drug suspension had a clear top layer of no more than 40% of the volume. This corresponds to a Flocculation Efficiency of >60/12 or 5. Trilysine provided easily dispersible loose flocs of dexamethasone in addition to serving the important role of crosslinking when this component is combined with PEG solution.

Within a few hours, dexamethasone particles in Dex-407 have settled and more than 90% is a clear supernatant. This corresponds to a Flocculation Efficiency of <10/6 or 1.7.

### Example 10: In Vivo Testing of PEG-Trilysine Polymer-Dex Vs 407-Dex

The chemically crosslinked gels prepared from pH-modified PEG-trilysine polymer and thermosensitive gels prepared from Poloxamer 407 (P407) were tested in vivo in a green monkey model. Formulations with 6 wt % dexamethasone were prepared as described in Example 8. The formulations were administered until it was visible at the TM perforation, with final injection volumes ranging from 30 to 100 μL.

Results demonstrating the residence time of these formulations is shown in Table 4A.

TABLE 4A
Persistence of POLOXAMER ® 407 Gel Compared to PEG-trilysine polymer
Gel.

| | | | | Peri- | |
|---|---|---|---|---|---|
| | Animal | | Injected | lymph | |
| Group | ID | Ear | Volume | Sample | Middle Ear Exploration |
| P407 1-day | A843 | Left | 75 uL | Yes | Most gel retained, inside RWM-niche |
| | | Right | 50 uL | Yes | Most gel retained, inside RWM-niche |
| P407 10-day | B192 | Left | 50 uL | Yes | Min gel evidence, not in RWM |
| | | Right | 40 uL | Yes | Min gel evidence, not in RWM |
| P407 22-day | A215 | Left | 60 uL | Yes | No evidence of gel |
| | | Right | 25 uL | Yes | No evidence of gel |
| PEG-trilysine | K090 | Left | 100 uL | Yes | Most gel retained, inside RWM-niche |
| polymer 1-day | | Right | 60 uL | Yes | Most gel retained, inside RWM-niche |
| | A363 | Left | 35 uL | Yes | Most gel retained, inside RWM-niche |
| | | Right | 35 uL | Yes | Most gel retained, inside RWM-niche |
| PEG-trilysine | A071 | Left | 100 uL | Yes | Most gel retained, inside RWM-niche |
| polymer 10- | | Right | 50 uL | Yes | Most gel retained, inside RWM-niche |
| day | B131 | Left | 60 uL | Yes | Most gel retained, inside RWM-niche |
| | | Right | 50 uL | Yes | Most gel retained, inside RWM-niche |
| PEG-trilysine | A592 | Left | 30 uL | Yes | Small amount of gel in RWM-niche |
| polymer 22- | | Right | 50 uL | Yes | No evidence in RWM-niche |
| day | B184 | Left | 50 uL | Yes | No evidence in RWM-niche |
| | | Right | 50 uL | Yes | Opaque gel residue in RWM-niche |

Information on absorption, distribution and pharmacokinetics of the PEG-trilysine polymer formulation by otic administration were obtained from a pharmacokinetic (PK) and tolerability study conducted in the African green monkey (Chlorocebus sabaeus)

The perilymph and plasma concentrations of the PEG-trilysine polymer formulation were evaluated following a single IT injection. Perilymph and plasma levels of test and control articles were measured after 1, 10 and 22 days. Given the small study size (N=9 enrolled) and number of total ears treated, PK data are presented as descriptive statistics only.

Auditory brainstem response thresholds were evaluated before dosing and at termination, and cochlea were collected at necropsy for analysis of cochlear hair cell histology. The ABR test measures whether the animal's cochlea, cochlear nerve, and brainstem responds to each sound stimulus and is often used as a measure of the health of the ear. This same basic test is commonly used to test hearing of newborn humans in hospitals and it is a standard hearing test used in lab animals.

ABRs were performed using a compact auditory electro diagnostic system for stimulus generation and neural response were amplified 100,000 times and band-pass filtered between 300-3,000 hertz (Hz), with an additional 60 Hz notch filter. The amplified and filtered signals were then averaged for 512 artifact-free averages in 10 millisecond (ms) epochs before being plotted on the screen. Initial subjective estimates of threshold were determined at the time of testing. Threshold was defined as the lowest intensity of each stimulus frequency that the subject's brainstem could reliably process.

A total of 9 animals received bilateral intratympanic injections of a formulation in this study, with follow up intervals of 1, 10, and 22 days. Of the 18 treated ears, 12 received the PEG-trilysine polymer formulation and 6 received the P407 formulation.

Table 4B provides a summary of treatment groups and study procedures.

TABLE 4B
Summary of Study Design for 22-Day Safety and Pharmacokinetic Study of the
PEG-trilysine polymer formulation in African Green Monkeys Treatment

| Treatment | Injection | | | Study procedures | | | | |
|---|---|---|---|---|---|---|---|---|
| Group | Volume | | N (ears) | Baseline | Day 0 | Day 1 | Day 10 | Day 22 |
| PEG- | 0.03-0.10 | mL* | 4 | a | x | a, b, c, d | | |
| trilysine | | | 4 | a | x | — | a, b, c, d | |
| polymer | | | 4 | a | x | — | — | a, b, c, d |
| P407 | 0.03-0.10 | mL | 2 | a | x | a, b, c, d | | |

| | | | | | |
|---|---|---|---|---|---|
| 2 | a | x | — | a, b, c, d | |
| 2 | a | x | — | — | a, b, c, d |

*Injection Volume dictated by visual endpoint to ensure filling of the round window niche
Study Procedures:
a = Auditory Brainstem Response (ABR)
b = Plasma Collection for Drug Content
c = Perilymph Collection for Drug Content
d = Cochlear Histology for Hair Cell Counts (one ear of each animal)
x = Intratympanic Injection

Overall, both formulations were well-tolerated, as assessed by electrophysiological hearing measurements, cochlear histology, and behavioral observation. Baseline ABR thresholds were similar to, or better than, those seen in other non-human primate ABR studies. Auditory brain stem response threshold shifts were generally minimal and consistent with threshold increases in both groups in the higher frequencies. In addition, hair cell analysis by cytocochleogram and immunofluorescent staining demonstrated few missing hair cells, at levels consistent with normal aging and unlikely to be related to treatment.

There were no overt changes in subject health and welfare observed during the study. The majority of the PEG-trilysine polymer formulation was retained in the middle ear at Day 10, with residual formulation detected at Day 22. Formulations delivered sustained concentrations of dexamethasone to the perilymph through Day 22, with comparatively low systemic exposure.

The results of this study establish the African green monkey as a model to deliver long-lasting depot formulations of the PEG-trilysine polymer formulation to the inner ear using IT injections. In addition, the study successfully established the African green monkey model to be used in PK and tolerability studies of novel otologic therapeutics.

The absorption and distribution of dexamethasone was also investigated in African green monkeys. Dexamethasone concentrations were evaluated in plasma, perilymph and cochlea.

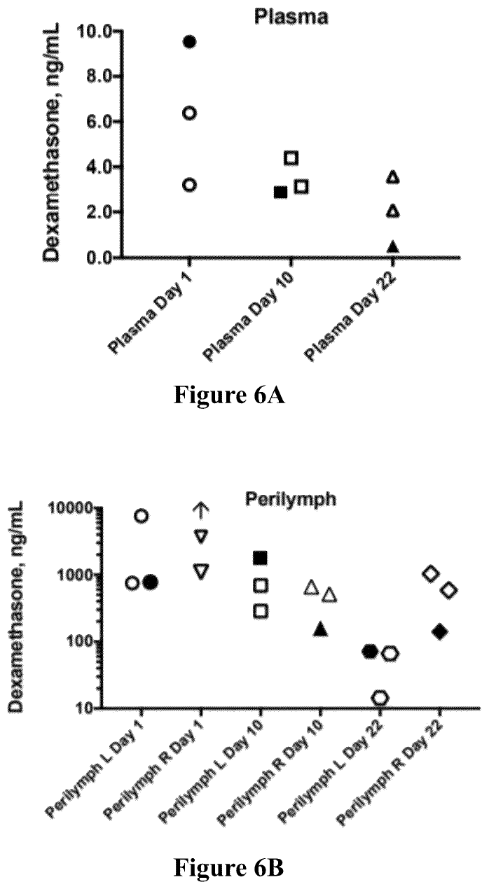

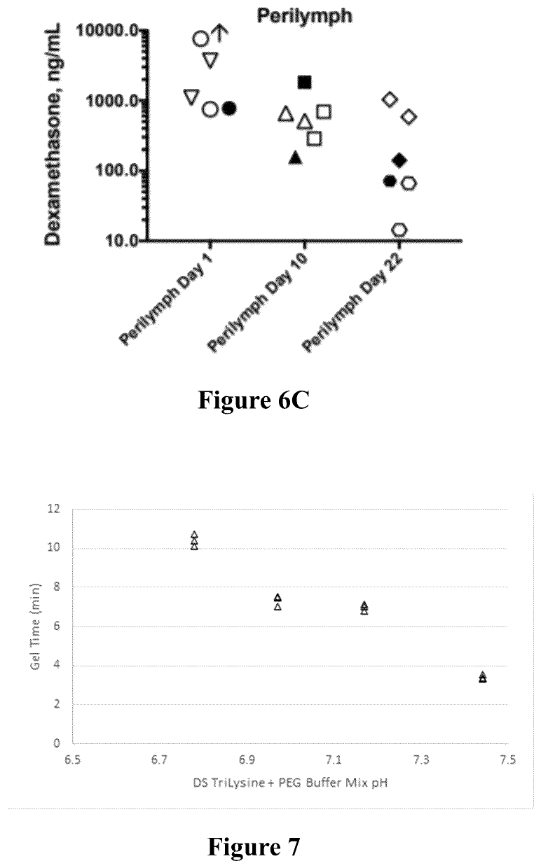

Over time, plasma levels of dexamethasone declined but remained within detectable limits through Day 22, the final timepoint of the study. Levels appeared more sustained for the PEG-trilysine polymer formulation-treated animals, consistent with observations of delayed gel clearance from the middle ear relative to the P407. FIG. 6A shows the total amount of dexamethasone observed in plasma. In FIG. 6A, the vertical axis is concentration of dexamethasone ng/mL. Horizontal axis is time since dosing. Points are individual subjects. Open symbols are from subjects administered the PEG-trilysine polymer formulation and filled symbols are from subjects administered the P407 formulation.

Perilymph dexamethasone concentration remained well above plasma levels throughout the study, consistent with sustained release from the depot formulation. Variability in dexamethasone levels was evident between subjects at each time point (FIGS. 6B and 6C) but demonstrated an overall progressive decrease in perilymph concentrations over the course of the 22-day study. In FIGS. 6B and 6C, the vertical axis is concentration of dexamethasone ng/mL. Horizontal axis is time since dosing. FIG. 6B: Points are individual subjects, for left ears (L, circles, squares, hexagons) and right ears (R, triangles and diamonds). Open symbols are from subjects administered the PEG-trilysine polymer formulation, and filled symbols are from animals administered the P407 formulation. Arrow symbol represents subject A843, administered the P407 formulation, whose dexamethasone level is above the level of detection (limit 9500 ng/mL). FIG. 6C: Same data with left and right ears grouped together. Symbols same as in FIG. 6B.

In the cochlea, variability in dexamethasone levels was evident at each time point. Despite some variation, concentrations over time were also consistent with a slow release from the depot formulation, particularly for the PEG-trilysine polymer formulation.

Sustained exposure of dexamethasone was observed in the targeted perilymph compartment with comparatively lower systemic exposure, as demonstrated by higher dexamethasone levels in perilymph and cochlea than plasma.

Dexamethasone concentration in whole cochlea preparations was typically lower than in perilymph. Variability in dexamethasone levels between ears was evident at each time point. Despite some variation, the group mean concentrations over time was also consistent with a slow release from the depot formulation.

### Example 11: PEG Buffer Composition to Extend Gelation Time

Instead of lowering the pH of the trilysine solution in Example 1 with hydrochloric acid, gelation time was reduced by modifying the composition of the solution used to reconstitute PEG; i.e., PEG diluent.

### Materials and Methods

Diluents were prepared containing 0.13, 0.14, 0.15, and 0.16 M phosphoric acid (Spectrum Chemical) in Water for Injection. 2.6 mL of each diluent was added to a PEG ester powder vial and manually agitated until reconstitution was achieved.

One hundred microliters of PEG ester solution was combined with one hundred microliters of trilysine solution for stir bar testing of gelation time. In addition, pH was measured for equivolume mixtures of the new diluents and trilysine; pH was measured without the presence of PEG ester to avoid formation of a gel during the pH measurement. In order to measure the pH of the final otic composition, one could measure pH of water (e.g., distilled or low impedance) after equilibration with the final otic composition.

### Results

Increasing concentration of phosphoric acid, 0.13 to 0.16 M, in the diluent solutions resulted in decreased pH, 7.44 to 6.78, when mixed in equal volumes with trilysine. As shown in FIG. 7, gelation times between 3 to 11 minutes were achieved by varying the diluent phosphoric acid concentration and resulting mixture pH.

Gelation times of 1 to 10 minutes required for intratympanic injection can be achieved by changing the trilysine or PEG ester solution with components that lower pH.

An additional embodiment for intratympanic injection is envisioned that involves reconstitution of PEG ester powder with a single solution that contains trilysine and all of the pH modifying components

### Example 12: Impact of PEG Functionality on Gelation Time and Gel Duration

Gel properties such as gelation time and gel duration can be impacted by a change in functionality, for example, a reduction from close to 4 NHS per multi-arm PEG to approaching 3 NHS per PEG, The PEG degree of functionality may decrease during storage and in this study was tracked via time since expiration.

### Materials and Methods

Samples were prepared similar to Example 11 except aliquots of PEG were reconstituted with scaled down quantities of diluent based upon weights of the PEG aliquots. Kits were tested with expiration dates ranging from 1.1 years after the testing date to 3.6 years before the testing date. PEG diluents were prepared containing sodium phosphate monobasic (Millipore) and phosphoric acid (Spectrum Chemical) in 0:100, 50:50, and 100:0 ratios in Water for Injection. pH was measured for the diluents alone, PEG ester reconstituted in diluent, and equal volume mixtures of the diluents and trilysine.

Gelation time was measured by stir bar test at room temperature (about 20° C.) by combining one hundred microliters of PEG ester solution with one hundred microliters of trilysine solution. Gel duration was assessed by preparing 0.2 mL of otic composition gel in the bottom of a 2 mL glass vial, adding 1 mL of PBS and storing at 37° C. or 50° C.; samples were inspected by periodically inverting the vials and noting when the gel had become liquid. In addition, the volume of the gel was qualitatively assessed as a measure of crosslinking density.

### Results

The 50:50 diluents were tested with strengths ranging from 0.20 to 0.27 M; the pH of these diluents was about 2.0, increased to about 2.3 for PEG reconstituted into these diluents, and to about 6.5 to 7.1 for diluent added to trilysine solution (no PEG).

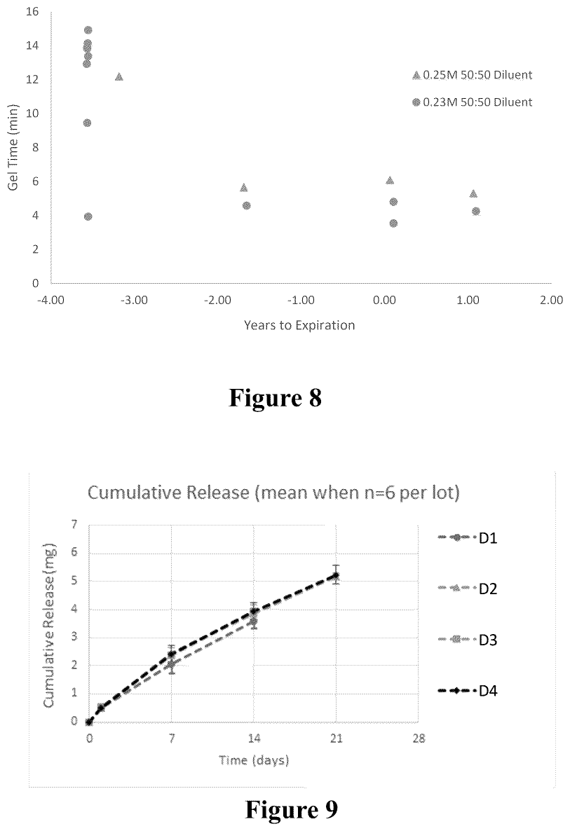

Gelation times of 3.5 to 5 minutes were obtained with 0.23 M 50:50 diluent for kits tested up to 1.7 years post expiration (see FIG. 8). Gelation time increased to about 5 to 6 minutes when the diluent strength was increased to 0.25 M. The majority of the kits tested more than three years post expiration had gelation times of about 12 to 15 minutes, attributed to reduced functionality of the PEG ester in these older kits.

Higher amounts of swelling were observed in the otic compositions prepared from the older kits with longer gelation times, consistent with reduced functionality and lower crosslink density.

Otic compositions prepared with 50:50 diluent had gel durations at the 50° C. accelerated condition of 3 to 4 weeks for the kits with higher functionality PEG and approximately 1 week for the kits with lower functionality PEG. At 37° C., gel durations were 2-3 weeks for the lower PEG functionality compositions; testing is on-going and will have gel durations greater than 5 weeks for the otic compositions prepared with higher functionality PEG.

### Example 13: Impact of Solids Content on Gelation Time

The ability to fine tune gelation time by modification of solids content was explored by addition of water to the gel composition.

### Materials and Methods

Samples were prepared as described in Example 12 from a lot with demonstrated higher functionality. PEG aliquots were reconstituted in diluent containing 50:50 sodium phosphate monobasic (Millipore) and phosphoric acid (Spectrum Chemical) at 0.30 M strength. Otic compositions were prepared by combining equal volumes of Trilysine and PEG solution and optionally additional Water for Injection to increase the final volume by 10% or 20% and hence, decrease the solids content by up to 20%.

Gelation time was measured by stir bar test at room temperature (about 20° C.) by combining one hundred microliters of PEG ester solution with one hundred microliters of trilysine solution and optional additional water. The pH of the final gel was measured by equilibrating 1 mL of distilled water with the gel overnight at room temperature and measuring the pH of the equilibrated solution.

Samples were also prepared in inserts and submerged in PBS at 37° C. for gravimetric assessment of swelling.

### Results

Compared to gelation times of 4.6 and 5.6 minutes for 0.23 and 0.25 M diluent in Example 12, increasing the diluent strength to 0.30 M increased gelation time to 9.5 minutes. Gelation times were further increased to 10.7 and 11.8 minutes by lowering the solids content by 10 and 20%.

The pH of these final otic gel compositions ranged from 6.68 to 6.75. After 2 days submerged in PBS at 37° C., the highest solids content gels had percent swelling of 45%. Reducing solids content by 20% decreased percent swelling to 36%.

### Example 14: Reproducibility of Gel Formation and Swelling

Three polymer compositions (C1-C3) were prepared and evaluated for gel formation parameters and swelling as described in Table 5. Percent swelling was determined gravimetrically using inserts containing 0.20 mL of the composition placed in bottles containing 200 mL of PBS at 37 TC. Gelation ambient temperature was determined using a rotating stir bar test, with gelation identified as the sample coating the stir bar (e.g., the stir bar and gel rotating as a unit), or the stir bar rotating the vessel. Gelation at 37° C. was determined by probing a stir bar with an external magnet on the exterior of a transparent cover; if when probed with the external magnet from above, the stir bar does not exit the sample, then the sample is a gel. Injectability was determined by tester perception of syringe resistance.

TABLE 5

| | C1 | C2 | C3 |
|---|---|---|---|
| PEG-trilysine | A | B | A |
| batch ID | | | |
| Pot life (min) | 2 | 2 | 2 |
| % PEG vs. Target | 100 | 101 | 101 |
| % Buffer vs. | 100 | 101 | 96 |
| Target | | | |
| % Trilysine vs. | 100 | 101 | 102 |
| Target | | | |
| Trilysine/PEG | 100 | 100 | 101 |
| Average Lab | 24.7 | 24.8 | 22.9 |
| Temp (° C.) | | | |
| Injectability at 10 | Yes | Yes | Yes |
| minutes | | | |
| Gelation time | 0.9 | 0.8 | 1.4 |
| (minutes) at 37° C. | | | |
| Gelation time at | 10.0 | 9.2 | 10.3 |
| ambient | | | |
| % Swelling at 1 | 36.8 | 35.8 | 32.4 |
| day | | | |

As shown in Table 5, the polymer compositions prepared in multiple tests from different batches of PEG-trilysine show consistent gelation time and swelling characteristics.

### Example 15: Reproducibility of Drug Release Data

Polymer compositions D1-D4, similar to C1-C3, were prepared, including the active agent dexamethasone (12 mg/0.2 mL). To determine drug release, an insert containing 0.2 mL of the sample was placed in 200 mL PBS (“receptor fluid”) and incubated at 37° C. All samples were run using n=6. At Day 1 and then at weekly time points, receptor fluid was collected for active agent concentration measurement by reverse phase high performance liquid chromatography (RP-HPLC). FIG. 9 demonstrates that the drug release profile is consistent, using four separate experiments using different batches of PEG-trilysine.

### Example 16: pH, Osmolality, and Resorption

The gelation and chemical characteristics of polymer compositions D1-D8 were determined. Gelation time, injectability, and swelling were determined as described in Example 14. Resorption was measured gravimetrically, and resorption was determined when the sample weight in the insert is less than the initial sample weight. The pH of the gel was measured indirectly by equilibrating 0.2 mL of the sample with 1 mL purified distilled water overnight at room temperature, then measuring the pH of the water. The osmolality of the sample was measured using vapor pressure osmometry.

The results of these measurements are shown in Table 6.

TABLE 6

| | D1 | D2 | D3 | D4 | D5 | D6 | D7 | D8 |
|---|---|---|---|---|---|---|---|---|
| PEG-trilysine batch ID | C | D | E | F | E | E | E | E |
| Gelation time | 14.1 | 11.7 | 11.2 | 10.2 | 11.0 | 13.7 | 16.7 | 17.0 |
| (minutes) at ambient | | | | | | | | |
| Injectable after 10 | Yes | Yes | Yes | Yes | Yes | Yes | Yes | Yes |
| min at ambient | | | | | | | | |
| Gelation time | 3 | 2 | 1 | 1 | 2 | 3 | 4 | 4 |
| (minutes) at 37° C. | | | | | | | | |
| Swelling (%) at Day 1 | 35.3 | 34.6 | 39.9 | 34.5 | 32.4 | 34.4 | 39.1 | 39.1 |
| Swelling (%) at Day 7 | 58.3 | 52.1 | 68.5 | 52.8 | 64.5 | 65.0 | 73.5 | 67.9 |
| pH of water | 6.28 | 6.39 | 6.38 | 6.32 | ND | ND | ND | ND |
| equilibrated with gel | | | | | | | | |
| Osmolality of gel | 588 | 579 | 586 | 582 | ND | ND | ND | ND |
| (mOsmol/kg) | | | | | | | | |
| Resorption time | 8 | 8 | 6 | 8 | 6 | 6 | 6 | 6 |
| (days) at 50° C. | | | | | | | | |

ND = Not Determined

### Example 17. Exemplary Formulations

Exemplary formulations for extended release otic compositions are shown below in Table 7. A formulation with Exemplary Value C for all components was generated.

TABLE 7

| | | Exemplary | Exemplary | Exemplary |
|---|---|---|---|---|
| | | Range A | Range B | Value C |
| | Function | (% w/w) | (% w/w) | (% w/w) |
| Water for | Diluent | 29.5-94.9 | 76.6-91.8 | 82.3 |
| injection | | | | |
| NHS-PEG | Polymer gel | 5-15 | 7-9 | 8.3 |
| | precursor | | | |
| Active agent (e.g., | Active | 0.01-40 | 1-10 | 6.0 |
| dexamethasone) | pharmaceutical | | | |
| | ingredient | | | |
| Sodium borate | Buffer Reagent | 0.01-3.0 | 0.05-2.0 | 1.2 |
| decahydrate | | | | |
| Sodium phosphate | Buffer Reagent | 0.01-3.0 | 0.05-2.0 | 1.1 |
| Phosphoric acid | Buffer Reagent | 0.01-3.0 | 0.05-2.0 | 0.9 |
| Trilysine acetate | Polymer gel | 0.05-0.6 | 0.1-0.3 | 0.2 |
| | crosslinker | | | |
| FD&C Blue #1 | Visualization | 0-0.5 | 0-0.05 | 0.01 |
| | dye | | | |
| Butylated | Preservative | 0-0.01 | 0-0.005 | 0.002 |
| hydroxytoluene | | | | |
| (BHT) | | | | |

### Example 18: 8-Week Safety and Pharmacokinetic Study of Intratympanic PEG-Trilysine Polymer in Hartley Guinea Pigs

An 8-week toxicology study was performed to assess the safety and pharmacokinetics of intratympanic administration of a PEG-trilysine polymer formulation in Hartley guinea pigs. Formulations with 6 wt/% dexamethasone were prepared as described in Example 8. In this study, plasma and perilymph drug levels along with visual observations of formulation localization to the RW membrane were collected and analyzed to confirm exposure. Hartley guinea pigs of both sexes [n=46 (23 males and 23 females) were used for the study. Following 3 to 7 days of acclimation, all guinea pigs underwent baseline ABRs tests at 4, 10, and 20 kHz. Three days after baseline ABR testing, the guinea pigs were divided into 4 treatment groups as follows: 1) PEG-trilysine polymer formulation including dexamethasone, 2) PEG-trilysine polymer formulation-vehicle, 3) saline, and 4) gentamicin IT injection. Gentamicin is included as a positive control with a known ototoxicity profile.

Table 8 summarizes the study design. Groups 1-3 were further divided into subgroups with varying survival times up to 8 weeks. The number of ears for the 4-week and 8-week timepoints in this study were powered to detect a 20 dB shift from baseline in the ABR threshold measurement at each of the three test frequencies (1-way t-test; independent means; a priori; power=0.95), using sample data to estimate variability from pilot studies. Auditory brainstem responses and blood sampling were collected at specified in-life timepoints, including terminus. Perilymph sampling, middle ear visualization, and histology sample preparation were performed on specified animals at terminus.

TABLE 8
Summary of Study Design for 8-Week Safety and Pharmacokinetic
Study of PEG-trilysine polymer in Guinea Pigs

| Treatment | Injection | | | Study Procedures | | | | | | |
|---|---|---|---|---|---|---|---|---|---|---|
| Group | Volume | | N (ears) | Baseline | Day 0 | Day 3 | Week 1 | Week 2 | Week 4 | Week 8 |
| | 0.005 | mL | 4 | a | x | a, c, e | | | | |
| | | | 4 | a | x | — | a, c, e | | | |
| | | | 4 | a | x | — | — | — | a, d | |
| | | | 4 | a | x | — | — | — | a, c, e | |
| | | | 10 | a | x | a, b | a, b | — | a, b | a, b, d |
| | | | 10 | a | x | a, b | a, b | — | a, b | a, b, c, e |
| | 0.005 | mL | 4 | a | x | a, b | a | — | a, d | |
| | | | 4 | a | x | a, b | a | — | a, e | |
| | | | 6 | a | x | a | a | — | a | a, d |
| | | | 6 | a | x | a | a | — | a | a, e |
| | 0.005 | mL | 4 | a | x | a | a | — | a, d | |
| | | | 4 | a | x | a | a | — | a, e | |
| | | | 6 | a | x | a | a | — | a | a, d |
| | | | 6 | a | x | a | a | — | a | a, e |
| | 0.050 | Ml | 4 | a | x | a | — | a, d | | |

Study Procedures:
a = Auditory Brainstem Response (ABR)
b = Plasma Collection for Drug Content
c = Perilymph Collection for Drug Content
d = Cochlear Histology for Hair Cell Counts
e = Middle Ear Histology Prep
x = Intratympanic Injection

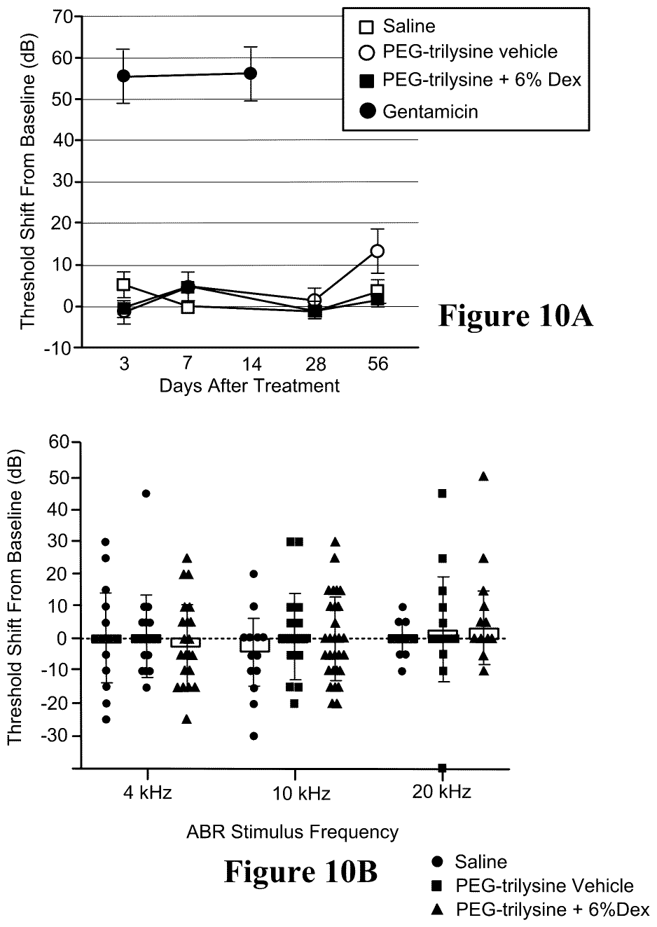

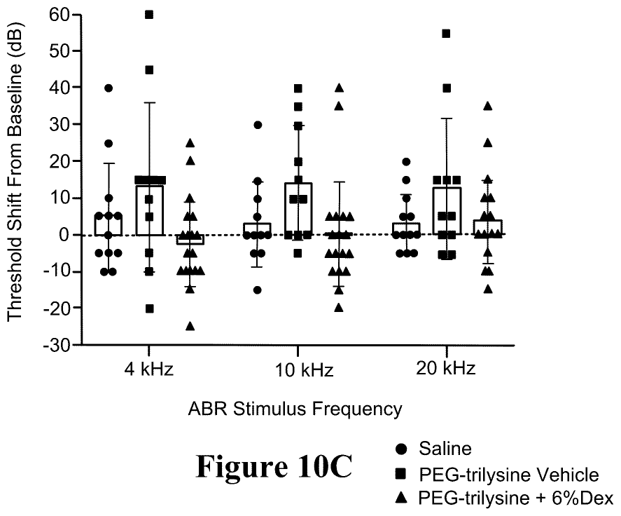

Changes in hearing as assessed by ABR were minimal following IT injection of 5 μL of PEG-trilysine polymer vehicle with and without 6% dexamethasone at all timepoints tested (FIG. 10A) and were not statistically different than the saline control groups. The positive control gentamicin treatment produced a robust 55+ dB threshold shift at Days 3 and 14. The small threshold elevations seen across the other treatment groups, are consistent with sequelae of a rodent intratympanic injection procedure, which can produce transient mild elevation in ABR thresholds resulting from a recent TM perforation.

The two timepoints that were highest powered were the 4-week and 8-week post injections measurements. At the 4-week timepoint (FIG. 10B), statistical analysis showed no significant main effects of Treatment Group or ABR Test Frequency, and no interaction between Treatment Group and Frequency. None of the follow-up multiple comparisons were significant. Similarly, at the 8-week timepoint (FIG. 10C), there was no main effect for ABR Test Frequency, F (1.85, 76.0)=0.365, p=0.679, nor was there a Treatment Group×Test Frequency interaction, F (4, 82)=1.18, p=0.325. There was a significant main effect for Treatment Group, F (2, 41)=3.65, p=0.035. These results suggest that ABR Threshold shifts at the 8-week time point were significantly different between treatment groups, regardless of the ABR test frequency. However, follow-up Tukey's multiple comparisons showed that there were no significant differences between any of the groups. The trends closest to reaching statistical significance were found between PEG-trilysine polymer formulation vehicle vs PEG-trilysine polymer formulation (including 6% Dex) at the 10 kHz test frequency (p=0.062), at the 4 kHz test frequency (p=0.126), and between saline and PEG-trilysine polymer formulation vehicle at the 10 kHz test frequency (p=0.151).

Outer ear otoscopy revealed what would normally be expected after TM injections; slight TM erythema and edema 3 days after injection which progressively cleared up at longer time points until it was essentially gone in nearly all ears by 56 days. Visual observation of the middle ear space at terminus revealed that PEG-trilysine polymer formulation gel presence in the injection location was consistently observed 3-7 days after injection but not at the final 8-week timepoint.

Dexamethasone levels in guinea pig plasma and perilymph samples are summarized in Table 9. Longitudinal dexamethasone concentrations in plasma were collected in 10 animals over 8 weeks. Perilymph samples were collected at terminus at 3 days, 1 week, 4 weeks, and 8 weeks post administration. Dexamethasone levels were assayed by high performance liquid chromatography mass spectrometry (HPLC-MS), with limits of quantitation for plasma and perilymph of approximately 1 ng/mL and 7 ng/mL, respectively.

TABLE 9
Summary of Dexamethasone Levels in Plasma and Perilymph

| | Perilymph | | | Plasma | | |
|---|---|---|---|---|---|---|
| | Mean | | | Mean | | |
| | (ng/mL) | Std Error | n | (ng/mL) | Std Error | n |
| 3 days | 109 | 60 | 5* | 5.5 | 1.1 | 10 |
| 1 week | 1698 | 1086 | 4 | 3.2 | 0.3 | 10 |
| 4 weeks | BLQ | — | 4 | BLQ | — | 10 |
| 8 weeks | BLQ | — | 10 | BLQ | — | 10 |

BLQ = below limit of quantitation
*The injection in one ear in the 3 day group was not successful and an additional animal brought on study. The contralateral ear was carried forward, resulting in n = 5 rather than the planned n = 4 in this group.

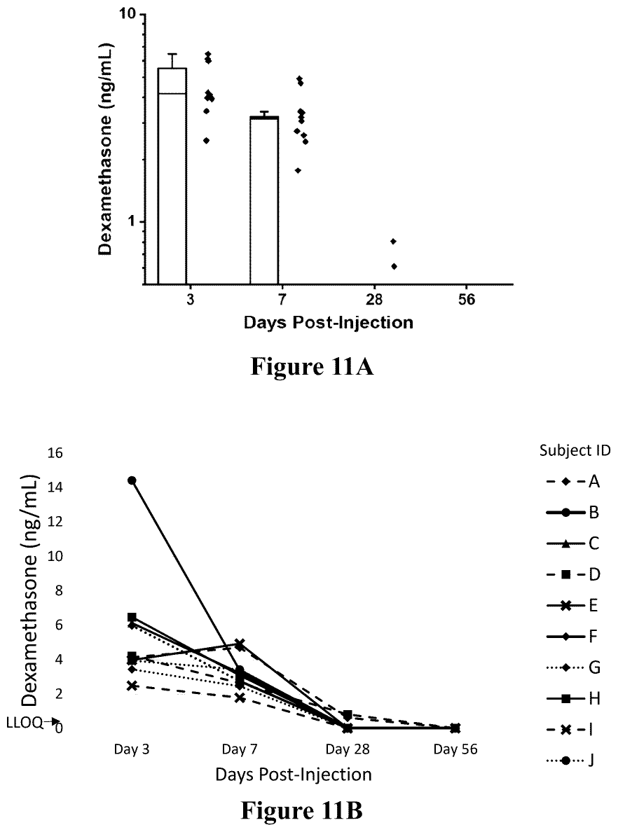

Plasma dexamethasone concentrations were consistently highest at 3 days post-injection and decreased over time, dropping below the limit of quantitation (BLQ) by 8 weeks in all animals (FIGS. 11A and 11B).

Perilymph samples at 3 days after treatment revealed considerably higher levels than in plasma: 20-fold and then 500-fold higher mean levels at 3 days and 7 days, respectively, consistent with previous results in Example 10. Perilymph concentrations were highly variable in this study, due at least in part to low sample volumes (as low as 1.5 microliters which accounts for less than 40% of the scala tympani volume) and post mortem sample collection. Post mortem collection was necessary to minimize impact on safety interpretation but introduces risks of variable sample dilution arising from entry of CSF into the scala tympani during tissue dissection. In light of these factors, middle ear observations were recorded during dissection of the tympanic bulla to confirm accurate placement of the product. PEG-trilysine polymer formulation and PEG-trilysine polymer formulation vehicle samples were readily observed in the round window niche in all specimens at the 3 and 7 day timepoints. These observations, taken together with the perilymph dexamethasone levels, support the intended exposure of the cochlea to the administered drug product.

1. A method of treating an otic disease or disorder in a subject, the method comprising:
identifying a subject as having an otic disease or disorder; and
administering through an otic delivery cannula a dosage of about 40 μL to about 60 μL of an extended release otic dexamethasone composition into contact with a round window membrane of a cochlea of the subject while the extended release otic dexamethasone composition is a liquid substance, said extended release otic dexamethasone composition transitioning to a gel substance after said contact with the round window membrane of the cochlea of the subject to thereby remain at the round window membrane of the cochlea for sustained release of dexamethasone into the cochlea of the subject, wherein said extended release otic dexamethasone composition comprises:
said dexamethasone in an amount of about 1% to about 15% by weight of the extended release otic composition;
and a cross-linking polymer composition having a pH of about 5.5 to about 6.0 and a gelation time of about 2 minutes to about 8 minutes at a temperature of about 37° C., said cross-linking polymer composition comprising: (i) a first functional polymer in an amount of about 5% to about 15% by weight of the polymer composition, (ii) trilysine, or a salt thereof, in an amount of about 0.05% to about 0.6% by weight of the polymer composition so as to provide a gel formation reaction with said first functional polymer, and (iii) water;
wherein the extended release otic dexamethasone composition has a residence time of at least 5 days as the gel substance at the round window membrane of the cochlea of the subject.

2. The method of claim 1, wherein said administering comprises urging the extended release otic dexamethasone composition from a syringe and through the otic delivery cannula having a diameter of 23 G or smaller while the extended release otic dexamethasone composition is the liquid substance.

3. The method of claim 2, further comprising, prior to said administering, inserting the otic delivery cannula having the diameter of 23 G or smaller through a tympanic membrane and toward the round window membrane of the cochlea of the subject.

4. The method of claim 2, wherein said administering comprises inserting the otic delivery cannula toward the round window membrane of the cochlea of the subject through a mastoidectomy approach.

5. The method of claim 1, wherein said administering comprises depositing the dosage of about 40 μL to about 60 μL of the extended release otic dexamethasone composition into contact with the round window membrane of the cochlea of the subject while the extended release otic dexamethasone composition does not contact any of ossicles of the subject.

6. The method of claim 1, further comprising, during said administering, providing direct visualization of the dosage of the extended release otic dexamethasone composition being deposited into contact with the round window membrane of the cochlea of the subject.

7. The method of claim 6, providing direct visualization of the dosage of the extended release otic dexamethasone composition comprises using an endoscope instrument.

8. The method of claim 1, wherein said extended release otic dexamethasone composition transitioning remains at the round window membrane of the cochlea for sustained release of dexamethasone into the cochlea of the subject for a period of about 5 days to about 2 months.

9. The method of claim 1, further comprising, prior to said administering the dosage of about 40 μL to about 60 μL of the extended release otic dexamethasone composition, using delayed intravenous gadolinium contrast enhanced 3T MRI to evaluate the subject for a first level of endolymphatic hydrops, perilymphatic enhancement, or both.

10. The method of claim 9, further comprising, after said administering the dosage of about 40 μL to about 60 μL of the extended release otic dexamethasone composition, using a second instance of delayed intravenous gadolinium contrast enhanced 3T MRI to evaluate the subject for a subsequent level of endolymphatic hydrops, perilymphatic enhancement, or both.

11. The method of claim 10, wherein said second instance of delayed intravenous gadolinium contrast enhanced 3T MRI provides an evaluation of the subject for the subsequent level about fifteen days after said administering the dosage.

12. The method of claim 1, wherein said first functional polymer comprises pentaerythritol poly(ethylene glycol) ether tetrasuccinimidyl glutarate in an amount of about 5% to about 15% by weight of the polymer composition.

13. The method of claim 1, wherein the extended release otic dexamethasone composition transitions to the gel substance that is elastic or mucoadhesive.

14. The method of claim 1, wherein the extended release otic dexamethasone composition transitions to the gel substance that has an osmolality of about 300 mOsmol/kg to about 600 mOsmol/kg.

15. The method of claim 1, wherein the gel substance is configured to swell less than 100% following equilibration in phosphate-buffered saline (PBS) for 1 day.

16. The method of claim 1, wherein said dexamethasone is present in an amount of about 1% to about 10% by weight of said extended release otic dexamethasone composition.

17. The method of claim 16, wherein said trilysine, or the salt thereof, is present in an amount of about 0.1% to about 0.3% by weight of the polymer composition so as to provide the gel formation reaction with said first functional polymer.

18. The method of claim 1, wherein said extended release otic dexamethasone composition further comprises: a dye mixed with said dexamethasone, said first functional polymer, said trilysine, or the salt thereof, and said water.

19. The method of claim 1, wherein said identifying comprises identifying that the subject has the otic disease or disorder is selected from the group consisting of Ménière's Disease (MD), Autoimmune Inner Ear Disease (AIED), sudden sensorineural hearing loss (SSNHL), noise-induced hearing loss (NIHL), age-related hearing loss, sensorineural hearing loss associated with diabetes, tinnitus, damaged cilia from an autoimmune disorder, damaged cilia from an infection, damaged cilia from excess fluid or pressure, hearing loss due to chemotherapy, and combinations thereof.


SEQUENCE LISTING (8 sequences)

SEQ ID NO: 1 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 3 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 4 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 5 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 6 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 7 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 8 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR